(19) United States
(12) Patent Application Publication
(10) Pub. No.: US 2026/0151474 A1
(43) Pub. Date: Jun. 4, 2026

(54) CH505 ENVELOPES TO ENGAGE AND MATURE CD4 BINDING SITE NEUTRALIZING ANTIBODIES

(71) Applicant: Duke University, Durham, NC (US)

(72) Inventors: Barton F. Haynes, Durham, NC (US); Kevin O. Saunders, Durham, NC (US); David Montefiori, Durham, NC (US); Kevin J. Wiehe, Durham, NC (US)

(21) Appl. No.: 18/870,626

(22) PCT Filed: Jun. 1, 2023
(86) PCT No.: PCT/US2023/067797

§ 371 Date: Nov. 29, 2024

Related U.S. Application Data
(60) Provisional application No. 63/347,833, filed on Jun. 1, 2022.

(51) Int. Cl. C07K 16/00; C12N 7/00

(52) U.S. Cl. CPC A61K 39/21; A61P 37/04; C07K 14/005; A61K 2039/53; A61K 2039/55572; A61K 2039/70; C12N 2740/16122; C12N 2740/16134; C12N 2740/16171

(57) ABSTRACT

In certain aspects the invention provides HIV-1 immunogens, including HIV-1 envelopes with optimized sequences for antibody induction.

This International Patent Application claims the benefit of and priority to U.S. Application No. 63/347,833, filed Jun. 1, 2022, entitled “CH505 envelopes to engage and mature CD4 binding site neutralizing antibodies,” the content of which is hereby incorporated by reference in its entirety.

This invention was made with government support under Center for HIV/AIDS Vaccine Immunology-Immunogen Design grant UM1 AI144371 from the NIH, NIAID, Division of AIDS. The government has certain rights in the invention.

### TECHNICAL FIELD

The present invention relates in general, to a composition suitable for use in inducing anti-HIV-1 antibodies, and, in particular, to immunogenic compositions comprising envelope proteins and nucleic acids to induce cross-reactive neutralizing antibodies and increase their breadth of coverage. The invention also relates to methods of inducing such broadly neutralizing anti-HIV-1 antibodies using such compositions.

### BACKGROUND

The development of a safe and effective HIV-1 vaccine is one of the highest priorities of the scientific community working on the HIV-1 epidemic. While anti-retroviral treatment (ART) has dramatically prolonged the lives of HIV-1 infected patients, ART is not routinely available in developing countries.

### SUMMARY OF THE INVENTION

In certain embodiments, the invention provides compositions and methods for induction of immune response, for example cross-reactive (broadly) neutralizing Ab induction.

In certain aspects, the invention provides CH505 envelope immunogens comprising mutations permitting engagement of CH235 unmutated common ancestors (UCAs) and/or initiation of CD4 binding site CH235-like antibodies. In non-limiting embodiments, the CH505 envelope is CH505 M5 G458Y N197D envelope. Throughout this patent application, CH505 TF N279K, which is CH505 TF envelope sequence comprising N279K substitution, is interchangeably referred also as CH505 M5 and/or CH505 TF M5(N279K).

In certain embodiments, the compositions contemplate nucleic acid, as DNA and/or RNA, or proteins immunogens either alone or in any combination. In certain embodiments, the methods contemplate genetic, as DNA and/or RNA, immunization either alone or in combination with envelope protein(s).

In certain embodiments, the compositions include one or more protein or polypeptide disclosed in FIGS. 21-24, Tables 1 and 3. In certain embodiments, the compositions include one or more DNA disclosed in FIGS. 21-24, Tables 1 and 3. In certain embodiments, the compositions include one or more RNA or mRNA disclosed in FIG. 25, Table 4. In certain embodiments, the compositions include any combination of the one or more protein or polypeptide of FIGS. 21-24, Tables 1 and 3 and/or one or more DNA disclosed in FIGS. 21-24, Tables 1 and 3 and/or one or more RNA or mRNA disclosed in FIG. 25, Table 4. In certain embodiments, the compositions include any combination of the one or more coding regions of RNA or mRNA disclosed in FIG. 25, Table 4. In certain embodiments, the compositions include any of the one or more coding regions of RNA or mRNA disclosed in FIG. 25, Table 4 located between a 5′UTR sequence and 3′UTR sequence provided in Table 5. The untranslated regions (UTRs) can be substituted with alternative UTRs recognized in the art.

In certain embodiments, the nucleic acid encoding an envelope is operably linked to a promoter inserted an expression vector. In certain aspects the compositions comprise a suitable carrier. In certain aspects the compositions comprise a suitable adjuvant.

In certain embodiments, the induced immune response includes induction of antibodies, including but not limited to autologous and/or cross-reactive (broadly) neutralizing antibodies against HIV-1 envelope. Various assays that analyze whether an immunogenic composition induces an immune response, and the type of antibodies induced are known in the art and are also described herein.

In certain aspects the invention provides a nucleic acid sequence encoding any of the polypeptides of the invention, wherein the nucleic acid is operably linked to a promoter. In certain aspects the invention provides a nucleic acid consisting essentially of a nucleic acid sequence encoding any of the polypeptides of the invention, wherein the nucleic acid is operably linked to a promoter. In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain aspects the invention provides an expression vector consisting essentially a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In certain aspects the invention provides a composition comprising at least one nucleic acid encoding HIV-1 envelope of the invention. In certain embodiments the at least one nucleic acid is an RNA or mRNA. In certain embodiments at least one RNA or mRNA is at least one RNA or mRNA disclosed in FIG. 25, Table 4. In certain embodiments at least one RNA or mRNA is at least one coding regions of RNA or mRNA disclosed in FIG. 25, Table 4. In certain embodiments, the compositions include any of the one or more coding regions of RNA or mRNA disclosed in FIG. 25, Table 4 located between a 5′UTR sequence and 3′UTR sequence provided in Table 5. The untranslated regions (UTRs) can be substituted with alternative UTRs recognized in the art.

In certain embodiments, the compositions and methods employ an HIV-1 envelope as polypeptide instead of a nucleic acid sequence encoding the HIV-1 envelope. In certain embodiments, the compositions and methods employ an HIV-1 envelope as polypeptide, a nucleic acid sequence encoding the HIV-1 envelope, or a combination thereof. In certain embodiments, the polypeptides are recombinantly produced.

The envelope used in the compositions and methods of the invention can be a gp160, gp150, gp145, gp140, gp120, gp41, N-terminal deletion variants as described herein, cleavage resistant variants as described herein, or codon optimized sequences thereof. In certain embodiments the composition comprises envelopes as trimers. In certain embodiments, an envelope is provided as a fusion protein that can self-assemble into a multimeric complex. In certain embodiments, envelope proteins are multimerized, for example trimers are attached to a particle such that multiple copies of the trimer are attached and the multimerized envelope is prepared and formulated for immunization in a human. In certain embodiments, the compositions comprise envelopes, including but not limited to trimers as particulate, high-density array on liposomes or other particles, for example but not limited to nanoparticles. In some embodiments, the trimers are in a well ordered, near native like or closed conformation. In some embodiments the trimer compositions comprise a homogenous mix of native like trimers. In some embodiments the trimer compositions comprise at least 65%, 70%, 75%, 80%, 85%, 90%, 95% native like trimers.

The polypeptide contemplated by the invention can be a polypeptide comprising any one of the polypeptides described herein. The polypeptide contemplated by the invention can be a polypeptide consisting essentially of any one of the polypeptides described herein. The polypeptide contemplated by the invention can be a polypeptide consisting of any one of the polypeptides described herein. In certain embodiments, the polypeptide is recombinantly produced. In certain embodiments, the invention provides nucleic acid sequences encoding any of the polypeptides of the invention. In certain embodiments, the nucleic acids are mRNAs, including without limitation any suitable modified mRNA. In certain embodiments, the nucleic acids are self-replicating nucleic acids. In certain embodiments, the polypeptides and nucleic acids of the invention are suitable for use as an immunogen, for example to be administered in a human subject.

In certain embodiments the envelope is any of the forms of HIV-1 envelope. In certain embodiments the envelope is gp120, gp140, gp145 (i.e. with a transmembrane domain), gp150, gp160. In certain embodiments, the gp140 form is designed to form a stable trimer. In certain embodiments envelope protomers from a trimer which is not a SOSIP trimer. In certain embodiment the trimer is a SOSIP based trimer wherein each protomer comprises additional modifications. In certain embodiments, envelope trimers are recombinantly produced. In certain embodiments, envelope trimers are purified from cellular recombinant fractions by antibody binding and reconstituted in lipid comprising formulations. See for example WO2015/127108 titled “Trimeric HIV-1 envelopes and uses thereof” which content is herein incorporated by reference in its entirety. In certain embodiments the envelopes of the invention are engineered and comprise non-naturally occurring modifications.

In certain embodiments, the envelope is in a liposome. In certain embodiments the envelope comprises a transmembrane domain with a cytoplasmic tail embedded in a liposome. In certain embodiments, the nucleic acid comprises a nucleic acid sequence which encodes a gp120, gp140, gp145, gp150, gp160.

In certain embodiments, where the nucleic acids are operably linked to a promoter and inserted in a vector, the vectors are any suitable vector. Non-limiting examples include, VSV, replicating rAdenovirus type 4, MVA, Chimp adenovirus vectors, pox vectors, and the like. In certain embodiments, the nucleic acids are administered in NanoTaxi block polymer nanospheres. In certain embodiments, the composition and methods comprise an adjuvant. Non-limiting examples include, AS01 B, AS01 E, gla/SE, alum, Poly I poly C (poly IC), polyIC/long chain (LC) TLR agonists, TLR7/8 and 9 agonists, or a combination of TLR7/8 and TLR9 agonists (see Moody et al. (2014) J. Virol. March 2014 vol. 88 no. 6 3329-3339), or any other adjuvant. Non-limiting examples of TLR7/8 agonist include TLR7/8 ligands, Gardiquimod, Imiquimod and R848 (resiquimod). A non-limiting embodiment of a combination of TLR7/8 and TLR9 agonist comprises R848 and oCpG in STS (see Moody et al. (2014) J. Virol. March 2014 vol. 88 no. 6 3329-3339). In particular non-limiting embodiments, the TLR-7,8 adjuvant 3M052 can be used (See e.g. 3M-052, a synthetic TLR-7/8 agonist, induces durable HIV-1 envelope-specific plasma cells and humoral immunity in nonhuman primates. Kasturi S P, Rasheed M A U, Havenar-Daughton C, Pham M, Legere T, Sher Z J, Kovalenkov Y, Gumber S, Huang J Y, Gottardo R, Fulp W, Sato A, Sawant S, Stanfield-Oakley S, Yates N, LaBranche C, Alam S M, Tomaras G, Ferrari G, Montefiori D, Wrammert J, Villinger F, Tomai M, Vasilakos J, Fox C B, Reed S G, Haynes B F, Crotty S, Ahmed R, Pulendran B. Sci Immunol. 2020 Jun. 19; 5(48):eabb1025. doi: 10.1126/sciimmunol.abb1025.PMID: 32561559; Breadth of SARS-CoV-2 Neutralization and Protection Induced by a Nanoparticle Vaccine. Li D, Martinez D R, Schafer A, Chen H, Barr M, Sutherland L L, Lee E, Parks R, Mielke D, Edwards W, Newman A, Bock K W, Minai M, Nagata B M, Gagne M, Douek D, DeMarco C T, Denny T N, Oguin T H, Brown A, Rountree W, Wang Y, Mansouri K, Edwards R J, Ferrari G, Sempowski G D, Eaton A, Tang J, Cain D W, Santra S, Pardi N, Weissman D, Tomai M, Fox C, Moore I N, Andersen H, Lewis M G, Golding H, Khurana S, Seder R, Baric R S, Montefiori D C, Saunders K O, Haynes BF.bioRxiv. 2022 Feb. 14:2022.01.26.477915. doi: 10.1101/2022.01.26.477915. Preprint.PMID: 35118474).

In non-limiting embodiments, the adjuvant is an LNP. See e.g., without limitation Shirai et al. “Lipid Nanoparticle Acts as a Potential Adjuvant for Influenza Split Vaccine without Inducing Inflammatory Responses” Vaccines 2020, 8, 433; doi:10.3390/vaccines8030433, published 3 Aug. 2020. In non-limiting embodiments, LNPs used as adjuvants for proteins or mRNA compositions are composed of an ionizable lipid, cholesterol, lipid conjugated with polyethylene glycol, and a helper lipid. Non-limiting embodiment include LNPs without polyethylene glycol.

In certain aspects the invention provides a cell comprising a nucleic acid encoding any one of the envelopes of the invention suitable for recombinant expression. In certain aspects, the invention provides a clonally derived population of cells encoding any one of the envelopes of the invention suitable for recombinant expression. In certain aspects, the invention provides a stable pool of cells encoding any one of the envelopes of the invention suitable for recombinant expression.

In certain aspects, the invention provides a HIV-1 envelope polypeptide listed in Table 1 or Table 3. In certain embodiments, the polypeptide is a non-naturally occurring protomer designed to form an envelope trimer. In certain embodiments, the envelopes are gp160 transmembrane proteins. The invention also provides nucleic acids encoding these polypeptides. Non-limiting examples of amino acids and nucleic acids of such protomers are shown in FIGS. 21-24.

In certain aspects the invention provides a recombinant trimer comprising three identical protomers of an envelope from Table 1 or Table 3. In certain aspects the invention provides an immunogenic composition comprising the recombinant trimer and a carrier, wherein the trimer comprises three identical protomers of an HIV-1 envelope listed in Table 1 or Table 3. In certain aspects the invention provides an immunogenic composition comprising nucleic acid encoding these HIV-1 envelopes and a carrier.

In certain aspects the invention provides nucleic acids encoding HIV-1 envelopes for immunization wherein the nucleic acid encodes a gp120 envelope, gp120D8 envelope, a gp140 envelope (gp140C, gp140CF, gp140CFI) as soluble or stabilized protomer of a SOSIP trimer, a gp145 envelope, a gp150 envelope, or a transmembrane bound envelope, e.g. gp160 envelope.

In certain aspects the invention provides a selection of HIV-1 envelopes for immunization wherein the HIV-1 envelope is a gp120 envelope or a gp120D8 variant. In certain embodiments a composition for immunization comprises protomers that form stabilized SOSIP trimers.

In certain embodiments, the compositions for use in immunization further comprise an adjuvant.

In certain embodiments, wherein the compositions comprise a nucleic acid, the nucleic acid is operably linked to a promoter, and could be inserted in an expression vector. In certain embodiments, the nucleic acid is a mRNA. In certain embodiments, the nucleic acid is encapsulated in a lipid nanoparticle.

In one aspect the invention provides a composition for a prime boost immunization regimen comprising one or more envelopes from Table 1 or Table 3, wherein in some embodiments the polypeptide is a non-naturally occurring protomer designed to form an envelope trimer, wherein the envelope is a prime or boost immunogen, and wherein in some embodiments the polypeptide is non-naturally occurring gp160 envelope, e.g. a transmembrane envelope. In one aspect the invention provides a composition for a prime boost immunization regimen comprising one or more envelopes of the invention.

In certain aspects the invention provides methods of inducing an immune response in a subject comprising administering a composition comprising a polypeptide and/or any suitable form of a nucleic acid(s) encoding an HIV-1 envelope(s) in an amount sufficient to induce an immune response.

In certain embodiments the method further comprises administering a composition comprising one or more envelopes from Table 3 as a boost, wherein the envelope is administered as a polypeptide or a nucleic acid encoding the same. In some embodiments, the nucleic acid encoding the one or more envelopes from Table 3 is an mRNA from FIG. 25, Table 4. In certain embodiments, the nucleic acid encoding the one or more envelopes from Table 3 is the coding regions of mRNA from FIG. 25, Table 4. In certain embodiments, the compositions include any of the one or more coding regions of RNA or mRNA disclosed in FIG. 25, Table 4 located between a 5′UTR sequence and 3′UTR sequence provided in Table 5. The untranslated regions (UTRs) can be substituted with alternative UTRs recognized in the art. In some embodiments, the method further comprises administering a composition comprising any one of HIV envelope referred to as w24 or any combination thereof as a boost, wherein the envelope is administered as a polypeptide or a nucleic acid encoding the same. In some embodiments the method further comprises administering a composition comprising envelope CH505.w24.e5 F14.SOS.GSA.L.Y712I.I535M.A316W.S306L.R308L gp160_mVHss as a boost, wherein the envelope is administered as a polypeptide or a nucleic acid encoding the same. In some embodiments the method further comprises administering a composition comprising envelope CH505.TF_N197D_F14(A204V_V208L_V68I_V255L)_SOS.GS.L_Y712I_mVHss gp160 as a boost, wherein the envelope is administered as a polypeptide or a nucleic acid encoding the same.

In certain embodiments, the nucleic acid encodes a gp120 envelope, gp120D8 envelope, a gp140 envelope (gp140C, gp140CF, gp140CFI) as soluble or stabilized protomer of a SOSIP trimer, a gp145 envelope, a gp150 envelope, or a transmembrane bound envelope. In certain embodiments, the polypeptide is gp120 envelope, gp120D8 envelope, a gp140 envelope (gp140C, gp140CF, gp140CFI) as soluble or stabilized protomer of a SOSIP trimer, a gp145 envelope, a gp150 envelope, or a gp160 envelope including without limitation transmembrane bound envelope.

In certain embodiments, the methods comprise administering an adjuvant. In certain embodiments, the methods comprise administering an agent which modulates host immune tolerance. In certain embodiments, the administered polypeptide is multimerized in a liposome or nanoparticle. In certain embodiments, the methods comprise administering one or more additional HIV-1 immunogens to induce a T cell response. Non-limiting examples include gag, nef, pol, etc.

In certain aspects, the invention provides a recombinant HIV-1 Env ectodomain trimer, comprising three gp120-gp4l protomers comprising a gp120 polypeptide and a gp41 ectodomain, wherein each protomer is the same and each protomer comprises portions from envelope BG505 HIV-1 strain and gp120 polypeptide portions from a CH505 HIV-1 strain and stabilizing mutations A316W and E64K. Non-limited examples of envelopes contemplated as trimers are listed in Table 1 or Table 3. In some embodiments, the amino acid sequence of one monomer comprised in the trimer is shown in FIGS. 21-24. In some embodiments, the trimer is immunogenic. In some embodiments the trimer binds to any one of the antibodies PGT145, PGT151, CH235UCA, CH235.12, VRC01, PGT128, or any combination thereof. In some embodiments the trimer does not bind to antibody 19B and/or 17B.

In certain aspects, the invention provides a pharmaceutical composition comprising any one of the recombinant trimers of the invention. In certain embodiments the compositions comprising trimers are immunogenic. The percent trimer in such immunogenic compositions could vary. In some embodiments the composition comprises 70%, 71%, 72%, 73%, 74%, 75%, 76%, 77%, 78%, 79%, 80%, 81%, 82%, 83%, 84%, 85%, 86%, 87%, 89%, 90%, 91%, 92%, 93%, 94%, 95%, 96%, 97%, 98%, or 99% stabilized trimer.

In certain embodiments, the envelope comprise ferritin. In certain embodiments, the inventive designs comprise modifications, including without limitation linkers between the envelope and ferritin designed to optimize ferritin nanoparticle assembly.

In certain aspects, the invention provides a composition comprising any one of the inventive envelopes or nucleic acid sequences encoding the same. In certain embodiments, the nucleic acid is mRNA. In certain embodiments, the mRNA is comprised in a lipid nano-particle (LNP). In some embodiments the mRNA is from FIG. 25, Table 4. In certain embodiments, the mRNAs comprise the coding regions of mRNAs disclosed in FIG. 25, Table 4. In certain embodiments, the compositions include any of the one or more coding regions of RNA or mRNA disclosed in FIG. 25, Table 4 located between a 5′UTR sequence and 3′UTR sequence provided in Table 5. The untranslated regions (UTRs) can be substituted with alternative UTRs recognized in the art.

In certain aspects, the invention provides compositions comprising a nanoparticle which comprises any one of the envelopes of the invention.

In some embodiment, the composition comprises a nanoparticle which is a ferritin self-assembling nanoparticle.

In certain aspects, the invention provides a method of inducing an immune response in a subject comprising administering an immunogenic composition comprising any one of the stabilized envelopes of the invention. In certain embodiments, the composition is administered as a prime and/or a boost. In certain embodiments, the composition comprises nanoparticles. In certain embodiments, methods of the invention further comprise administering an adjuvant.

In certain aspects, the invention provides a composition comprising a plurality of nanoparticles comprising a plurality of the envelopes/trimers of the invention. In non-limiting embodiments, the envelopes/trimers of the invention are multimeric when comprised in a nanoparticle. The nanoparticle size is suitable for delivery. In non-liming embodiments the nanoparticles are ferritin based nanoparticles.

In certain aspects, the invention provides nucleic acids comprising sequences encoding proteins of the invention. In certain embodiments, the nucleic acids are DNAs. In certain embodiments, the nucleic acids are mRNAs. In some embodiments the mRNAs are from FIG. 25, Table 4. In certain embodiments, the mRNAs comprise the coding regions of mRNAs disclosed in FIG. 25, Table 4. In certain embodiments, the compositions include any of the one or more coding regions of RNA or mRNA disclosed in FIG. 25, Table 4 located between a 5′UTR sequence and 3′UTR sequence provided in Table 5. The untranslated regions (UTRs) can be substituted with alternative UTRs recognized in the art. In certain aspects, the invention provides expression vectors comprising the nucleic acids of the invention.

In certain aspects, the invention provides a pharmaceutical composition comprising mRNAs encoding the inventive antibodies. In certain embodiments, these are optionally formulated in lipid nanoparticles (LNPs). In certain embodiments, the mRNAs are modified. Modifications include without limitations modified ribonucleotides, poly-A tail, 5′cap.

In certain aspects the invention provides nucleic acids encoding the inventive protein designs. In non-limiting embodiments, the nucleic acids are mRNA, modified or unmodified, suitable for use any use, e.g but not limited to use as pharmaceutical compositions. In certain embodiments, the nucleic acids are formulated in lipid, such as but not limited to LNPs.

In certain aspects the invention provides a composition comprising a nucleic acid encoding any of the envelope protein designs and a carrier. Also disclosed herein are modified mRNA, for example comprising suitable modifications for expression as immunogens. Non-limiting examples include modified nucleosides, capping, polyA tail, and the like. In certain embodiments, the compositions comprise an adjuvant.

In some embodiments, the invention provides a nucleic acid of FIG. 21A, 23 or 24A, or encoding a recombinant HIV-1 envelope polypeptide according to Tables 1 or 3. In non-limiting embodiments, the nucleic acid is an mRNA. In some embodiments, the mRNA comprises a nucleic acids sequence provided in FIG. 21A, 23, or 24A, wherein thymine (T) will be uridine (U). In some embodiments, the mRNA comprises a nucleic acids sequence provided in FIG. 21A, 23 or 24A, wherein thymine (T) will be 1-methyl-pseudouridine. In some embodiments, the mRNA is modified. In some embodiments, the modification is a modified nucleotide such as 5-methyl-cytidine and/or 6-methyl-adenosine and/or modified uridine. In some embodiments, the mRNA comprises a nucleic acid sequence provided in FIG. 21A, 23 or 24A and further comprises a poly A tail, wherein the poly A tail is about 85 to about 200 nucleotides long. In some embodiments, the mRNA comprises a nucleic acid sequence provided in FIG. 21A, 23 or 24A and further comprises a poly A tail, wherein the poly A tail is about 85 to about 110 nucleotides long. In some embodiments, the mRNA comprises a nucleic acid sequence provided in FIG. 21A, 23 or 24A and further comprises a poly A tail, wherein the poly A tail is about 90 to about 110 nucleotides long. In some embodiments, the mRNA comprises a nucleic acid sequence provided in FIG. 21A, 23 or 24A and further comprises a poly A tail, wherein thymine (T) will be uridine (U) and wherein the mRNA comprises a poly A tail about 85 to about 200 nucleotides long. In some embodiments, the mRNA comprises a nucleic acid sequence provided in FIG. 21A, 23 or 24A and further comprises a poly A tail, wherein thymine (T) will be uridine (U) and wherein the mRNA comprises a poly A tail about 85 to about 110 nucleotides long. In some embodiments, the mRNA comprises a nucleic acid sequence provided in FIG. 21A, 23 or 24A and further comprises a poly A tail, wherein thymine (T) will be uridine (U) and wherein the mRNA comprises a poly A tail about 90 to about 110 nucleotides long. In some embodiments, the mRNA comprises a nucleic acid sequence provided in FIG. 21A, 23 or 24A and further comprises a poly A tail, wherein thymine (T) will be 1-methyl-pseudouridine and wherein the mRNA comprises a poly A tail about 85 to about 200 nucleotides long. In some embodiments, the mRNA comprises a nucleic acid sequence provided in FIG. 21A, 23 or 24A and further comprises a poly A tail, wherein thymine (T) will be 1-methyl-pseudouridine and wherein the mRNA comprises a poly A tail about 85 to about 110 nucleotides long. In some embodiments, the mRNA comprises a nucleic acid sequence provided in FIG. 21A, 23 or 24A and further comprises a poly A tail, wherein thymine (T) will be 1-methyl-pseudouridine and wherein the mRNA comprises a poly A tail about 90 to about 110 nucleotides long. In some embodiments, the mRNA comprises a nucleic acid sequence provided in FIG. 21A, 23 or 24A located between a 5′UTR sequence and 3′UTR sequence provided in Table 5. The untranslated regions (UTRs) can be substituted with alternative UTRs recognized in the art. In non-limiting embodiments, the mRNA is administered as an LNP.

In some aspects, the invention provides a nucleic acid encoding any of the recombinant envelopes described herein. In some embodiments, the invention provides a composition comprising the nucleic acid and a carrier. In some embodiments, the nucleic acid is an mRNA. In some embodiments the mRNA is from FIG. 25, Table 4. In certain embodiments, the mRNAs comprise the coding regions of mRNAs disclosed in FIG. 25, Table 4. In certain embodiments, the compositions include any of the one or more coding regions of RNA or mRNA disclosed in FIG. 25, Table 4 located between a 5′UTR sequence and 3′UTR sequence provided in Table 5. The untranslated regions (UTRs) can be substituted with alternative UTRs recognized in the art. In some embodiments, the mRNA is encapsulated in a lipid nanoparticle (LNP).

In some embodiments, the mRNA comprises the nucleic acids according to FIGS. 25, wherein thymine (T) will be uridine (U). In some embodiments, the mRNA comprises the nucleic acids according to FIG. 25, wherein thymine (T) will be 1-methyl-pseudouridine. In some embodiments, the mRNA is modified. In some embodiments, the modification is a modified nucleotide such as 5-methyl-cytidine and/or 6-methyl-adenosine and/or modified uridine. In some embodiments, the mRNA comprises the nucleic acids according to FIG. 25 and further comprises a poly A tail, wherein the poly A tail is about 85 to about 200 nucleotides long. In some embodiments, the mRNA comprises the nucleic acids according to FIG. 25 and further comprises a poly A tail, wherein the poly A tail is about 85 to about 110 nucleotides long. In some embodiments, the mRNA comprises the nucleic acids according to FIG. 25 and further comprises a poly A tail, wherein the poly A tail is about 90 to about 110 nucleotides long. In some embodiments, the mRNA comprises the nucleic acids according to FIG. 25 and further comprises a poly A tail, wherein thymine (T) will be uridine (U) and wherein the mRNA comprises a poly A tail about 85 to about 200 nucleotides long. In some embodiments, the mRNA comprises the nucleic acids according to FIG. 25 and further comprises a poly A tail, wherein thymine (T) will be uridine (U) and wherein the mRNA comprises a poly A tail about 85 to about 110 nucleotides long. In some embodiments, the mRNA comprises the nucleic acids according to FIG. 25 and further comprises a poly A tail, wherein thymine (T) will be uridine (U) and wherein the mRNA comprises a poly A tail about 90 to about 110 nucleotides long. In some embodiments, the mRNA comprises the nucleic acids according to FIG. 25 and further comprises a poly A tail, wherein thymine (T) will be 1-methyl-pseudouridine and wherein the mRNA comprises a poly A tail about 85 to about 200 nucleotides long. In some embodiments, the mRNA comprises the nucleic acids according to FIG. 25 and further comprises a poly A tail, wherein thymine (T) will be 1-methyl-pseudouridine and wherein the mRNA comprises a poly A tail about 85 to about 110 nucleotides long. In some embodiments, the mRNA comprises the nucleic acids according to FIG. 25 and further comprises a poly A tail, wherein thymine (T) will be 1-methyl-pseudouridine and wherein the mRNA comprises a poly A tail about 90 to about 110 nucleotides long. In some embodiments, the mRNA comprises a nucleic acid sequence provided in FIG. 25 located between a 5′UTR sequence and 3′UTR sequence provided in Table 5. The untranslated regions (UTRs) can be substituted with alternative UTRs recognized in the art.

In certain embodiments, at least one of a first immunogen, a second immunogen and a third immunogen is a recombinant HIV-1 envelope polypeptide. In some embodiments, the first, second, and third immunogens are different immunogens. In certain embodiments, at least one of the first immunogen, the second immunogen and third immunogen is a recombinant trimer comprising three identical protomers of the recombinant HIV-1 envelope polypeptide. In certain embodiments, the first immunogen, the second immunogen and third immunogen are a recombinant HIV-1 envelope polypeptide. In certain embodiments, at least one of the first immunogen, the second immunogen and third immunogen is a nucleic acid. In certain embodiments, the first immunogen, the second immunogen and third immunogen are a nucleic acid. In certain embodiments, the nucleic acid is an mRNA. In certain embodiments, the mRNA is encapsulated in an LNP. In certain embodiments, the immunogenic composition further comprises one or more additional immunogens, wherein the one or more additional immunogens is different to the first, second and third immunogens.

In certain embodiments, the HIV-1 envelopes are in the form of a recombinant HIV-1 envelope polypeptides or nucleic acid, or a combination thereof. In certain embodiments, one or more of the HIV-1 envelopes is a recombinant trimer comprising three identical protomers of the recombinant HIV-1 envelope polypeptide. In certain embodiments, the nucleic acid is an mRNA. In certain embodiments, the composition comprises a carrier. In certain embodiments, the composition further comprises an adjuvant.

In certain aspects, described herein is a recombinant HIV-1 envelope polypeptide from Table 1, Table 3, FIGS. 21-24, wherein the polypeptide is a non-naturally occurring protomer designed to form an envelope trimer. In certain embodiments, the envelope is a gp160 transmembrane envelope. In some embodiments, the envelope is comprised in a multimeric complex. In certain embodiments, the envelope is based on CH505 T/F envelope and comprises an optimized sequence for binding to CH235 UCA. In certain embodiments, the envelope comprises mutations G458Y and N197X, wherein X is any amino acid which does not allow glycosylation. In certain embodiments, the envelope comprises mutations G458Y and N197D. In certain embodiments, the envelope further comprises F14 mutations A204V, V208L, V68I, and V255L. In certain embodiments, the envelope further comprises any changes at positions of interprotomer contacts within a single trimeric envelope. In certain embodiments, the envelope comprises I535M.

In certain embodiments, the envelope is linked via a linker to a self-assembling protein to form a fusion protein. In certain embodiments, the fusion protein can self assemble into a multimeric complex. In certain embodiments, the self-assembling protein is ferritin. In certain embodiments, the self-assembling protein is added via a sortase A reaction.

In certain aspects, described herein is a nucleic acid encoding any of the recombinant HIV-1 envelope polypeptides described above. In certain aspects, described herein is an immunogenic composition comprising nucleic acid encoding the recombinant HIV-1 envelope described above and a carrier. In certain embodiments, the recombinant HIV-1 envelope is in the form of a trimer, and/or nanoparticle. In certain embodiments, the immunogenic composition further comprises an adjuvant. In certain embodiments, the nucleic acid is operably linked to a promoter, and wherein optionally the nucleic acid is inserted in an expression vector. In certain embodiments, A composition comprising the nucleic acid and a carrier.

In certain aspects, described herein is a recombinant trimer comprising three identical protomers of an envelope from Table 1, Table 3, FIGS. 21-24. In certain aspects, described herein is an immunogenic composition comprising the recombinant trimer from Table 1, Table 3, FIGS. 21-24 and a carrier, wherein the trimer comprises three identical protomers of an HIV-1 envelope listed in Table 1, Table 3, FIGS. 21-24.

In certain aspects, described herein is a method of inducing an immune response in a subject comprising administering in an amount sufficient to affect such induction a recombinant HIV-1 envelope polypeptide, nucleic acid, or immunogenic composition described above in an amount sufficient to induce an immune response. In certain embodiments, the nucleic acid encodes a gp120 envelope, gp120D8 envelope, a gp140 envelope (gp140C, gp140CF, gp140CFI) as soluble or stabilized protomer of a SOSIP trimer, a gp145 envelope, a gp150 envelope, a transmembrane bound envelope, a gp160 envelope or an envelope designed to multimerize. In certain embodiments, the recombinant HIV-1 envelope polypeptide is gp120 envelope, gp120D8 envelope, a gp140 envelope (gp140C, gp140CF, gp140CFI) as soluble or stabilized protomer of a SOSIP trimer, a gp145 envelope, a gp150 envelope, a transmembrane bound envelope, or an envelope designed to multimerize. In certain embodiments, the composition further comprises an adjuvant. In certain embodiments, the method further comprises administering an agent which modulates host immune tolerance. In certain embodiments, the polypeptide administered is multimerized in a liposome or nanoparticle. In certain embodiments, the method further comprises administering one or more additional HIV-1 immunogens to induce a T cell response.

In certain aspects, described herein is a composition comprising a nanoparticle and a carrier, wherein the nanoparticle comprises any one of the recombinant HIV-1 envelopes described above. In certain embodiments, the nanoparticle is ferritin self-assembling nanoparticle.

In certain aspects, described herein is a composition comprising a nanoparticle and a carrier, wherein the nanoparticle comprises any one of the trimers described above. In certain embodiments, the nanoparticle is ferritin self-assembling nanoparticle. In certain embodiments, the nanoparticle comprises multimers of trimers. In certain embodiments, the nanoparticle comprises 1-8 trimers.

In certain aspects, described herein is a method of inducing an immune response in a subject comprising administering in an amount sufficient to affect such induction an immunogenic composition comprising any one of the recombinant HIV-1 envelopes described above or compositions described above. In certain embodiments, the composition is administered as a prime. In certain embodiments, the composition is administered as a boost.

In certain aspects, described herein is a method of inducing an immune response in a subject comprising administering in an amount sufficient to affect such induction an immunogenic composition comprising the nucleic acid described above or the composition described above.

In certain aspects, described herein is a composition comprising the nucleic acid described above and a carrier. In certain embodiments, the nanoparticle comprises any one of the nucleic acids described above. In certain embodiments, the nanoparticle is a lipid nanoparticle.

In certain embodiments, described herein is the recombinant HIV-1 envelope polypeptide, nucleic acid, method, immunogenic composition, or composition described above wherein the HIV-1 envelope is CH505.w24.e5 F14.SOS.GSA.L.Y712I.I535M.A316W.S306L.R308L gp160_mVHss.

### BRIEF DESCRIPTION OF THE DRAWINGS

The patent or application file contains at least one drawing executed in color. To conform to the requirements for PCT patent applications, some figures presented herein are black and white representations of images originally created in color.

FIG. 1 depicts CH235 unmutated common ancestor neutralization of HIV-1 infection of TZM-bl cells. Neutralization was detected against the M5(N279K).G458Y.N197D virus.

FIGS. 2A-B. FIG. 2A depicts positions of the N197D glycan (magenta) relative to the CH235.12 (green) epitope. FIG. 2B depicts biolayer interferometry binding magnitude for CH235 unmutated common ancestor (UCA) IgG and N279K.G458Y.N197D SOSIP gp140 envelope.

FIG. 3 depicts CH235 UCA VH+/−, VL+/− prime-boost studies. CH235 unmutated common ancestor knock-in mouse immunization regimen comparing immunization with Man₅GlcNAc₂-enriched M5.G458Y and M5.G458Y.N197D chimeric SOSIP gp140 envelopes. Envelopes were adjuvanted with 3M-052-Alum. Negative control mice received adjuvant only.

FIG. 4 depicts vaccine induction of trimeric envelope-specific IgG in serum. Post vaccination mouse serum IgG binding to HIV-1 envelope SOSIP gp140 trimers as measured by ELISA. Each symbol represents an individual mouse. Lines indicate the group means.

FIG. 5 depicts group comparison of vaccine induction of trimeric envelope-specific IgG in serum. Post vaccination mouse serum IgG binding to HIV-1 envelope SOSIP gp140 trimers as measured by ELISA. Each symbol represents an individual mouse. Lines indicate the group means.

FIG. 6 depicts the lack of vaccine induction of antibodies to non-neutralizing epitopes in V2, V3, or gp41. Post vaccination mouse serum IgG binding to HIV-1 envelope linear peptide and gp41 as measured by ELISA. Each symbol represents an individual mouse. Lines indicate the group means.

FIG. 7 depicts Mu509 serum neutralization. Post vaccination mouse serum neutralization of HIV-1 infection of TZM-bl cells. Neutralization titer is shown as reciprocal dilution of serum that inhibits 50% of virus replication. Values for individual mice and group geometric means are shown.

FIG. 8 depicts human VH somatic mutation frequency determined by next generation sequencing of splenic B cells.

FIG. 9 depicts mouse VH somatic mutation frequency determined by next generation sequencing of splenic B cells.

FIG. 10 depicts human VK somatic mutation frequency determined by next generation sequencing of splenic B cells.

FIG. 11 depicts mouse VK somatic mutation frequency determined by next generation sequencing of splenic B cells.

FIG. 12 depicts the percentage of VH or VK sequences derived from human knock-in genes. Group medians are shown by black bars.

FIG. 13 depicts the frequency of occurrence of individual amino acid substitutions in CH235 antibodies after vaccination. Induction of somatic mutations indicates initiation of affinity maturation of the CH235 antibodies. Adjuvant only control mice showed minimal induction somatic mutation compared to envelope plus adjuvant groups.

FIG. 14 depicts the frequency of occurrence of individual amino acid substitutions in CH235 antibodies after vaccination. Induction of somatic mutations indicates initiation of affinity maturation of the CH235 antibodies. Adjuvant only control mice showed minimal induction somatic mutation compared to envelope plus adjuvant groups.

FIG. 15 depicts the frequency of occurrence of individual amino acid substitutions in CH235 antibodies after vaccination. Induction of somatic mutations indicates initiation of affinity maturation of the CH235 antibodies. Adjuvant only control mice showed minimal induction somatic mutation compared to envelope plus adjuvant groups.

FIGS. 16A-E depict LOGO plots of amino acid substitutions in CH235 VH gene segments observed in each mouse (mouse numbers 50902-50906) after vaccination with CH505.M5.G458Y.N197D stabilized gp140 plus 3M-052.

FIGS. 17A-J depict LOGO plots. FIGS. 17A-E depict LOGO plots of amino acid substitutions in CH235 VH gene segments observed in each mouse (mouse numbers 50908-50912) after vaccination with CH505.M5.G458Y Man₅GlcNAc₂-enriched stabilized gp140 plus 3M-052. FIGS. 17F-J depict LOGO plots of amino acid substitutions in CH235 VH gene segments observed in each mouse (mouse numbers 50913-50918) after vaccination with 3M-052 only.

FIGS. 18A-F depict LOGO plots of amino acid substitutions in CH235 VK gene segments observed in each mouse (mouse numbers 50901-50906) after vaccination with CH505.M5.G458Y.N197D stabilized gp140 plus 3M-052.

FIGS. 19A-F depict LOGO plots of amino acid substitutions in CH235 VK gene segments observed in each mouse (mouse numbers 50907-50912) after vaccination with CH505.M5.G458Y Man₅GlcNAc₂-enriched stabilized gp140 plus 3M-052.

FIGS. 20A-F depict LOGO plots of amino acid substitutions in CH235 VK gene segments observed in each mouse (mouse numbers 50913-50918) after vaccination with 3M-052-alum only.

FIGS. 21A-B show non-limiting embodiments of a nucleic acid sequence (FIG. 21A) and an amino acid sequence (FIG. 21B) for HV1301288_G458Y_N197D. The signal peptide is underlined.

FIGS. 22A-FF show the amino acid sequences of Table 1, wherein the sequences are annotated to show envelope portion, linker, ferritin, and other elements. For nanoparticle fusion envelopes the N-terminal signal peptide is bold and underlined, the amino acids immediately following the signal peptide up to the linker represents the HIV-1 envelope, the linker is shown in gray and is outlined with a double lined box and represents the linker used to fuse the envelope to the ferritin subunit, the ferritin subunit is the amino acids immediately following the linker to the C-terminus. Signal peptide is underlined and is MPMGSLQPLATLYLLGMLVASVLA or MGWSCIILFLVATATGVHA. In these amino acid sequences, F14 stabilization amino acids are shown in boxes but are not italic letters. Italic letters in rectangles are interprotomer contacts formed within a single trimeric envelope. I535M is the only interprotomer contact that was changed. Underlined Y signifies the G458Y substitution. Underlined K denotes M5 or N279K substitution. Underlined Q indicates N19Q ferritin substitution that removes glycosylation sequence. Underlined D is the N197D substitution.

FIG. 23 shows the nucleic acid sequences of Table 1.

FIGS. 24A-B show the nucleic acid sequences (FIG. 24A) and the amino acid sequences (FIG. 24B) of Table 3. For nanoparticle fusion envelopes the N-terminal signal peptide is bold underlined and/or is MPMGSLQPLATLYLLGMLVASVLA or MGWSCIILFLVATATGVHA or MDAMKRGLCCVLLLCGAVFVSPSQEIHARFRRGAR. In these amino acid sequences, F14 stabilization amino acids (A204V, V208L, V68I, and V255L), SOS substitutions (A501C and T605C), I535M, A316W, Y712I, where present may be shown in boxes. I535M is the only interprotomer contact that was changed.

FIG. 25 shows the mRNA sequences of Table 4.

### DETAILED DESCRIPTION OF THE INVENTION

The development of a safe, highly efficacious prophylactic HIV-1 vaccine is of paramount importance for the control and prevention of HIV-1 infection. A major goal of HIV-1 vaccine development is the induction of broadly neutralizing antibodies (bnAbs) (Immunol. Rev. 254: 225-244, 2013). BnAbs are protective in rhesus macaques against SHIV challenge, but as yet, are not induced by current vaccines.

For the past 25 years, the HIV vaccine development field has used single or prime boost heterologous Envs as immunogens, but to date has not found a regimen to induce high levels of bnAbs.

Recently, a new paradigm for design of strategies for induction of broadly neutralizing antibodies was introduced, that of B cell lineage immunogen design (Nature Biotech. 30: 423, 2012) in which the induction of bnAb lineages is recreated. It was recently demonstrated the power of mapping the co-evolution of bnAbs and founder virus for elucidating the Env evolution pathways that lead to bnAb induction (Nature 496: 469, 2013).

### Sequences/Clones

Described herein are nucleic and amino acids sequences of HIV-1 envelopes. The sequences for use as immunogens are in any suitable form. In certain embodiments, the described HIV-1 envelope sequences are gp160s. In certain embodiments, the described HIV-1 envelope sequences are gp120s. Other sequences, for example but not limited to stable SOSIP trimer designs, gp145s, gp140s, both cleaved and uncleaved, gp140 Envs with the deletion of the cleavage (C) site, fusion (F) and immunodominant (I) region in gp41—named as gp140ΔCFI (gp140CFI), gp140 Envs with the deletion of only the cleavage (C) site and fusion (F) domain—named as gp140ΔCF (gp140CF), gp140 Envs with the deletion of only the cleavage (C)—named gp140ΔC (gp140C) (See e.g., Liao et al. Virology 2006, 353, 268-282), gp150s, gp41s, which are readily derived from the nucleic acid and amino acid gp160 sequences. In certain embodiments the nucleic acid sequences are codon optimized for optimal expression in a host cell, for example a mammalian cell, a rBCG cell or any other suitable expression system.

An HIV-1 envelope has various structurally defined fragments/forms: gp160; gp140—including cleaved gp140 and uncleaved gp140 (gp140C), gp140CF, or gp140CFI; gp120 and gp4l. A skilled artisan appreciates that these fragments/forms are defined not necessarily by their crystal structure, but by their design and bounds within the full length of the gp160 envelope. While the specific consecutive amino acid sequences of envelopes from different strains are different, the bounds and design of these forms are well known and characterized in the art.

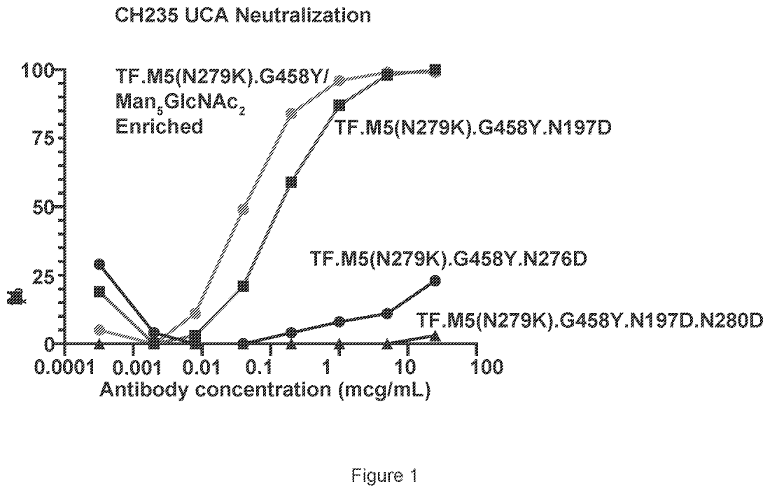

For example, it is well known in the art that during its transport to the cell surface, the gp160 polypeptide is processed and proteolytically cleaved to gp120 and gp41 proteins. Cleavages of gp160 to gp120 and gp41 occurs at a conserved cleavage site “REKR.” See Chakrabarti et al. Journal of Virology vol. 76, pp. 5357-5368 (2002) see for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353(2): 268-282 (2006).

The role of the furin cleavage site was well understood both in terms of improving cleave efficiency, see Binley et al. supra, and eliminating cleavage, see Bosch and Pawlita, Virology 64 (5):2337-2344 (1990); Guo et al. Virology 174: 217-224 (1990); McCune et al. Cell 53:55-67 (1988); Liao et al. J Virol. April; 87(8):4185-201 (2013).

Likewise, the design of gp140 envelope forms is also well known in the art, along with the various specific changes which give rise to the gp140C (uncleaved envelope), gp140CF and gp140CFI forms. Envelope gp140 forms are designed by introducing a stop codon within the gp41 sequence. See Chakrabarti et al. at FIG. 1.

Envelope gp140C refers to a gp140 HIV-1 envelope design with a functional deletion of the cleavage (C) site, so that the gp140 envelope is not cleaved at the furin cleavage site. The specification describes cleaved and uncleaved forms, and various furin cleavage site modifications that prevent envelope cleavage are known in the art. In some embodiments of the gp140C form, two of the R residues in and near the furin cleavage site are changed to E, e.g., RRVVEREKR is changed to ERVVEREKE, and is one example of an uncleaved gp140 form. Another example is the gp140C form which has the REKR site changed to SEKS. See supra for references.

Envelope gp140CF refers to a gp140 HIV-1 envelope design with a deletion of the cleavage (C) site and fusion (F) region. Envelope gp140CFI refers to a gp140 HIV-1 envelope design with a deletion of the cleavage (C) site, fusion (F) and immunodominant (I) region in gp41. See Chakrabarti et al. Journal of Virology vol. 76, pp. 5357-5368 (2002) see for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353(2): 268-282 (2006).

In certain embodiments, the envelope design in accordance with the present invention involves deletion of residues (e.g., 5-11, 5, 6, 7, 8, 9, 10, or 11 amino acids) at the N-terminus. For delta N-terminal design, amino acid residues ranging from 4 residues or even fewer to 14 residues or even more are deleted. These residues are between the maturation (signal peptide, usually ending with CX, X can be any amino acid) and “VPVXXXX . . . ”. In case of CH505 T/F Env as an example, 8 amino acids (italicized and underlined in the below sequence) were deleted:

MRVMGIQRNYPQWWIWSMLGFWMLMICNGMWVTVYYGVPVWKEAKTTLFCASDA
KAYEKEVHNVWATHACVPTDPNPQE...(rest of envelope sequence is indicated as “...”).

In other embodiments, the delta N-design described for CH505 T/F envelope can be used to make delta N-designs of other CH505 envelopes. In certain embodiments, the invention relates generally to an immunogen, gp160, gp120 or gp140, without an N-terminal Herpes Simplex gD tag substituted for amino acids of the N-terminus of gp120, with an HIV leader sequence (or other leader sequence), and without the original about 4 to about 25, for example 4, 5, 6, 7, 8, 9, 10, 11, 12, 13, 14, 15, 16, 17, 18, 19, 20, 21, 22, 23, 24, 25 amino acids of the N-terminus of the envelope (e.g. gp120). See U.S. patent Ser. No. 10/040,826, e.g. at 5:26-6:67, the contents of which publication is hereby incorporated by reference in its entirety.

The general strategy of deletion of N-terminal amino acids of envelopes results in proteins, for example gp120s, expressed in mammalian cells that are primarily monomeric, as opposed to dimeric, and, therefore, solves the production and scalability problem of commercial gp120 Env vaccine production. In other embodiments, the amino acid deletions at the N-terminus result in increased immunogenicity of the envelopes.

In certain aspects, the invention provides composition and methods which CH505 Envs, as gp120s, gp140s cleaved and uncleaved, gp145s, gp150s and gp160s, stabilized and/or multimerized trimers, as proteins, DNAs, RNAs, or any combination thereof, administered as primes and boosts to elicit immune response. CH505 envelopes as proteins would be co-administered with nucleic acid vectors encoding the envelopes to amplify antibody induction. In certain embodiments, the compositions and methods include any immunogenic HIV-1 sequences to give the best coverage for T cell help and cytotoxic T cell induction. In certain embodiments, the compositions and methods include mosaic and/or consensus HIV-1 genes to give the best coverage for T cell help and cytotoxic T cell induction. In certain embodiments, the compositions and methods include mosaic group M and/or consensus genes to give the best coverage for T cell help and cytotoxic T cell induction. In some embodiments, the mosaic genes are any suitable gene from the HIV-1 genome. In some embodiments, the mosaic genes are Env genes, Gag genes, Pol genes, Nef genes, or any combination thereof. See e.g. U.S. Pat. No. 7,951,377. In some embodiments the mosaic genes are bivalent mosaics. In some embodiments the mosaic genes are trivalent. In some embodiments, the mosaic genes are administered in a suitable vector with each immunization with Env gene inserts in a suitable vector and/or as a protein. In some embodiments, the mosaic genes, for example as bivalent mosaic Gag group M consensus genes, are administered in a suitable vector, for example but not limited to HSV2, would be administered with each immunization with Env gene inserts in a suitable vector, for example but not limited to HSV-2.

### Nucleic Acid Sequences

In certain aspects the invention provides compositions and methods of Env genetic immunization either alone or with Env proteins to recreate the swarms of evolved viruses that have led to bnAb induction. Nucleotide-based vaccines offer a flexible vector format to immunize against virtually any protein antigen. Currently, two types of genetic vaccination are available for testing—DNAs and mRNAs.

In certain aspects the invention contemplates using immunogenic compositions wherein immunogens are delivered as DNA. See Graham B S, Enama M E, Nason M C, Gordon I J, Peel S A, et al. (2013) DNA Vaccine Delivered by a Needle-Free Injection Device Improves Potency of Priming for Antibody and CD8+ T-Cell Responses after rAd5 Boost in a Randomized Clinical Trial. PLoS ONE 8(4): e59340, page 9. Various technologies for delivery of nucleic acids, as DNA and/or RNA, so as to elicit immune response, both T-cell and humoral responses, are known in the art and are under developments. In certain embodiments, DNA can be delivered as naked DNA. In certain embodiments, DNA is formulated for delivery by a gene gun. In certain embodiments, DNA is administered by electroporation, or by a needle-free injection technologies, for example but not limited to Biojector® device. In certain embodiments, the DNA is inserted in vectors. The DNA is delivered using a suitable vector for expression in mammalian cells. In certain embodiments the nucleic acids encoding the envelopes are optimized for expression. In certain embodiments DNA is optimized, e.g. codon optimized, for expression. In certain embodiments the nucleic acids are optimized for expression in vectors and/or in mammalian cells. In non-limiting embodiments these are bacterially derived vectors, adenovirus based vectors, rAdenovirus (e.g. Barouch D H, et al. Nature Med. 16: 319-23, 2010), recombinant mycobacteria (e.g. rBCG or M. smegmatis) (Yu, J S et al. Clinical Vaccine Immunol. 14: 886-093,2007; ibid 13: 1204-11, 2006), and recombinant vaccinia type of vectors (Santra S. Nature Med. 16: 324-8, 2010), for example but not limited to ALVAC, replicating (Kibler K V et al., PLoS One 6: e25674, 2011 nov 9.) and non-replicating (Perreau M et al. J. virology 85: 9854-62, 2011) NYVAC, modified vaccinia Ankara (MVA)), adeno-associated virus, Venezuelan equine encephalitis (VEE) replicons, Herpes Simplex Virus vectors, and other suitable vectors.

In certain aspects the invention contemplates using immunogenic compositions wherein immunogens are delivered as DNA or RNA in suitable formulations. Various technologies which contemplate using DNA or RNA or may use complexes of nucleic acid molecules and other entities to be used in immunization. In certain embodiments, DNA or RNA is administered as nanoparticles consisting of low dose antigen-encoding DNA formulated with a block copolymer (amphiphilic block copolymer 704). See Cany et al., Journal of Hepatology 2011 vol. 54 j 115-121; Arnaoty et al., Chapter 17 in Yves Bigot (ed.), Mobile Genetic Elements: Protocols and Genomic Applications, Methods in Molecular Biology, vol. 859, pp 293-305 (2012); Arnaoty et al. (2013) Mol Genet Genomics. 2013 August; 288(7-8):347-63. Nanocarrier technologies called Nanotaxi® for immunogenic macromolecules (DNA, RNA, Protein) delivery are under development. See for example technologies developed by Incellart.

In certain aspects, the invention provides nucleic acids comprising sequences encoding envelopes of the invention. In certain embodiments, the nucleic acids are DNAs. In certain embodiments, the nucleic acids are mRNAs. In certain aspects, the invention provides expression vectors comprising the nucleic acids of the invention.

In certain aspects, the invention provides a pharmaceutical composition comprising mRNAs encoding the inventive antibodies. In certain embodiments, these are optionally formulated in lipid nanoparticles (LNPs). In certain embodiments, the mRNAs are modified. Modifications include without limitations modified ribonucleotides, poly-A tail, 5′cap.

In certain aspects the invention provides nucleic acids encoding the inventive envelopes. In non-limiting embodiments, the nucleic acids are mRNA, modified or unmodified, suitable for use any use, e.g. but not limited to use as pharmaceutical compositions. In certain embodiments, the nucleic acids are formulated in lipid, such as but not limited to LNPs.

In certain aspects, the invention provides nucleic acids comprising sequences encoding proteins of the invention. In certain embodiments, the nucleic acids are DNAs. In certain embodiments, the nucleic acids are mRNAs. In certain aspects, the invention provides expression vectors comprising the nucleic acids of the invention.

In certain aspects, the invention provides a pharmaceutical composition comprising mRNAs encoding the inventive antibodies. In certain embodiments, these are optionally formulated in lipid nanoparticles (LNPs). In certain embodiments, the mRNAs are modified. Modifications include without limitations modified ribonucleotides, poly-A tail, 5′cap.

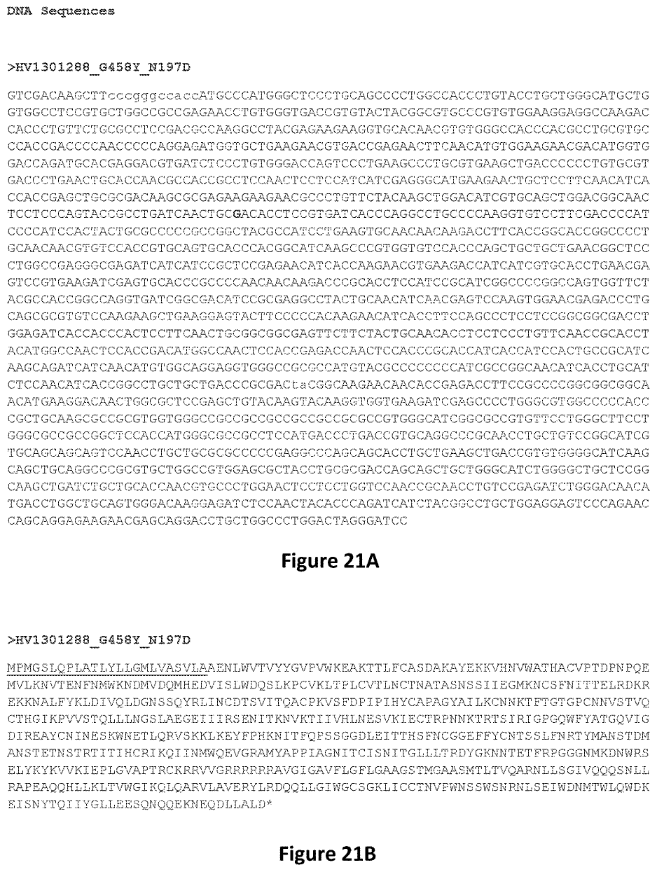

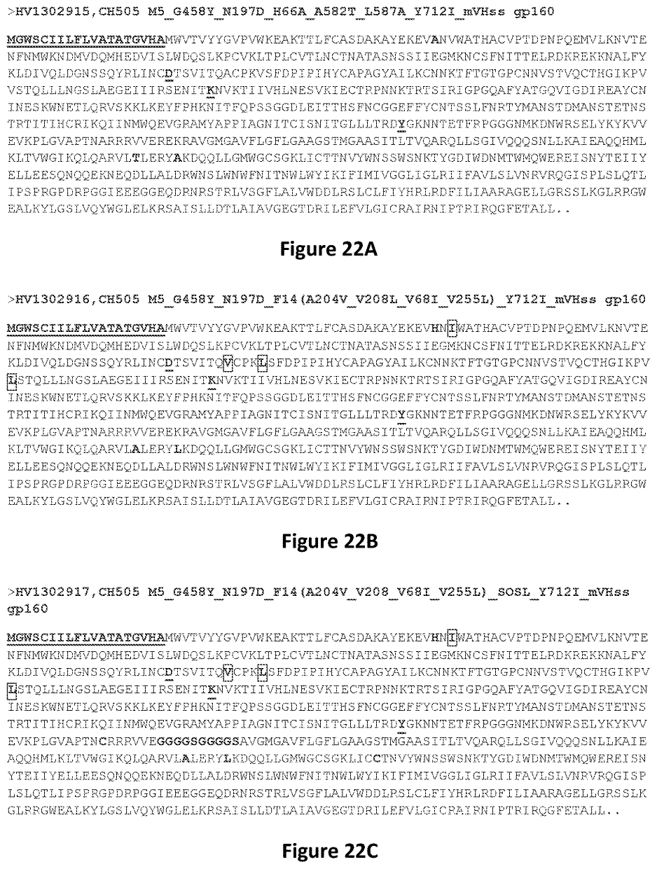

Nucleic acid sequences provided herein, e.g. see FIG. 21A, 23 or 24A, are provided as DNA sequences. However, it should be understood that such sequences also represent RNA sequences, for example, mRNA sequences. For example, RNA polymerase can be used to make RNA sequences from DNA sequences. In RNA sequences, thymine will be uridine. In some embodiments, uridine will be 1-methyl-pseudouridine. In some embodiments, nucleic acids of the invention, including RNA sequences or mRNA sequences, can further comprise any type of modified nucleotides, including, but not limited to 5-methyl-cytidine, 6-methyl-adenosine, or modified uridine.

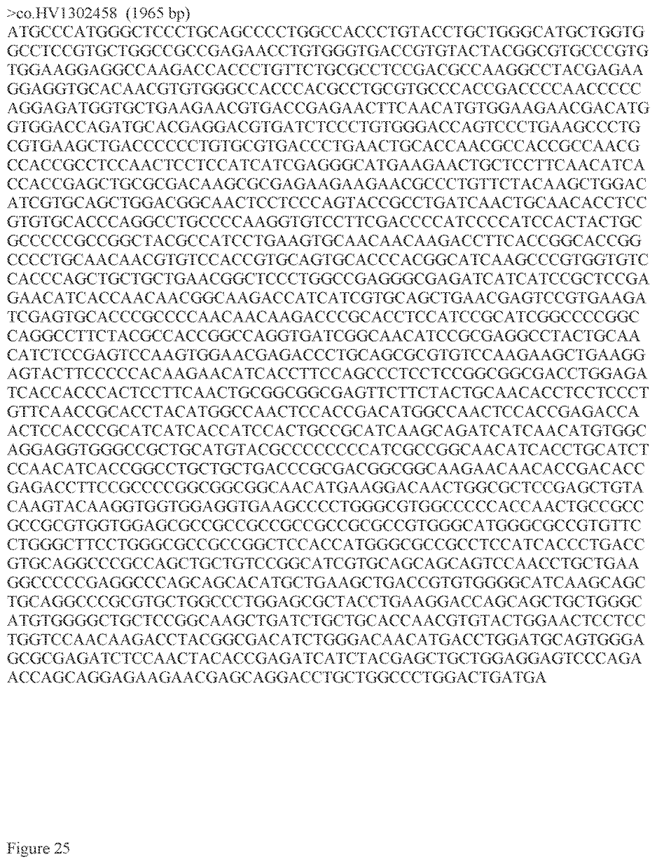

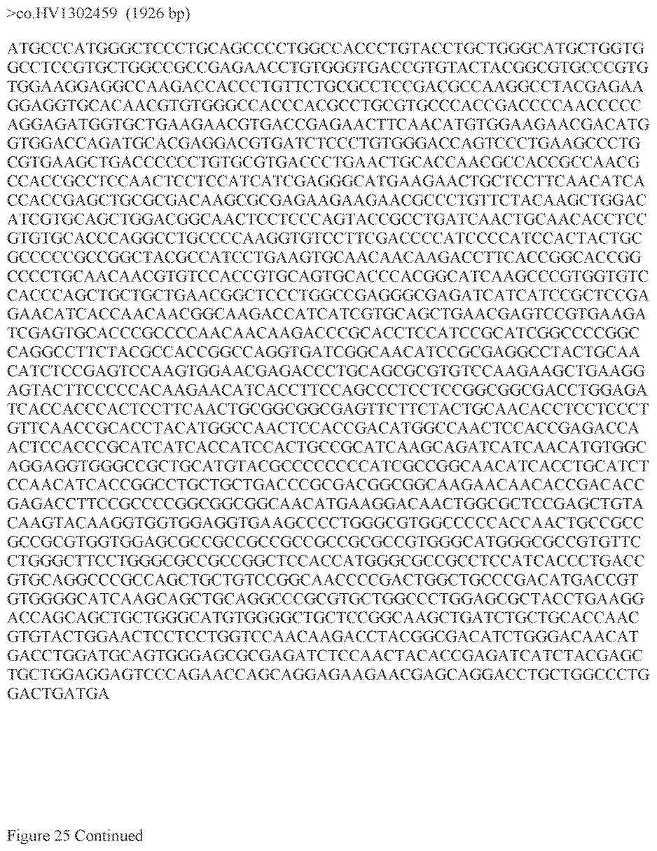

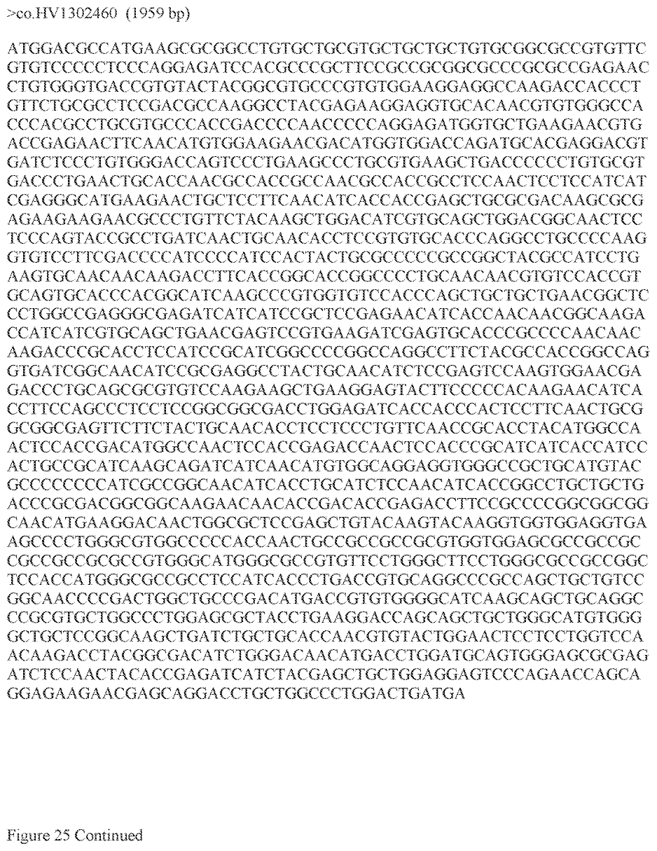

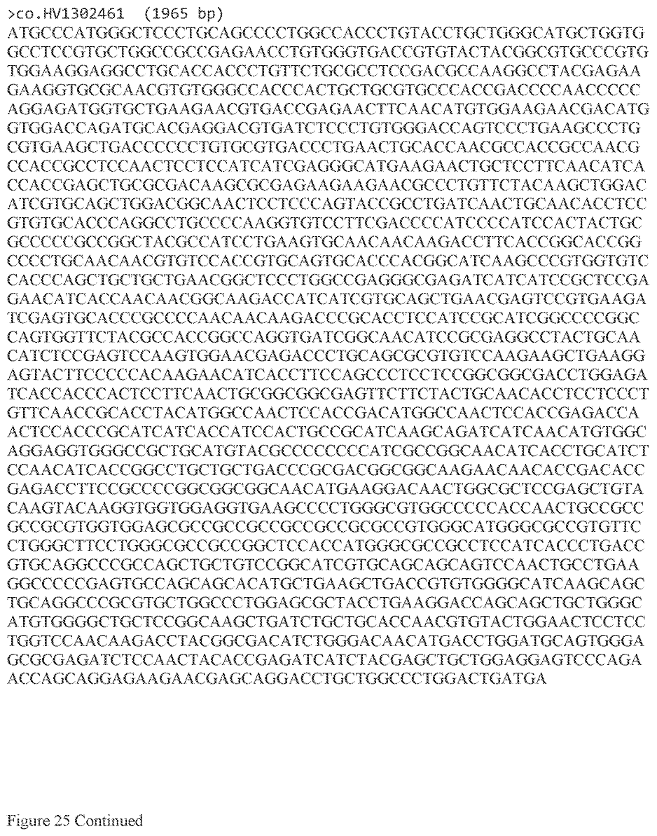

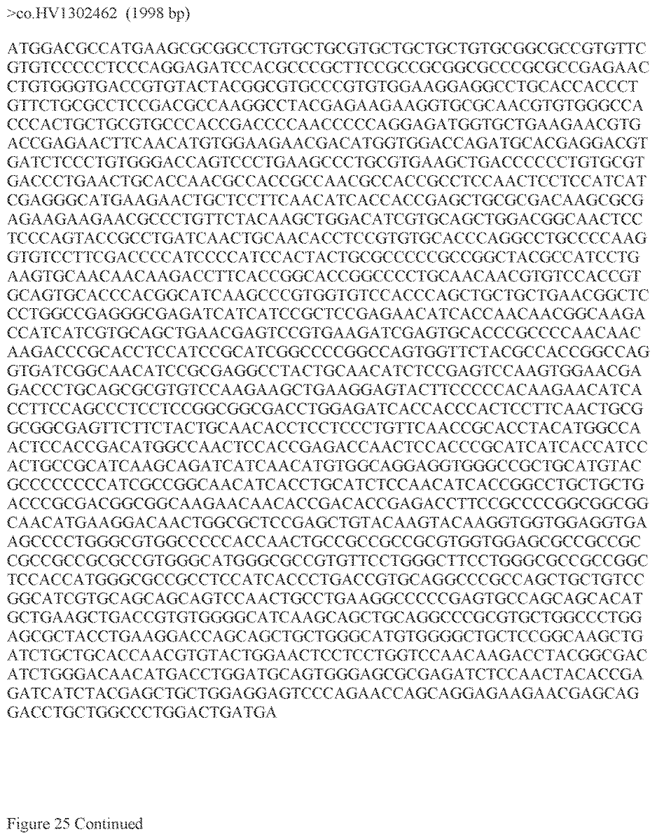

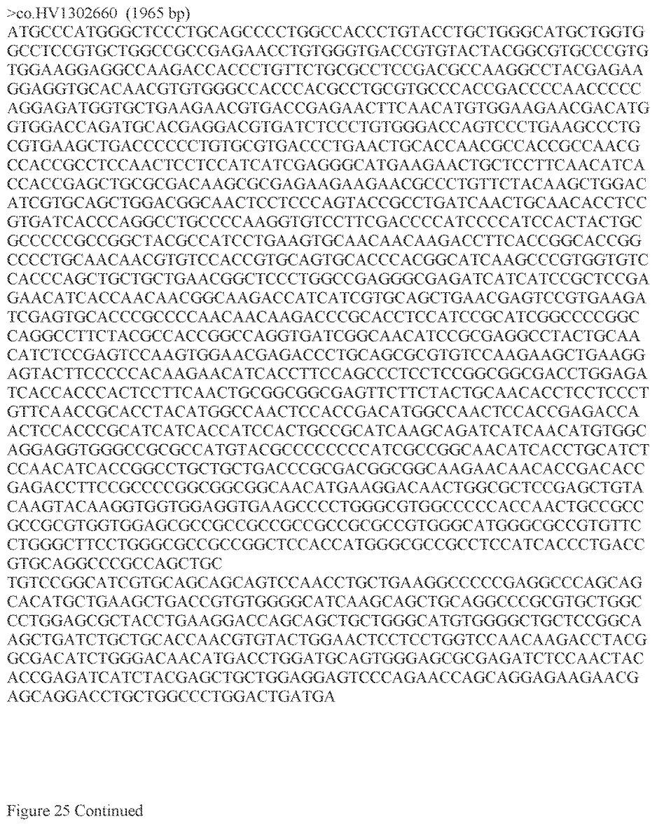

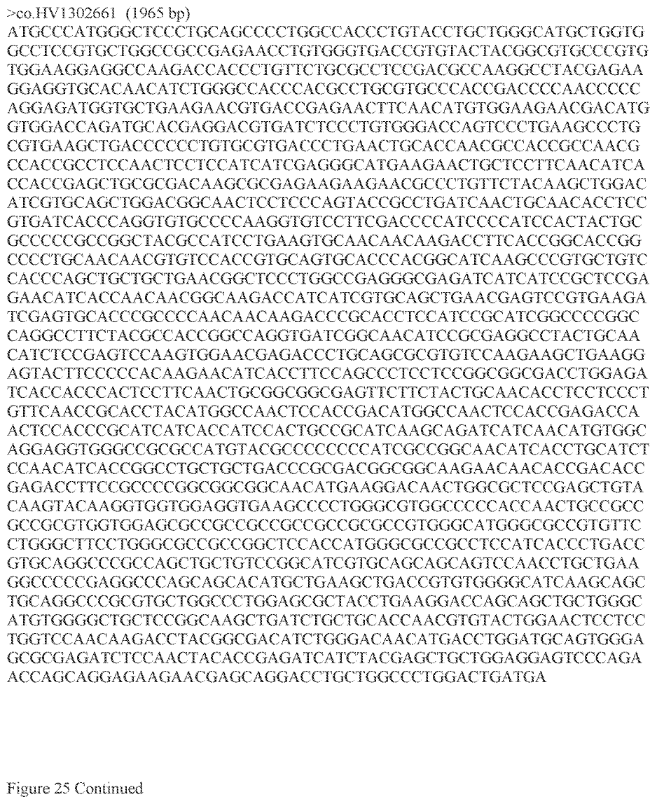

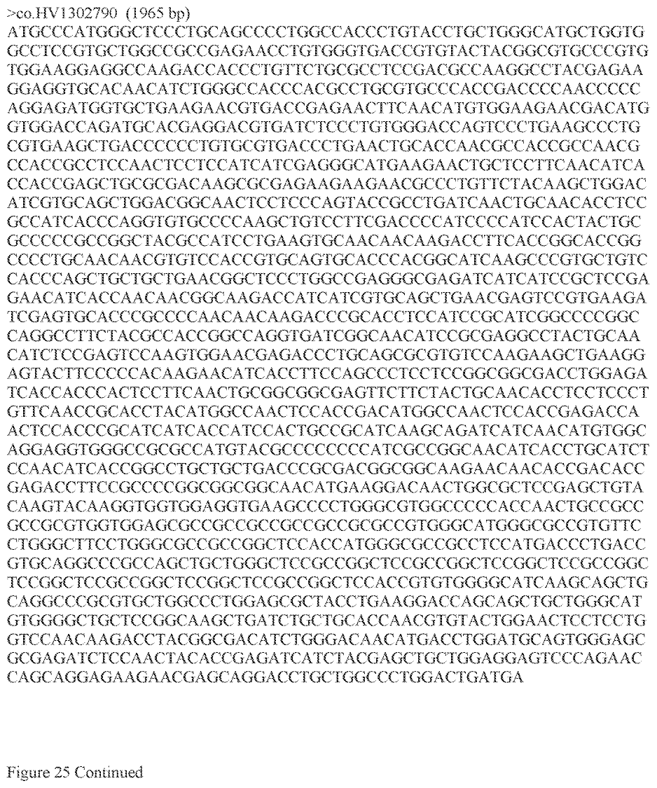

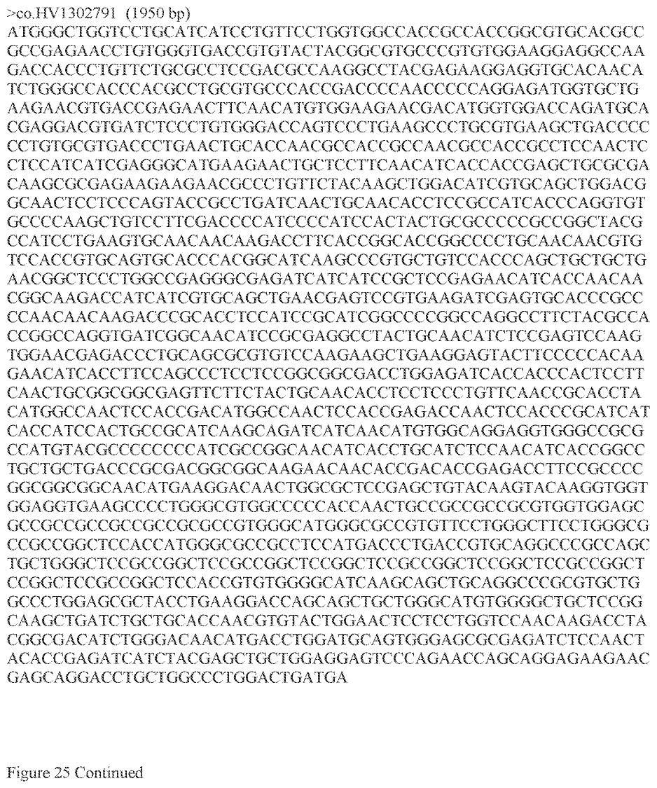

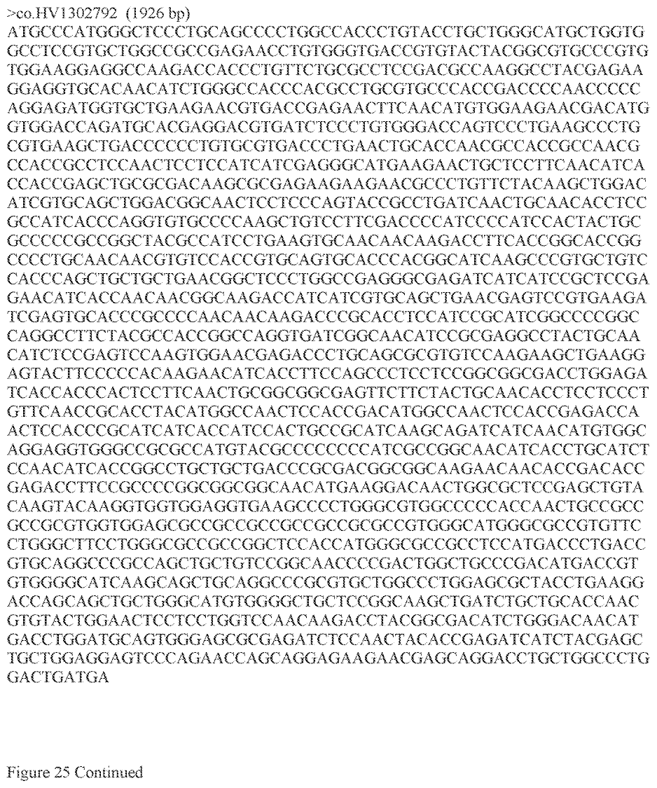

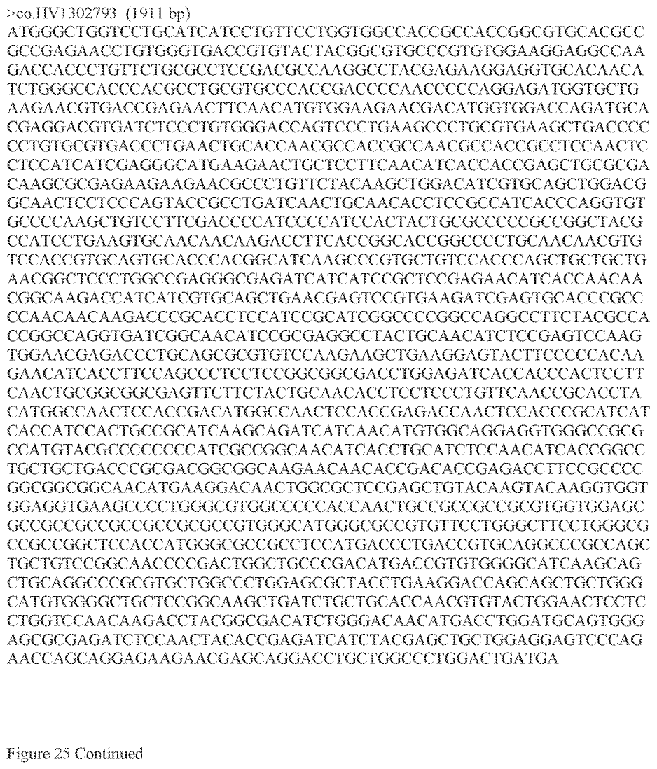

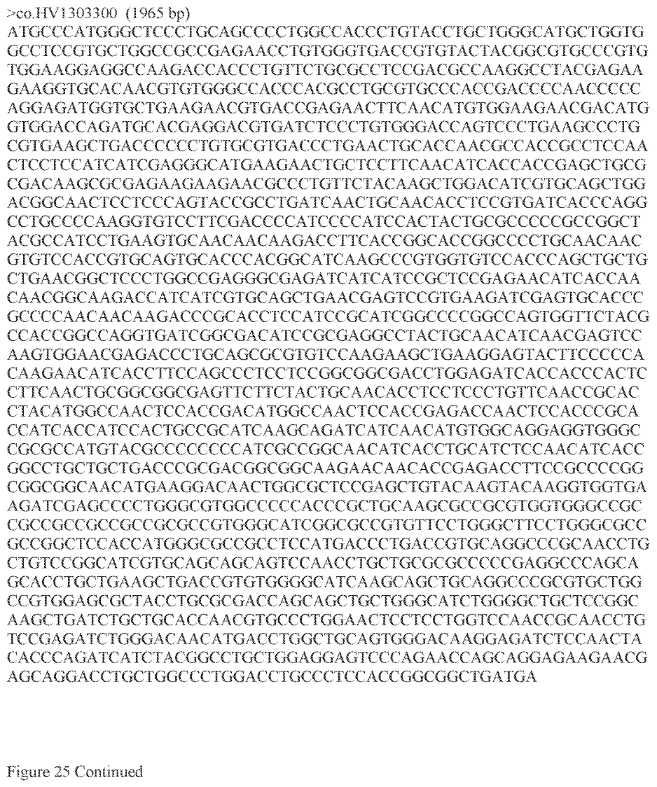

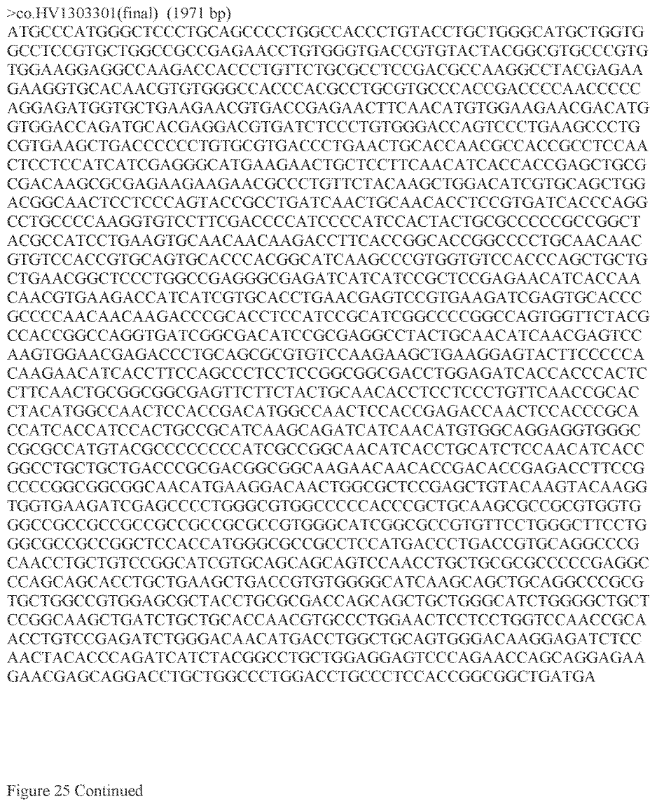

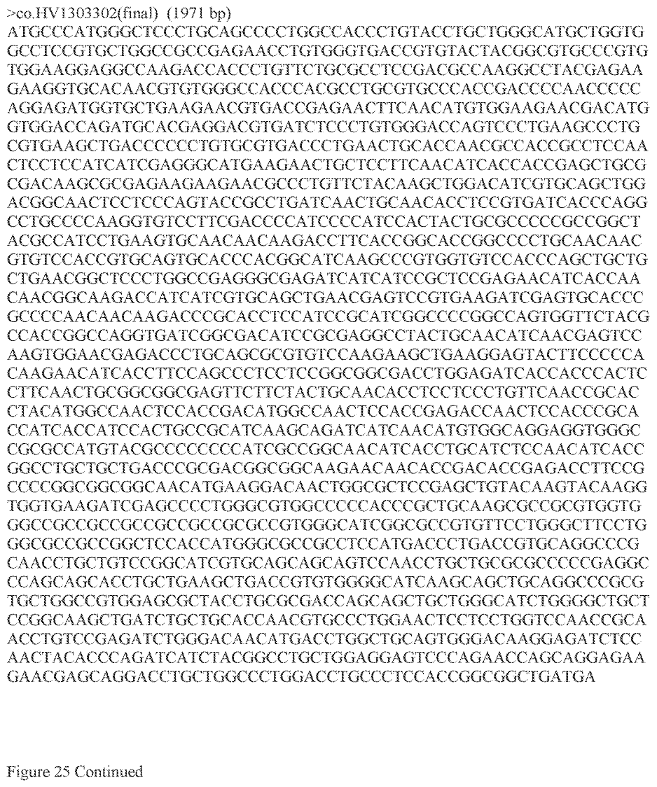

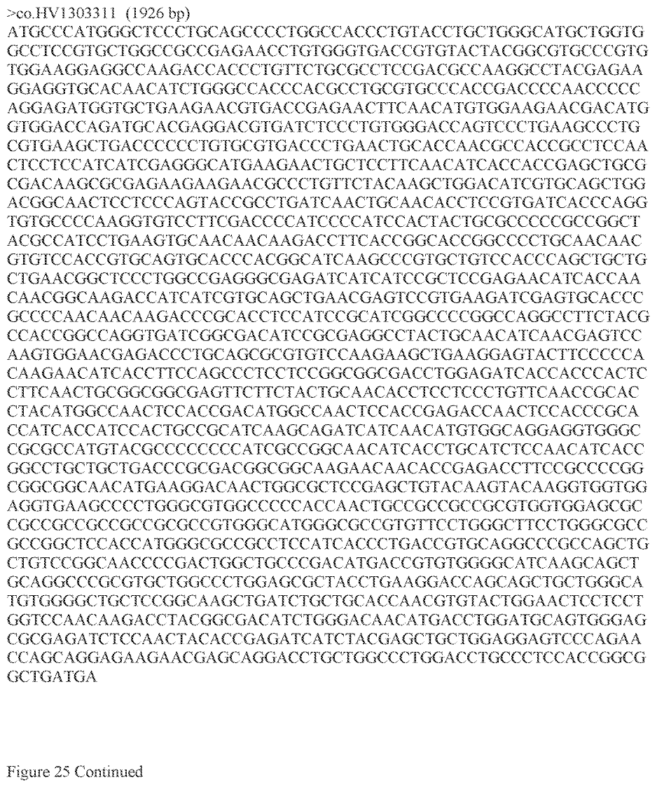

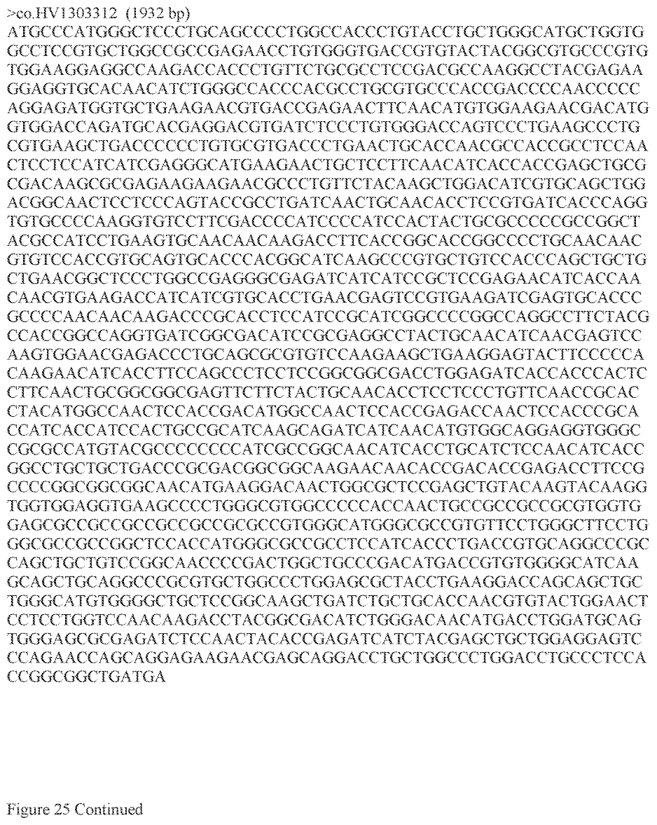

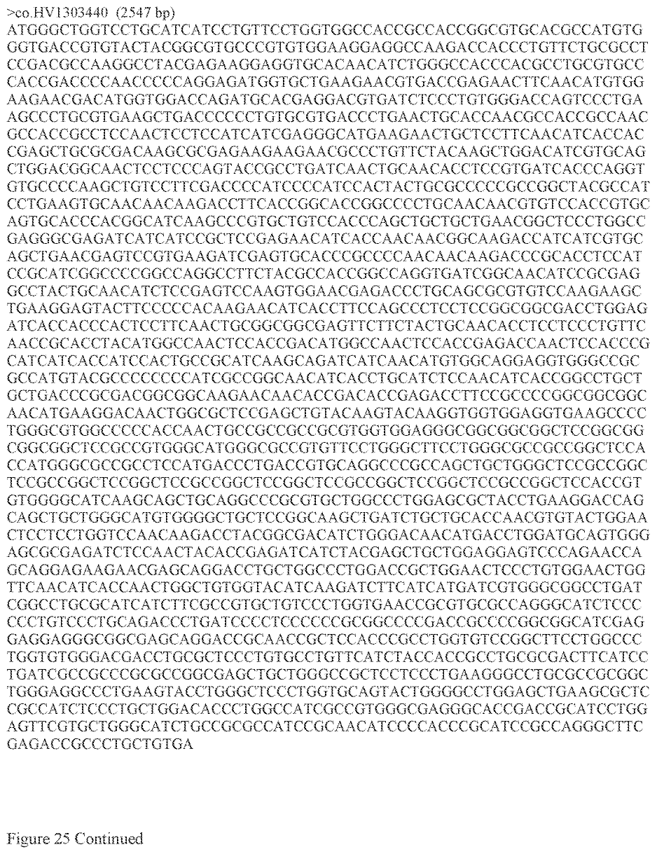

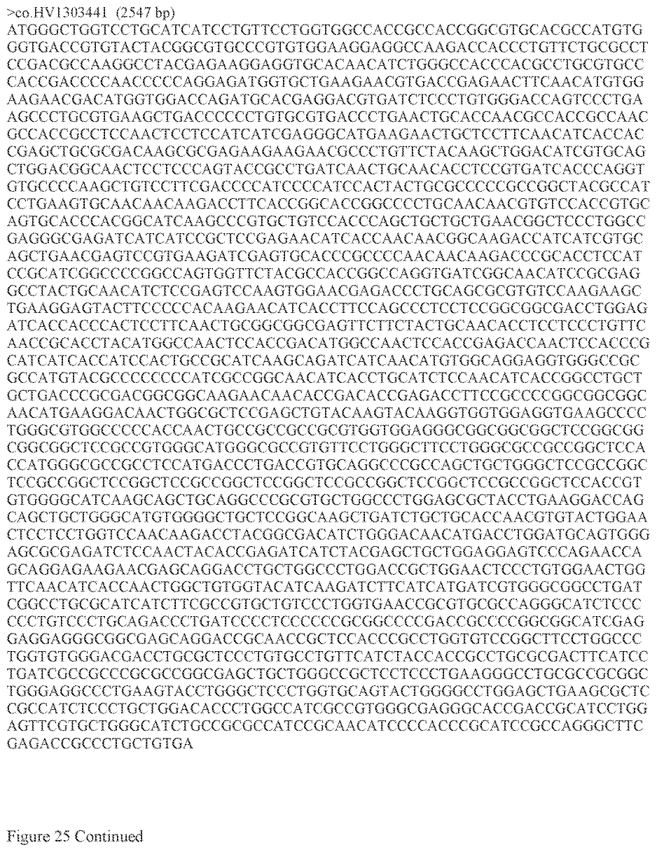

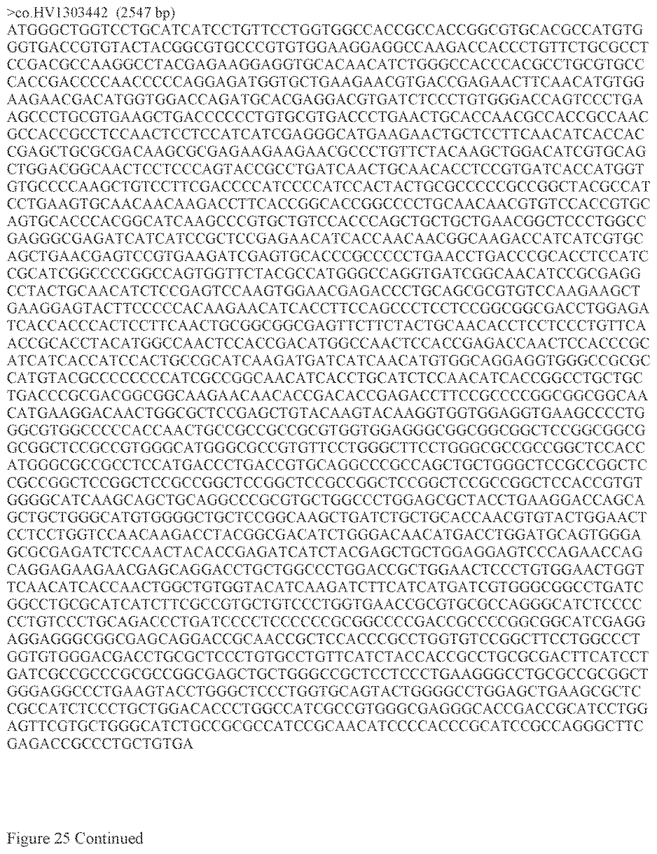

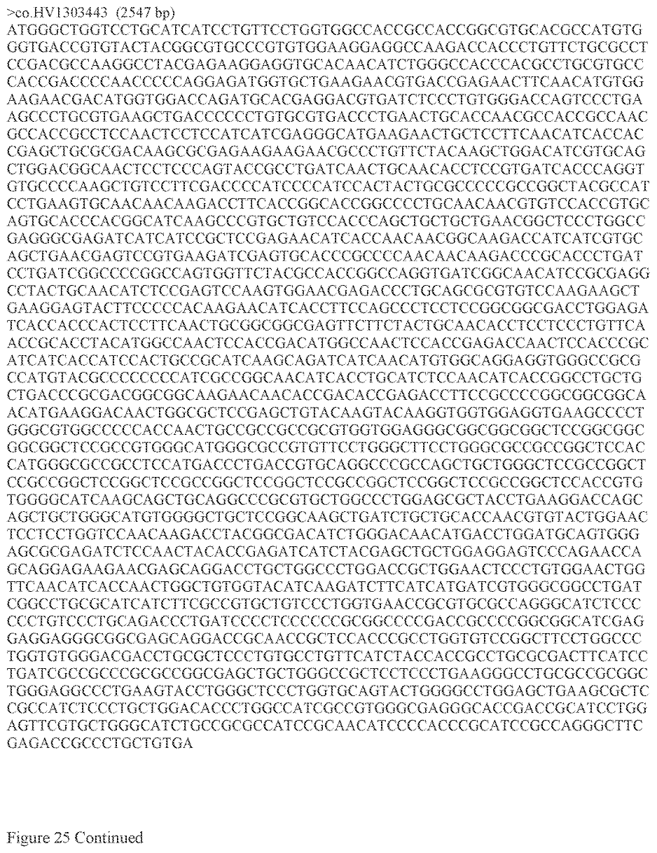

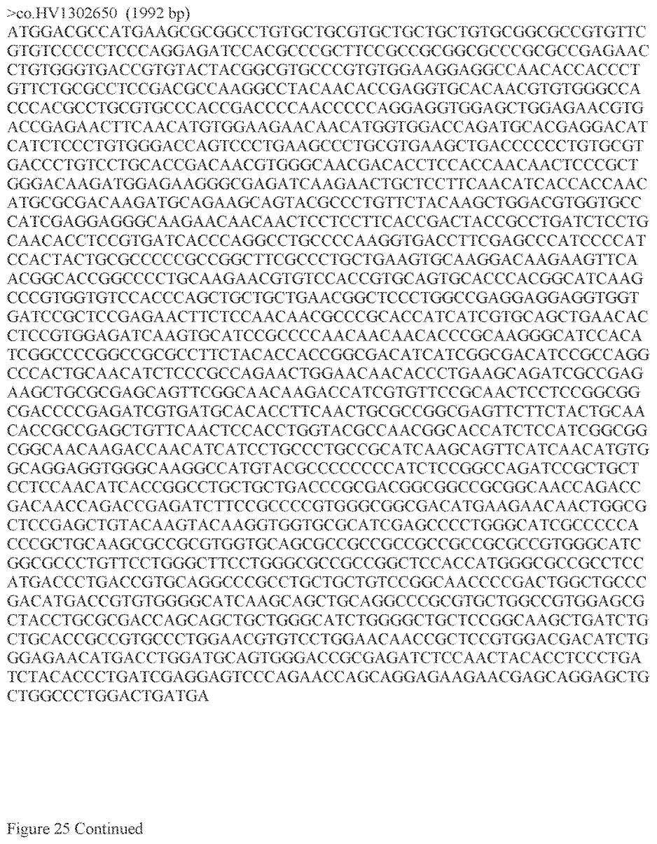

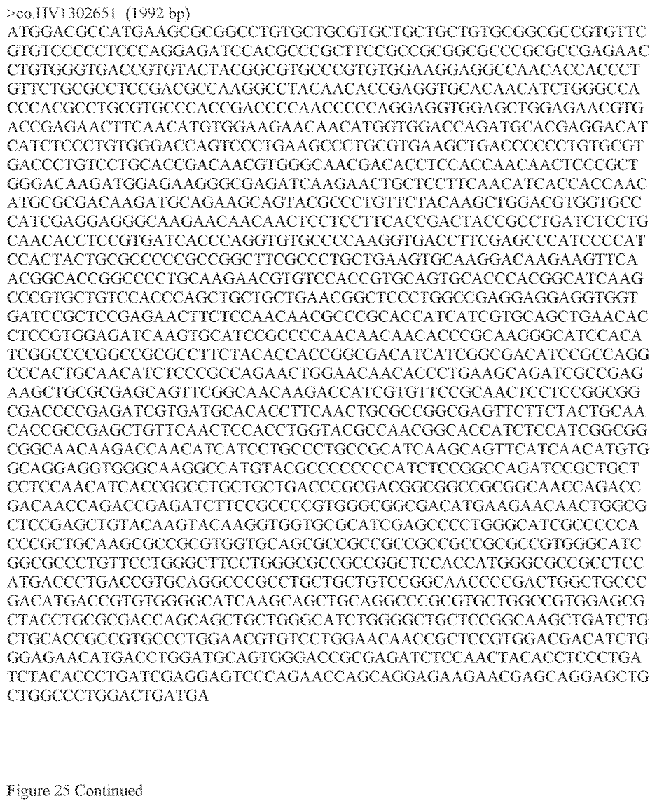

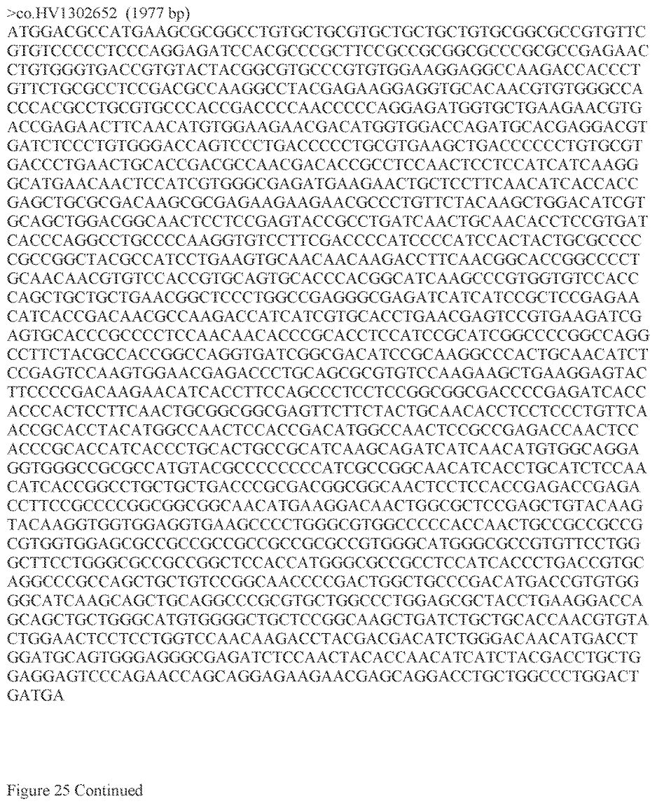

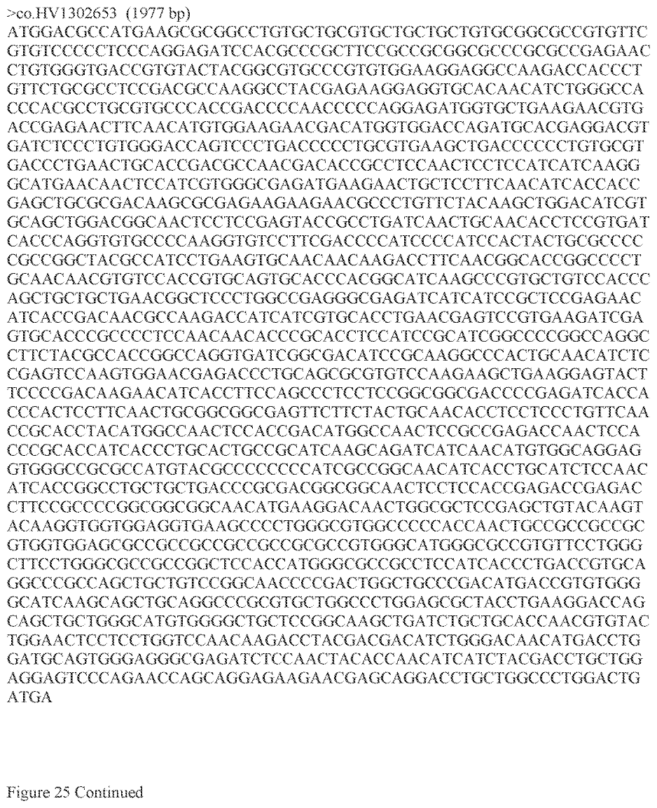

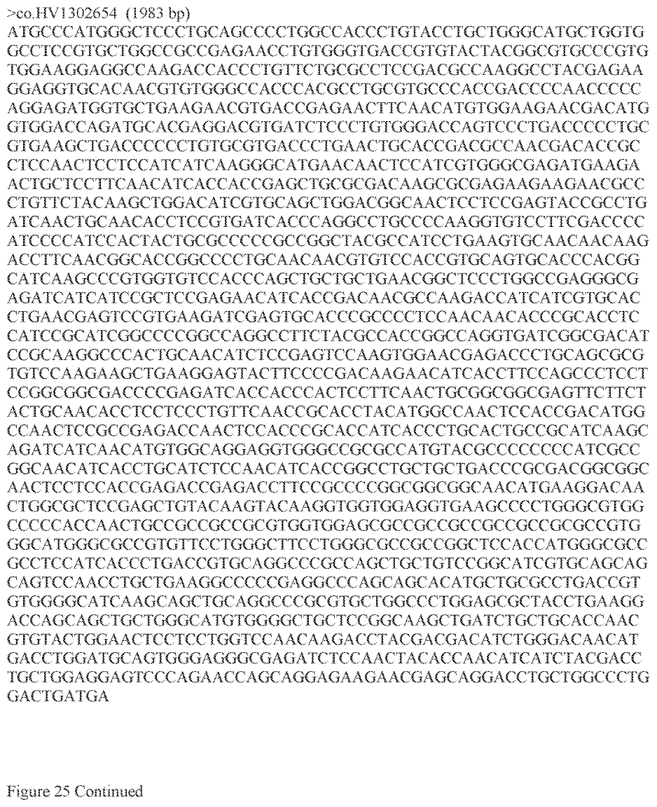

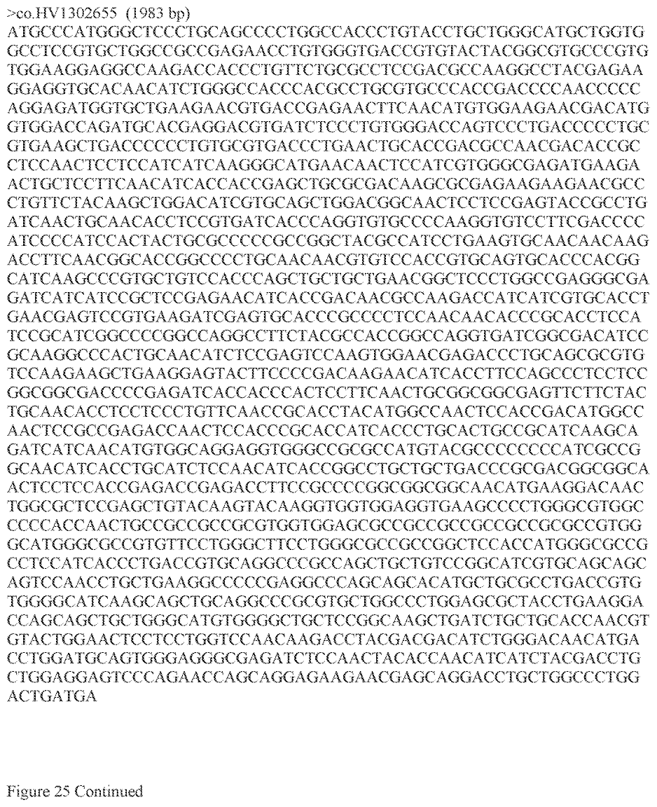

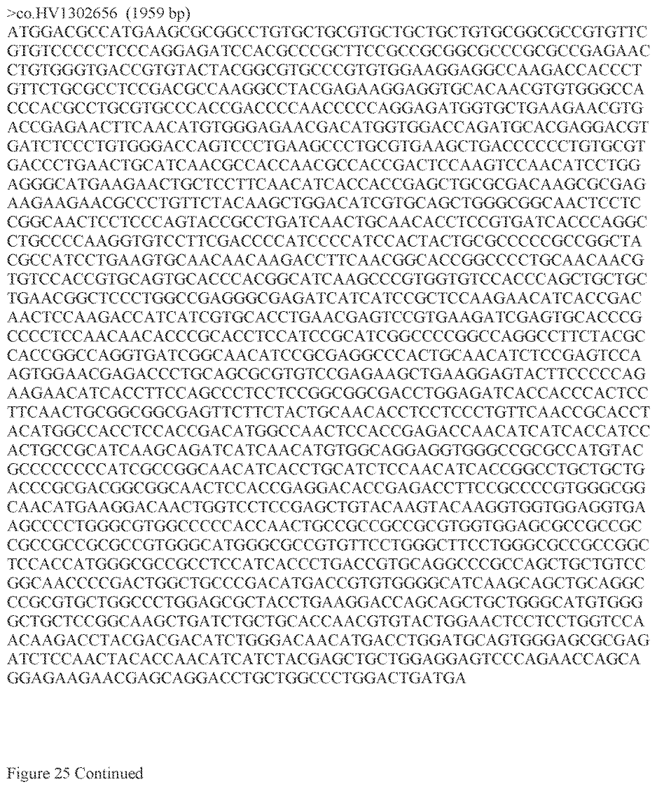

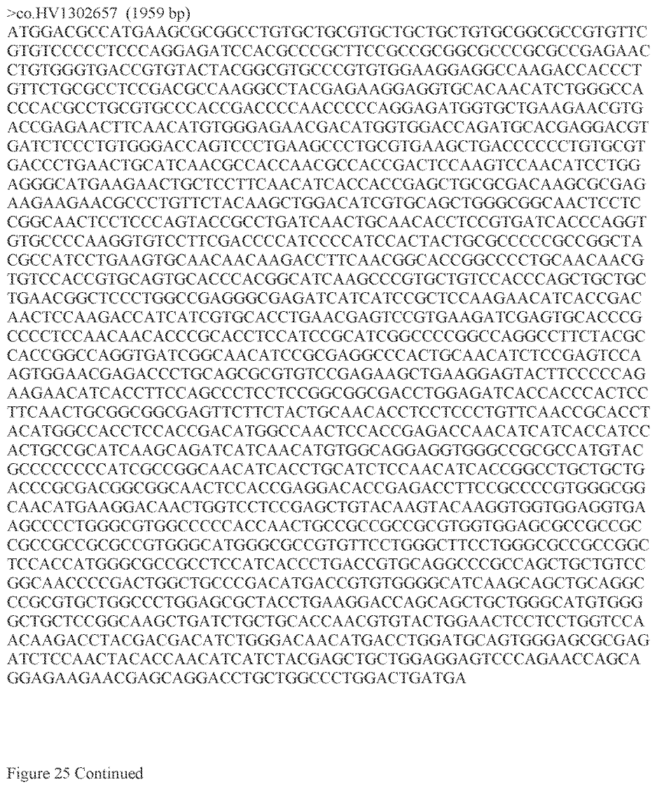

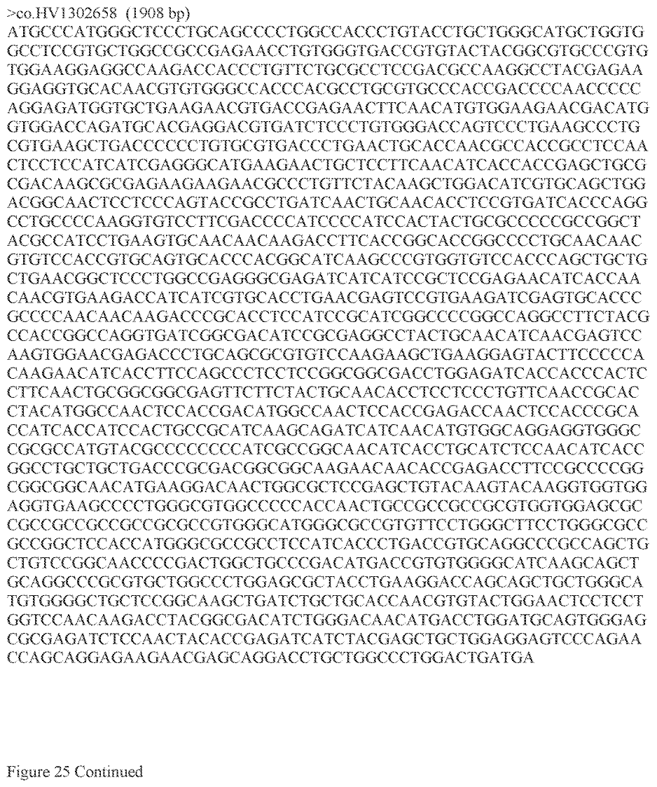

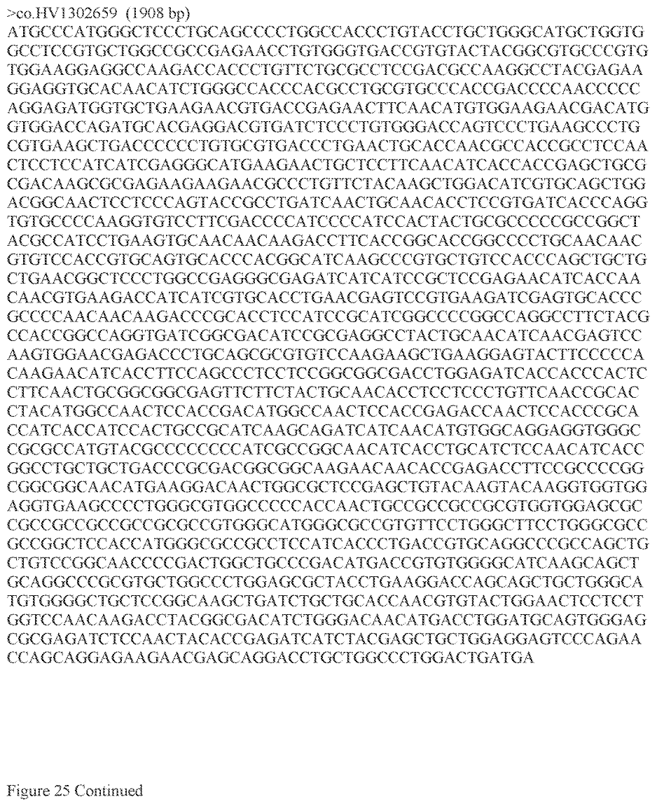

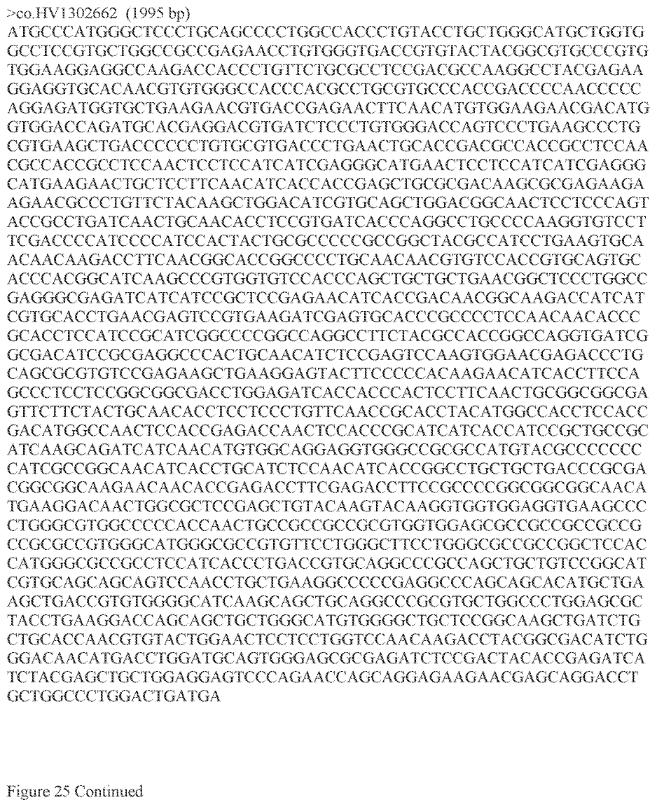

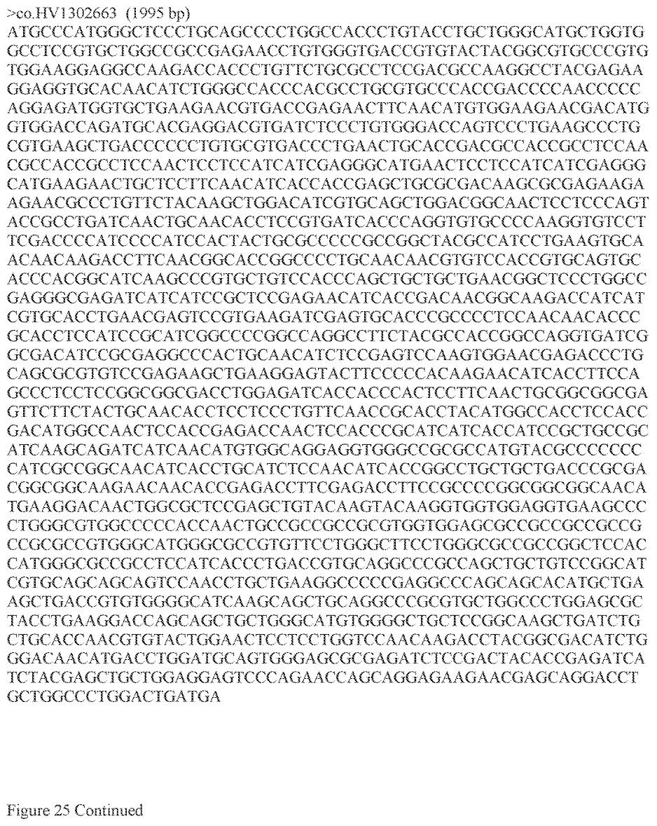

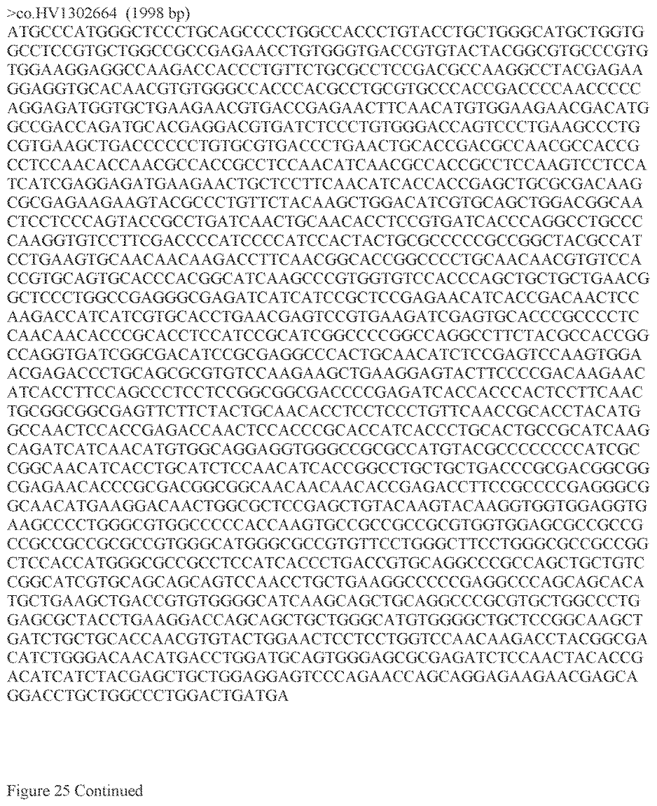

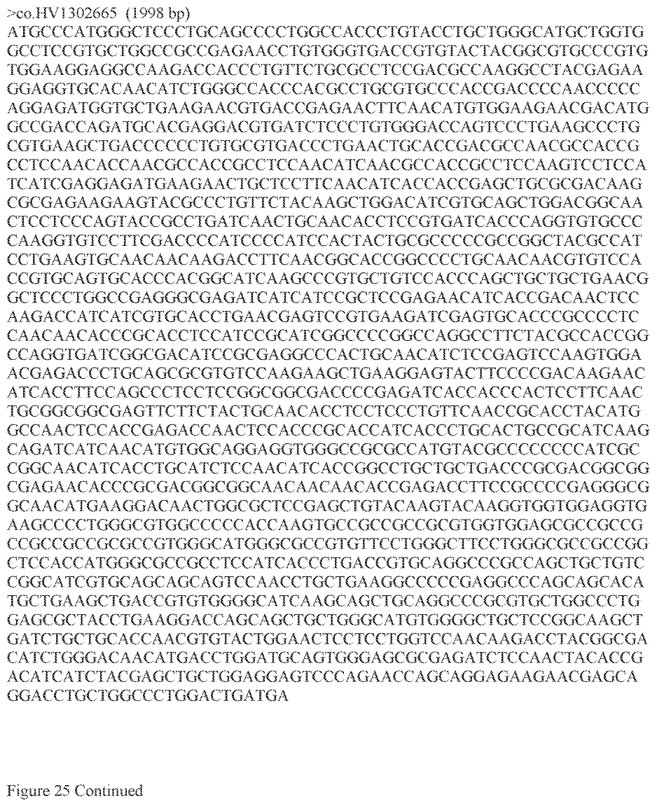

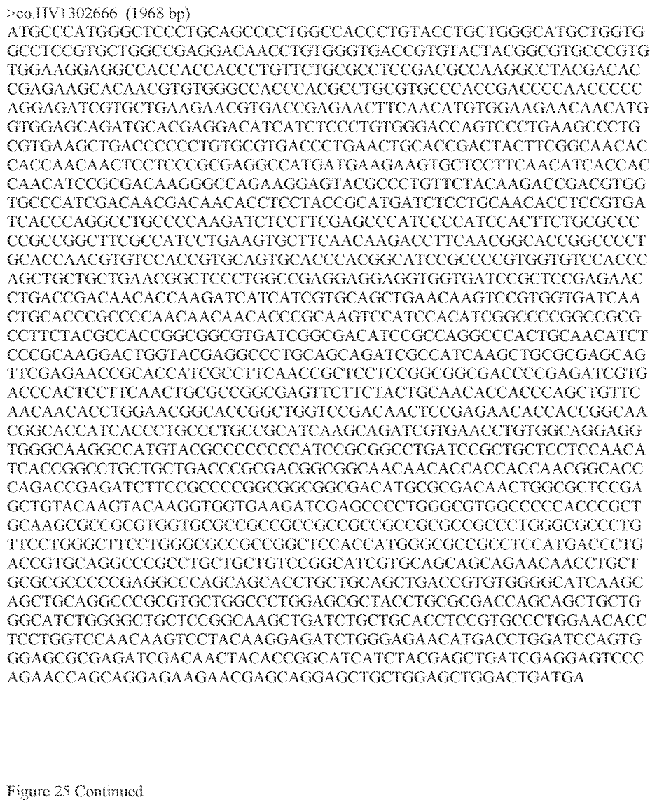

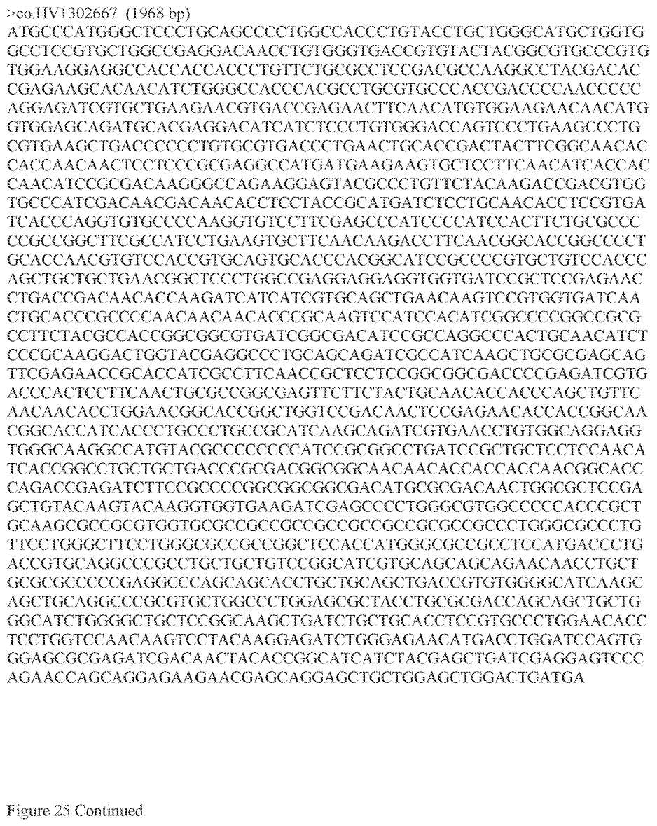

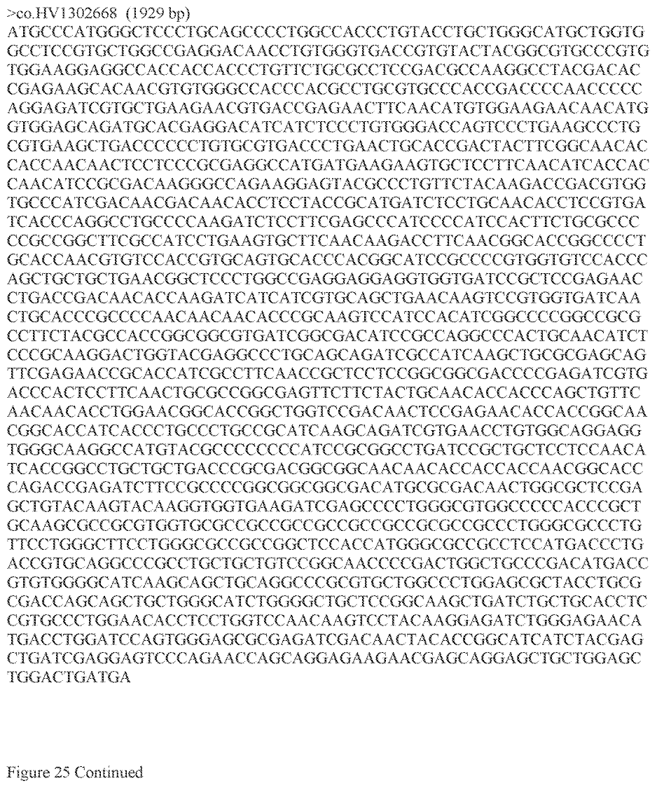

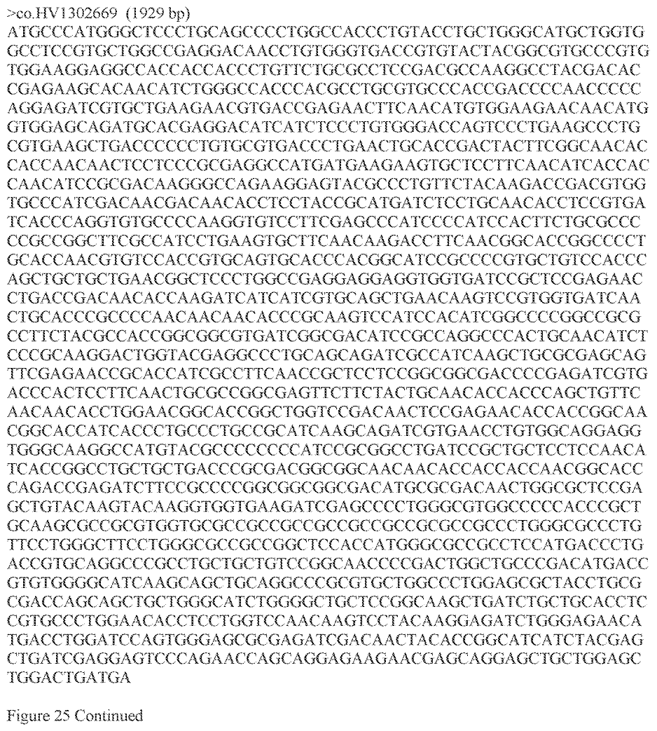

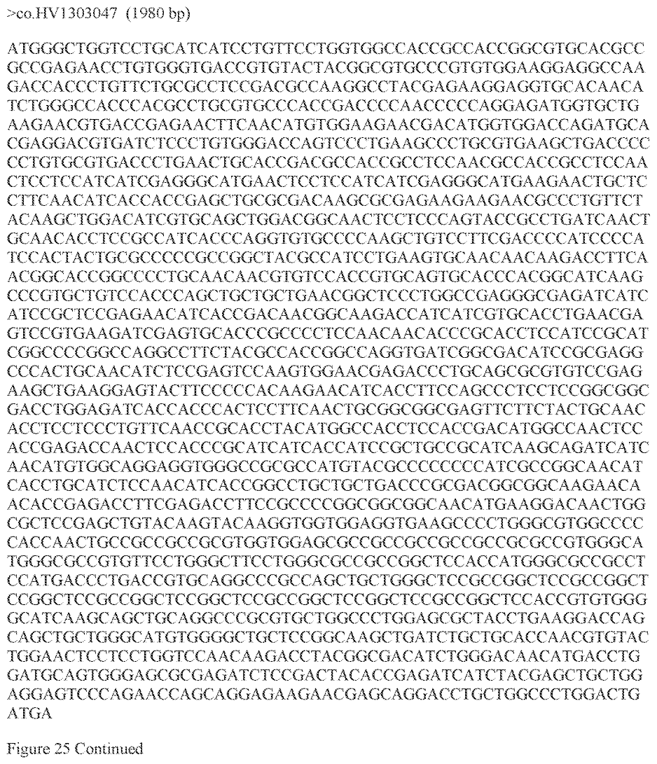

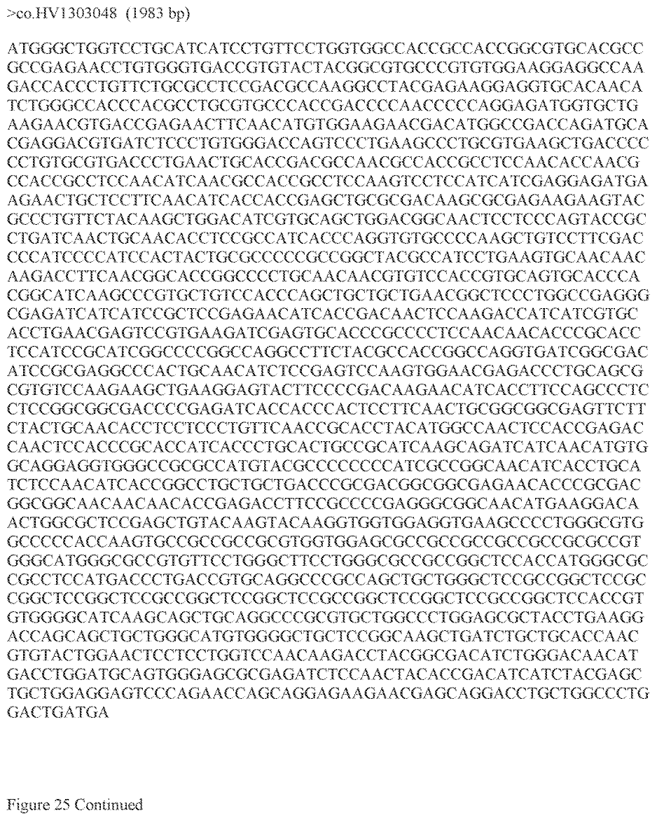

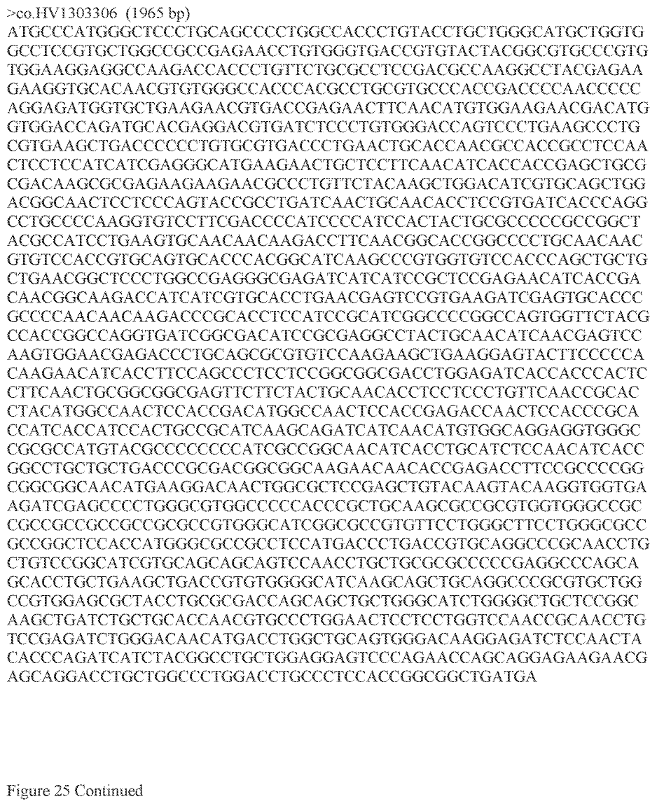

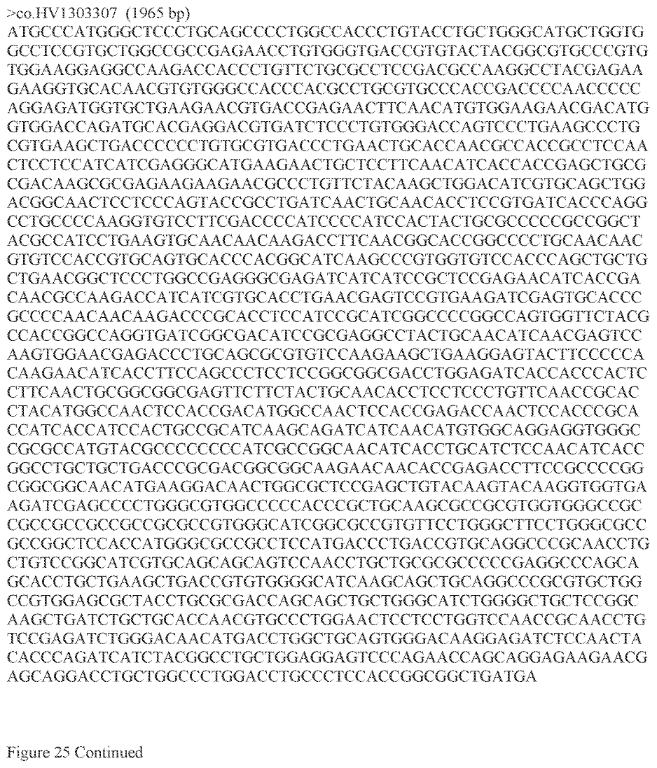

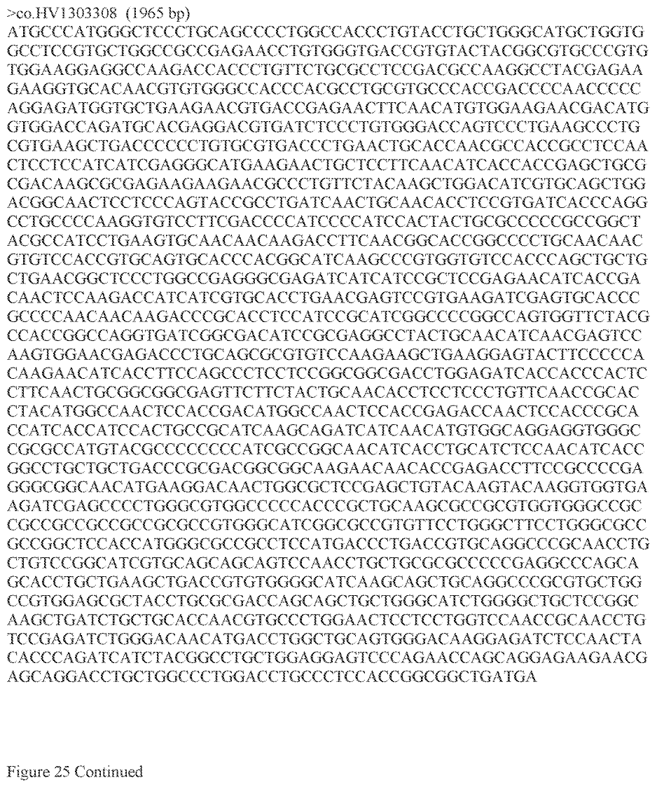

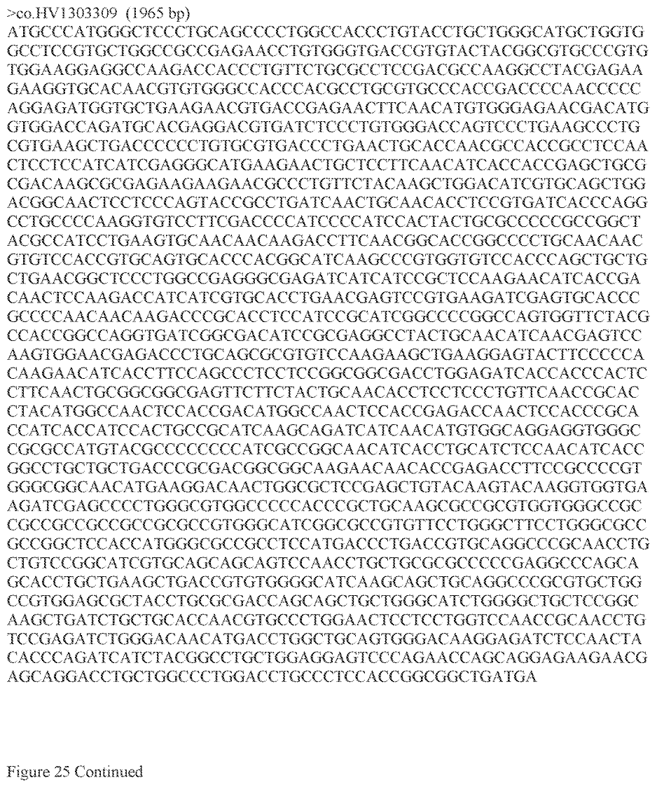

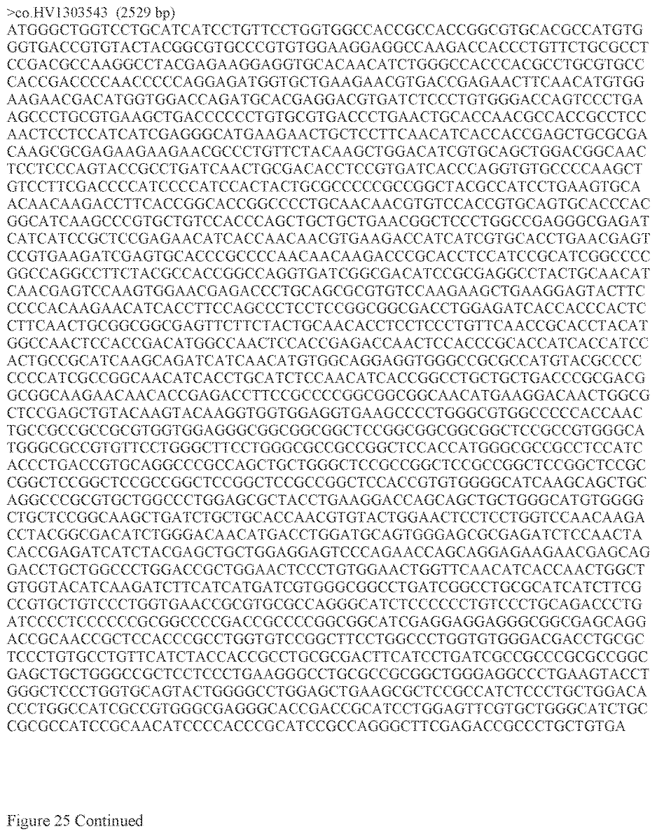

mRNA sequences provided herein, e.g. see FIG. 25 or nucleic acid sequences e.g. see FIG. 21A, 23 or 24A that can be provided as mRNAs, can further comprise a poly A tail (see e.g., 3′UTR with poly A tail in Table 5). In some embodiments, the poly A tail has a length of 101 nucleotides. However, it should be understood that mRNA sequences can comprise different lengths of poly A tail. For example, in some embodiments the poly A tail is about 85 to about 200 nucleotides long. For example, in some embodiments the poly A tail is 85 to 200 nucleotides long. In some embodiments the poly A tail is about 85 to about 110 nucleotides long. In some embodiments the poly A tail is 85 to 110 nucleotides long. In some embodiments the poly A tail is about 90 to about 110 nucleotides long. In some embodiments the poly A tail is 90 to 110 nucleotides long. In RNA sequences, thymine will be uridine. In some embodiments, uridine will be 1-methyl-pseudouridine. In some embodiments, nucleic acids of the invention, including RNA sequences or mRNA sequences, can further comprise any type of modified nucleotides, including, but not limited to 5-methyl-cytidine, 6-methyl-adenosine, or modified uridine.

In certain aspects the invention provides nucleic acids encoding the inventive protein designs. In non-limiting embodiments, the nucleic acids are mRNA, modified or unmodified, suitable for use any use, e.g. but not limited to use as pharmaceutical compositions. In certain embodiments, the nucleic acids are formulated in lipid, such as but not limited to LNPs.

In some embodiments the antibodies are administered as nucleic acids, including but not limited to mRNAs which could be modified and/or unmodified. See US Pub 20180028645A1, US Pub 20170369532, US Pub 20090286852, US Pub 20130111615, US Pub 20130197068, US Pub 20130261172, US Pub 20150038558, US Pub 20160032316, US Pub 20170043037, US Pub 20170327842, US Pub 20180344838A1 at least at paragraphs [0260]-[0281] for non-limiting embodiments of chemical modifications, wherein each content is incorporated by reference in its entirety. In non-limiting embodiments, a modified mRNA comprises pseudouridine. In some embodiments, the modified mRNA comprises 1-methyl-pseudouridine.

mRNAs delivered in LNP formulations have advantages over non-LNPs formulations. See US Pub 20180028645A1.

In certain embodiments the nucleic acid encoding a protein is operably linked to a promoter inserted an expression vector. In certain aspects the compositions comprise a suitable carrier. In certain aspects the compositions comprise a suitable adjuvant.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In one embodiment, the nucleic acid is an RNA molecule. In one embodiment, the RNA molecule is transcribed from a DNA sequence described herein. In some embodiments, the RNA molecule is encoded by one of the inventive sequences. In another embodiment, the nucleotide sequence comprises an RNA sequence transcribed by a DNA sequence encoding the polypeptide sequences described herein, or a variant thereof or a fragment thereof. Accordingly, in one embodiment, the invention provides an RNA molecule encoding one or more of inventive antibodies. The RNA may be plus-stranded. Accordingly, in some embodiments, the RNA molecule can be translated by cells without needing any intervening replication steps such as reverse transcription.

In some embodiments, a RNA molecule of the invention may have a 5′ cap (e.g. but not limited to a 7-methylguanosine, 7mG(5′)ppp(5′)NlmpNp). This cap can enhance in vivo translation of the RNA. The 5′ nucleotide of an RNA molecule useful with the invention may have a 5′ triphosphate group. In a capped RNA this may be linked to a 7-methylguanosine via a 5′-to-5′ bridge. An RNA molecule may have a 3′ poly-A tail. It may also include a poly-A polymerase recognition sequence (e.g. AAUAAA) near its 3′ end. In some embodiments, an RNA molecule useful with the invention may be single-stranded. In some embodiments, an RNA molecule useful with the invention may comprise synthetic RNA.

The recombinant nucleic acid sequence can be an optimized nucleic acid sequence. Such optimization can increase or alter the immunogenicity of the protein. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

Methods for in vitro transfection of mRNA and detection of protein expression are known in the art. Methods for expression and immunogenicity determination of nucleic acid encoded proteins are known in the art.

In some embodiments, the envelope designs can be membrane anchored, for example, for embodiments where the envelope is expressed as a virus like particle.

In some embodiments, a protomer of a disclosed envelope protein can be linked to a ferritin subunit to construct a ferritin nanoparticle. Ferritin nanoparticles and their use for immunization purposes (e.g., for immunization against influenza antigens) have been disclosed in the art (see, e.g., Kanekiyo et al., Nature, 499:102-106, 2013, incorporated by reference herein in its entirety). Ferritin is a globular protein that is found in all animals, bacteria, and plants, and which acts primarily to control the rate and location of polynuclear Fe(III)₂O₃ formation through the transportation of hydrated iron ions and protons to and from a mineralized core. The globular form of the ferritin nanoparticle is made up of monomeric subunits, which are polypeptides having a molecule weight of approximately 17-20 kDa.

Following production, these monomeric subunit proteins self-assemble into the globular ferritin protein. Thus, the globular form of ferritin comprises 24 monomeric, subunit proteins, and has a capsid-like structure having 432 symmetry. Methods of constructing ferritin nanoparticles are known to the person of ordinary skill in the art and are further described herein (see, e.g., Zhang, Int. J. Mol. Sci., 12:5406-5421, 2011, which is incorporated herein by reference in its entirety).

In non-specific examples, the ferritin polypeptide is E. coli ferritin, Helicobacter pylori ferritin, insect ferritin, human light chain ferritin, bullfrog ferritin or a hybrid thereof, such as E. coli-human hybrid ferritin, E. coli-bullfrog hybrid ferritin, or human-bullfrog hybrid ferritin. Exemplary amino acid sequences of ferritin polypeptides and nucleic acid sequences encoding ferritin polypeptides for use to make a ferritin nanoparticle including a recombinant HIV-1 envelope protein can be found in GENBANK, for example at accession numbers ZP_03085328, ZP_06990637, EJB64322.1, AAA35832, NP_000137, AAA49532, AAA49525, AAA49524 and AAA49523, which are specifically incorporated by reference herein in their entirety. In some embodiments, a recombinant protein of the invention can be linked to a ferritin subunit to form a nanoparticle.

Polynucleotides encoding a protomer of any of the disclosed recombinant proteins are also provided. These polynucleotides include DNA, cDNA and RNA sequences which encode the protomer, as well as vectors including the DNA, cDNA and RNA sequences, such as a DNA or RNA vector used for immunization. The genetic code to construct a variety of functionally equivalent nucleic acids, such as nucleic acids which differ in sequence but which encode the same protein sequence, or encode a conjugate or fusion protein including the nucleic acid sequence is known in the art.

Exemplary nucleic acids can be prepared by molecular and cloning techniques. A wide variety of cloning methods, host cells, and in vitro amplification methodologies are well known to persons of skill, and can be used to make the nucleic acids and proteins of the invention.

The polynucleotides encoding a disclosed recombinant envelope can include a recombinant DNA which is incorporated into a vector (such as an expression vector) into an autonomously replicating plasmid or virus or into the genomic DNA of a prokaryote or eukaryote, or which exists as a separate molecule (such as a cDNA) independent of other sequences. The nucleotides can be ribonucleotides, deoxyribonucleotides, or modified forms of nucleotides. The term includes single and double stranded forms of DNA.

Polynucleotide sequences encoding a disclosed envelope can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA and stop codons.

DNA sequences encoding the disclosed recombinant protomer can be expressed in vitro by DNA transfer into a suitable host cell. The cell may be prokaryotic or eukaryotic. The term also includes any progeny of the subject host cell. All progeny may not be identical to the parental cell since there may be mutations that occur during replication. Methods of stable transfer, meaning that the foreign DNA is continuously maintained in the host, are known in the art.

Host systems for recombinant production can include microbial, yeast, insect and mammalian organisms. Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, e.g., Helgason and Miller (Eds.), 2012, Basic Cell Culture Protocols (Methods in Molecular Biology), 4.sup.th Ed., Humana Press). Examples of mammalian host cell lines are VERO and HeLa cells, CHO cells, and WI38, BHK, and COS cell lines, although cell lines may be used, such as cells designed to provide higher expression, desirable glycosylation patterns, or other features. In some embodiments, the host cells include HEK293 cells or derivatives thereof, such as GnTI^{-/-} cells, or HEK-293F cells.

A nucleic acid molecule encoding a protomer can be included in a formulation, for example an LNP formulation, for expression of the immunogen in a host cell, or for immunization of a subject as disclosed herein. A nucleic acid molecule encoding a protomer can be included in a viral vector, for example, for expression of the immunogen in a host cell, or for immunization of a subject as disclosed herein. In some embodiments, the viral vectors are administered to a subject as part of a prime-boost vaccination. In several embodiments, the viral vectors are included in a vaccine, such as a prime vaccine or a booster vaccine for use in a prime-boost vaccination.

In several examples, the viral vector can be replication-competent. For example, the viral vector can have a mutation in the viral genome that does not inhibit viral replication in host cells. The viral vector also can be conditionally replication-competent. In other examples, the viral vector is replication-deficient in host cells.

A number of viral vectors have been constructed, that can be used to express the disclosed antigens, including polyoma, i.e., SV40 (Madzak et al., 1992, J. Gen. Virol., 73:15331536), adenovirus (Berkner, 1992, Cur. Top. Microbiol. Immunol., 158:39-6; Berliner et al., 1988, Bio Techniques, 6:616-629; Gorziglia et al., 1992, J. Virol., 66:4407-4412; Quantin et al., 1992, Proc. Natl. Acad. Sci. USA, 89:2581-2584; Rosenfeld et al., 1992, Cell, 68:143-155; Wilkinson et al., 1992, Nucl. Acids Res., 20:2233-2239; Stratford-Perricaudet et al., 1990, Hum. Gene Ther., 1:241-256), vaccinia virus (Mackett et al., 1992, Biotechnology, 24:495-499), adeno-associated virus (Muzyczka, 1992, Curr. Top. Microbiol. Immunol., 158:91-123; On et al., 1990, Gene, 89:279-282), herpes viruses including HSV and EBV (Margolskee, 1992, Curr. Top. Microbiol. Immunol., 158:67-90; Johnson et al., 1992, J. Virol., 66:29522965; Fink et al., 1992, Hum. Gene Ther. 3:11-19; Breakfield et al., 1987, Mol. Neurobiol., 1:337-371; Fresse et al., 1990, Biochem. Pharmacol., 40:2189-2199), Sindbis viruses (H. Herweijer et al., 1995, Human Gene Therapy 6:1161-1167; U.S. Pat. Nos. 5,091,309 and 5,2217,879), alphaviruses (S. Schlesinger, 1993, Trends Biotechnol. 11:18-22; I. Frolov et al., 1996, Proc. Natl. Acad. Sci. USA 93:11371-11377) and retroviruses of avian (Brandyopadhyay et al., 1984, Mol. Cell Biol., 4:749-754; Petropouplos et al., 1992, J. Virol., 66:3391-3397), murine (Miller, 1992, Curr. Top. Microbiol. Immunol., 158:1-24; Miller et al., 1985, Mol. Cell Biol., 5:431-437; Sorge et al., 1984, Mol. Cell Biol., 4:1730-1737; Mann et al., 1985, J. Virol., 54:401-407), and human origin (Page et al., 1990, J. Virol., 64:5370-5276; Buchschalcher et al., 1992, J. Virol., 66:2731-2739). Baculovirus (Autographa californica multinuclear polyhedrosis virus; AcMNPV) vectors are also known in the art and may be obtained from commercial sources (such as PharMingen, San Diego, Calif.; Protein Sciences Corp., Meriden, Conn.; Stratagene, La Jolla, Calif.).

In several embodiments, the viral vector can include an adenoviral vector that expresses a protomer of the invention. Adenovirus from various origins, subtypes, or mixture of subtypes can be used as the source of the viral genome for the adenoviral vector. Non-human adenovirus (e.g., simian, chimpanzee, gorilla, avian, canine, ovine, or bovine adenoviruses) can be used to generate the adenoviral vector. For example, a simian adenovirus can be used as the source of the viral genome of the adenoviral vector. A simian adenovirus can be of serotype 1, 3, 7, 11, 16, 18, 19, 20, 27, 33, 38, 39, 48, 49, 50, or any other simian adenoviral serotype. A simian adenovirus can be referred to by using any suitable abbreviation known in the art, such as, for example, SV, SAdV, SAV or sAV. In some examples, a simian adenoviral vector is a simian adenoviral vector of serotype 3, 7, 11, 16, 18, 19, 20, 27, 33, 38, or 39. In one example, a chimpanzee serotype C Ad3 vector is used (see, e.g., Peruzzi et al., Vaccine, 27:1293-1300, 2009). Human adenovirus can be used as the source of the viral genome for the adenoviral vector. Human adenovirus can be of various subgroups or serotypes. For instance, an adenovirus can be of subgroup A (e.g., serotypes 12, 18, and 31), subgroup B (e.g., serotypes 3, 7, 11, 14, 16, 21, 34, 35, and 50), subgroup C (e.g., serotypes 1, 2, 5, and 6), subgroup D (e.g., serotypes 8, 9, 10, 13, 15, 17, 19, 20, 22, 23, 24, 25, 26, 27, 28, 29, 30, 32, 33, 36-39, and 42-48), subgroup E (e.g., serotype 4), subgroup F (e.g., serotypes 40 and 41), an unclassified serogroup (e.g., serotypes 49 and 51), or any other adenoviral serotype. The person of ordinary skill in the art is familiar with replication competent and deficient adenoviral vectors (including singly and multiply replication deficient adenoviral vectors). Examples of replication-deficient adenoviral vectors, including multiply replication-deficient adenoviral vectors, are disclosed in U.S. Pat. Nos. 5,837,511; 5,851,806; 5,994,106; 6,127,175; 6,482,616; and 7,195,896, and International Patent Publication Nos. WO 94/28152, WO 95/02697, WO 95/16772, WO 95/34671, WO 96/22378, WO 97/12986, WO 97/21826, and WO 03/022311.

In some embodiments, a virus-like particle (VLP) is provided that comprises a recombinant protomer of the invention. In some embodiments, a virus-like particle (VLP) is provided that includes a recombinant trimer of the invention. Such VLPs can include an envelope trimer that is membrane anchored by a C-terminal transmembrane domain. VLPs lack the viral components that are required for virus replication and thus represent a highly attenuated, replication-incompetent form of a virus. However, the VLP can display a polypeptide that is analogous to that expressed on infectious virus particles and can eliciting an immune response to the corresponding envelope when administered to a subject. Virus like particles and methods of their production are known and familiar to the person of ordinary skill in the art, and viral proteins from several viruses are known to form VLPs, including human papillomavirus, HIV (Kang et al., Biol. Chem. 380: 353-64 (1999)), Semliki-Forest virus (Notka et al., Biol. Chem. 380: 341-52 (1999)), human polyomavirus (Goldmann et al., J. Virol. 73: 4465-9 (1999)), rotavirus (Jiang et al., Vaccine 17: 1005-13 (1999)), parvovirus (Casal, Biotechnology and Applied Biochemistry, Vol 29, Part 2, pp 141-150 (1999)), canine parvovirus (Hurtado et al., J. Virol. 70: 5422-9 (1996)), hepatitis E virus (Li et al., J. Virol. 71: 7207-13 (1997)), and Newcastle disease virus. The formation of such VLPs can be detected by any suitable technique. Examples of suitable techniques known in the art for detection of VLPs in a medium include, e.g., electron microscopy techniques, dynamic light scattering (DLS), selective chromatographic separation (e.g., ion exchange, hydrophobic interaction, and/or size exclusion chromatographic separation of the VLPs) and density gradient centrifugation.

The immunogens of the invention could be combined with any suitable adjuvant.

In certain aspects the invention contemplates using immunogenic compositions wherein immunogens are delivered as recombinant proteins. Various methods for production and purification of recombinant proteins, including trimers such as but not limited to SOSIP based trimers, suitable for use in immunization are known in the art. In certain embodiments recombinant proteins are produced in CHO cells.

The immunogenic envelopes can also be administered as a protein boost in combination with a variety of nucleic acid envelope primes (e.g., HIV-1 Envs delivered as DNA expressed in viral or bacterial vectors).

Dosing of proteins and nucleic acids can be readily determined by a skilled artisan. A single dose of nucleic acid can range from a few nanograms (ng) to a few micrograms (g) or milligram of a single immunogenic nucleic acid. Recombinant protein dose can range from a few g micrograms to a few hundred micrograms, or milligrams of a single immunogenic polypeptide.

Administration: The compositions can be formulated with appropriate carriers using known techniques to yield compositions suitable for various routes of administration. In certain embodiments the compositions are delivered via intramuscular (IM), via subcutaneous, via intravenous, via nasal, via mucosal routes, or any other suitable route of immunization.

The compositions can be formulated with appropriate carriers and adjuvants using techniques to yield compositions suitable for immunization. The compositions can include an adjuvant, such as, for example but not limited to, alum, 3M052, formulated in alum or SE, poly IC, MF-59 or other squalene-based adjuvant, ASOIB, or other liposomal based adjuvant suitable for protein or nucleic acid immunization. In certain embodiments, the adjuvant is GSK AS01E adjuvant containing MPL and QS21. This adjuvant has been shown by GSK to be as potent as the similar adjuvant AS01B but to be less reactogenic using HBsAg as vaccine antigen (Leroux-Roels et al., IABS Conference, April 2013). In certain embodiments, TLR agonists are used as adjuvants. In other embodiment, adjuvants which break immune tolerance are included in the immunogenic compositions.

In certain embodiments, the compositions and methods comprise any suitable agent or immune modulation which could modulate mechanisms of host immune tolerance and release of the induced antibodies. In non-limiting embodiments modulation includes PD-1 blockade; T regulatory cell depletion; CD40L hyperstimulation; soluble antigen administration, wherein the soluble antigen is designed such that the soluble agent eliminates B cells targeting dominant epitopes, or a combination thereof. In certain embodiments, an immunomodulatory agent is administered in at time and in an amount sufficient for transient modulation of the subject's immune response so as to induce an immune response which comprises broad neutralizing antibodies against HIV-1 envelope. Non-limiting examples of such agents is any one of the agents described herein: e.g. chloroquine (CQ), PTP1B Inhibitor—CAS 765317-72-4—Calbiochem or MSI 1436 clodronate or any other bisphosphonate; a Foxo1 inhibitor, e.g. 344355|Foxo1 Inhibitor, AS1842856—Calbiochem; Gleevac, anti-CD25 antibody, anti-CCR4 Ab, an agent which binds to a B cell receptor for a dominant HIV-1 envelope epitope, or any combination thereof. In non-limiting embodiments, the modulation includes administering an anti-CTLA4 antibody. Non-limiting examples are ipilimumab and tremelimumab. In certain embodiments, the methods comprise administering a second immunomodulatory agent, wherein the second and first immunomodulatory agents are different.

There are various host mechanisms that control bNAbs. For example, highly somatically mutated antibodies become autoreactive and/or less fit (Immunity 8: 751, 1998; PloS Comp. Biol. 6 e1000800, 2010; J. Thoret. Biol. 164:37, 1993); Polyreactive/autoreactive naïve B cell receptors (unmutated common ancestors of clonal lineages) can lead to deletion of Ab precursors (Nature 373: 252, 1995; PNAS 107: 181, 2010; J. Immunol. 187: 3785, 2011); Abs with long HCDR3 can be limited by tolerance deletion (JI 162: 6060, 1999; JCI 108: 879, 2001). BnAb knock-in mouse models are providing insights into the various mechanisms of tolerance control of MPER bnAb induction (deletion, anergy, receptor editing). Other variations of tolerance control likely will be operative in limiting bnAbs with long HCDR3s, high levels of somatic hypermutations.

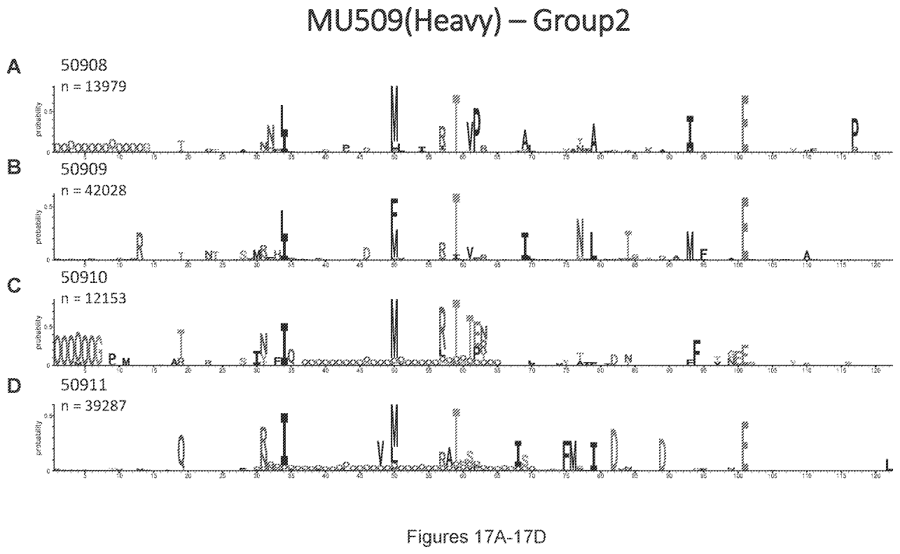

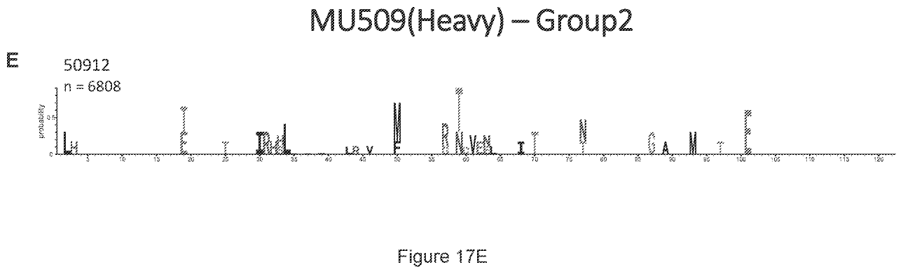

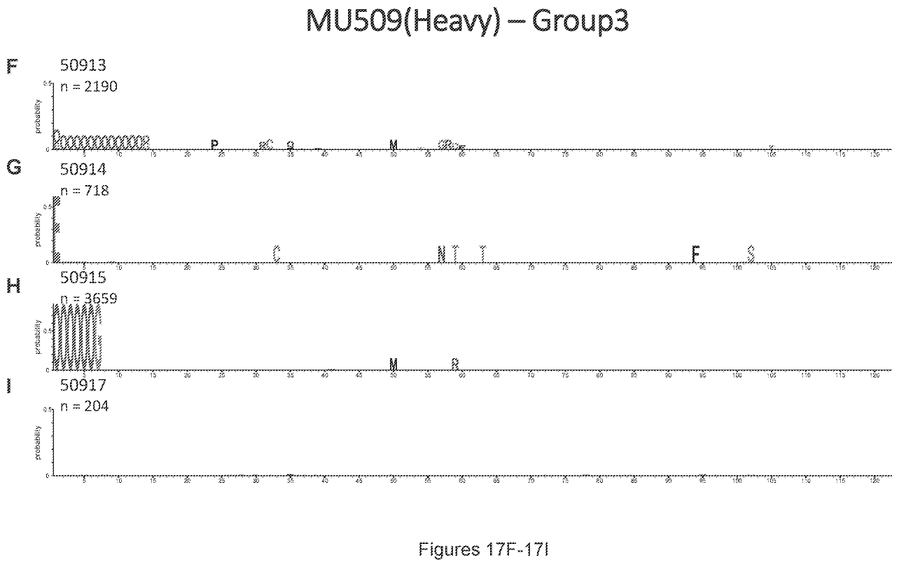

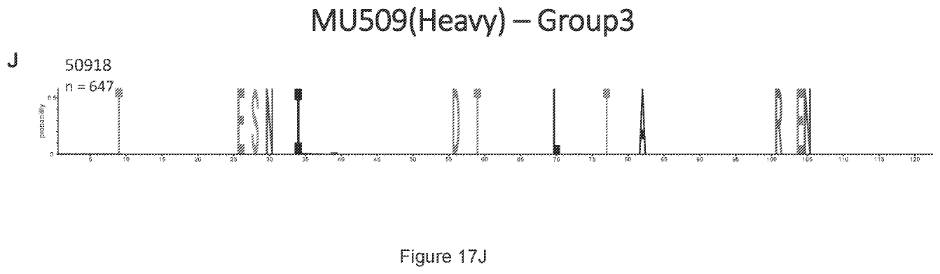

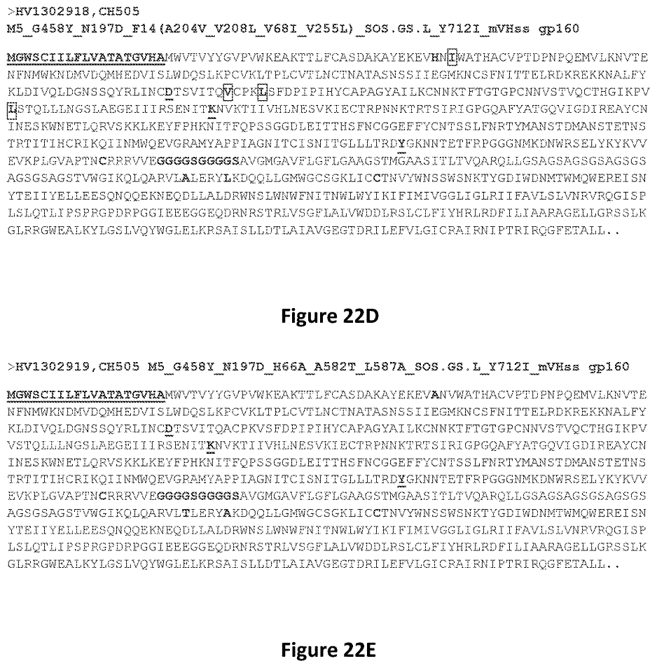

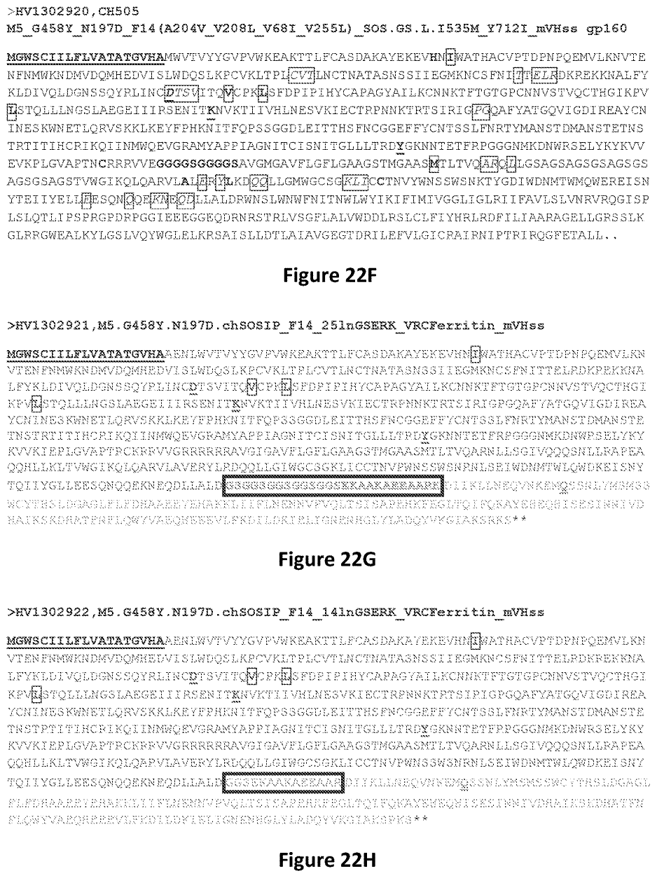

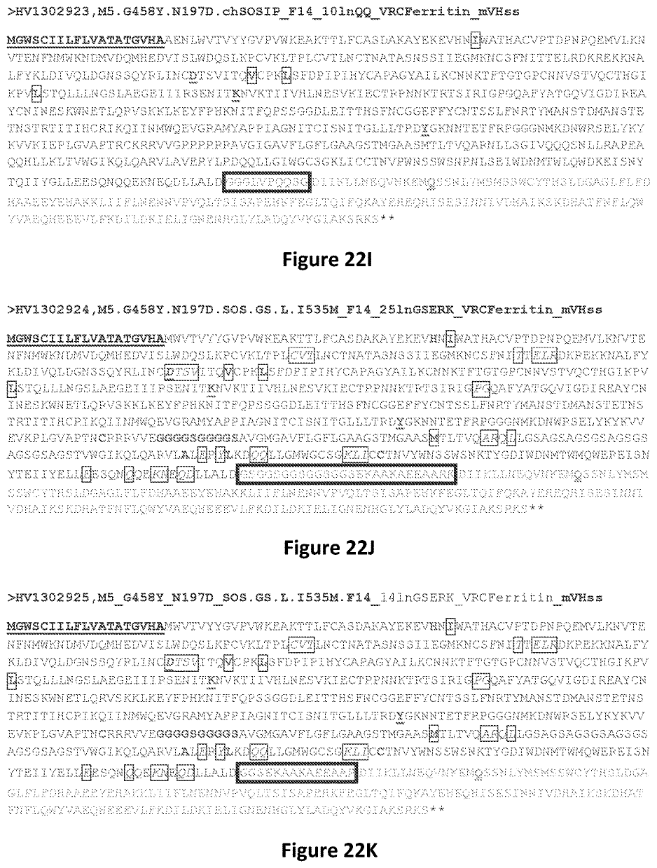

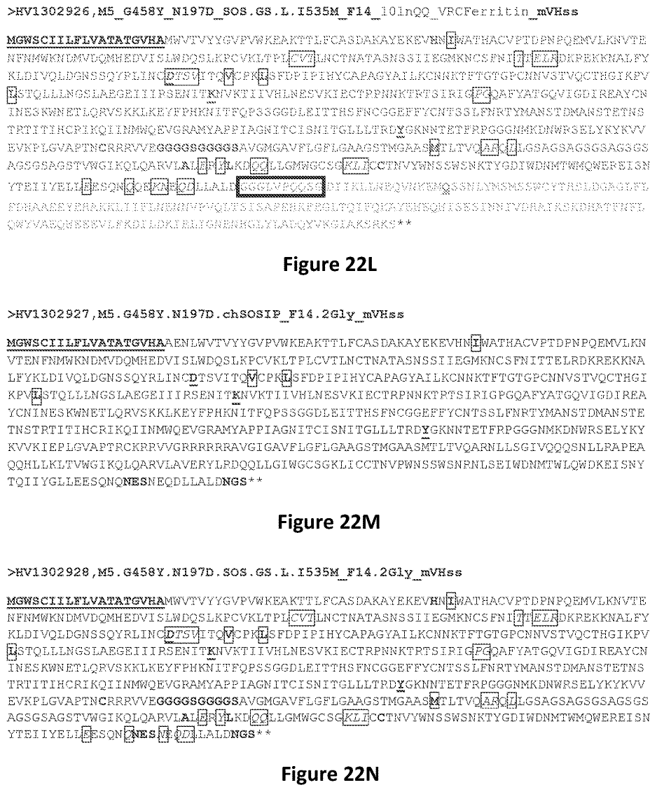

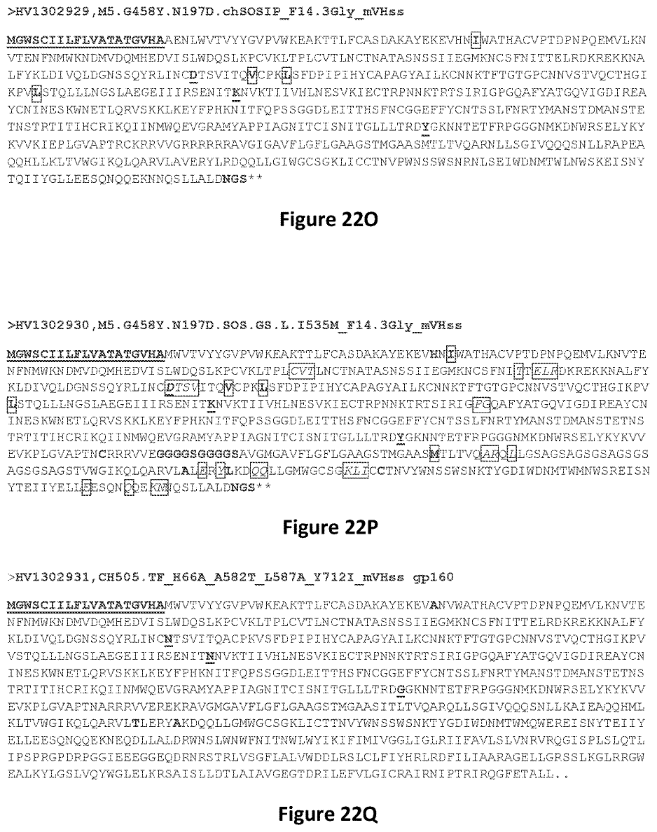

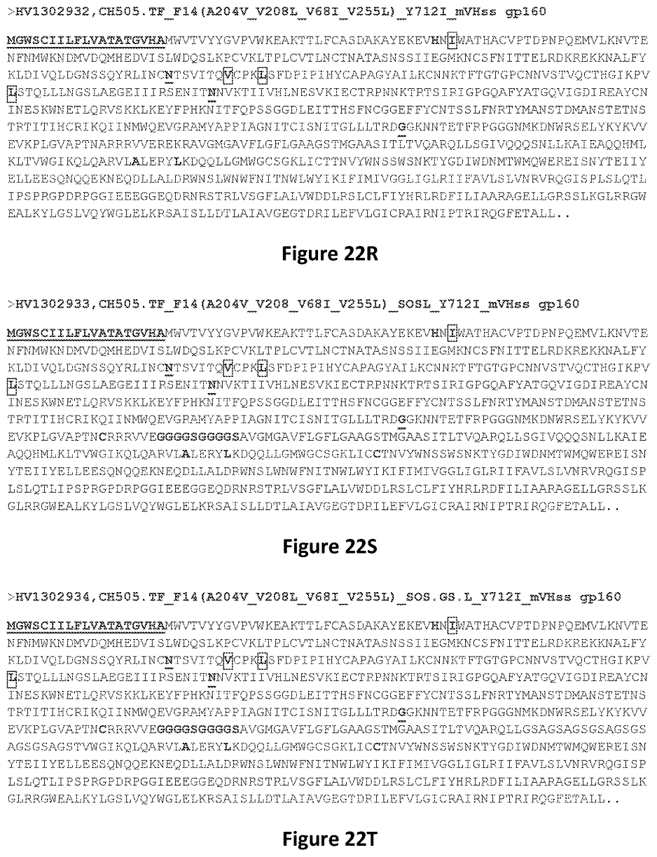

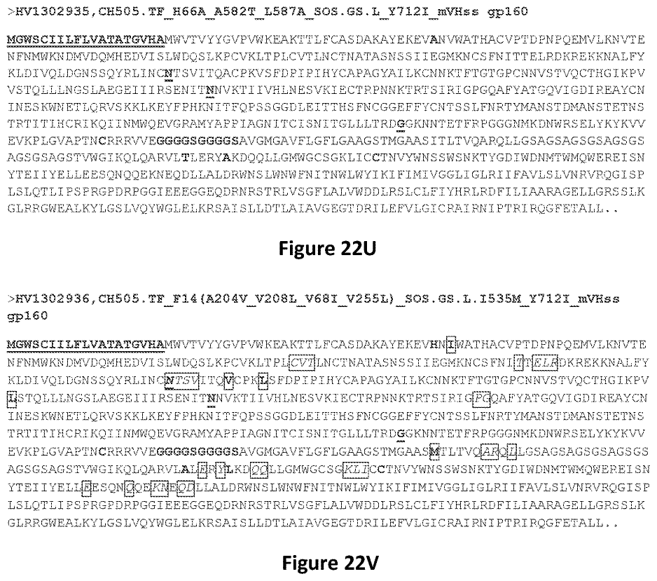

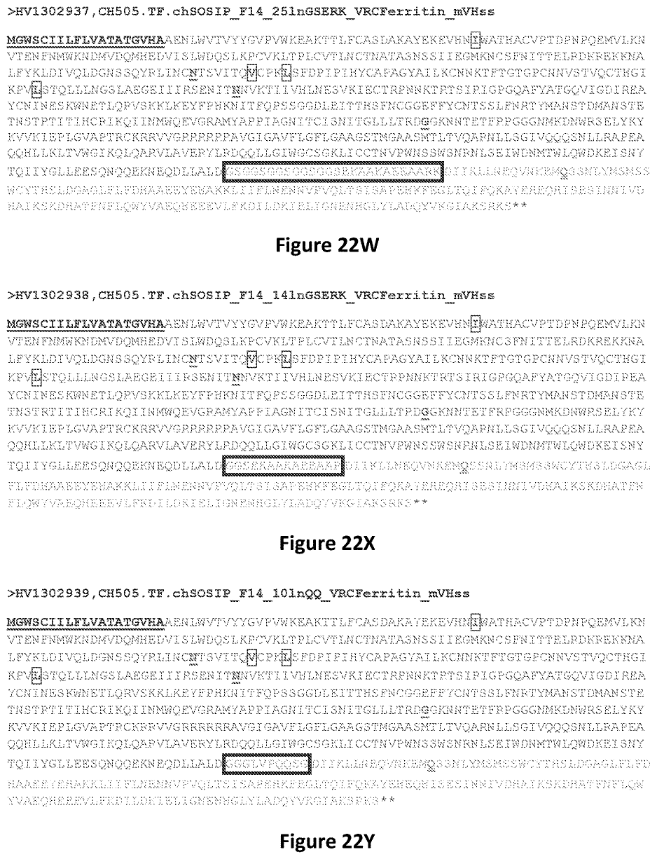

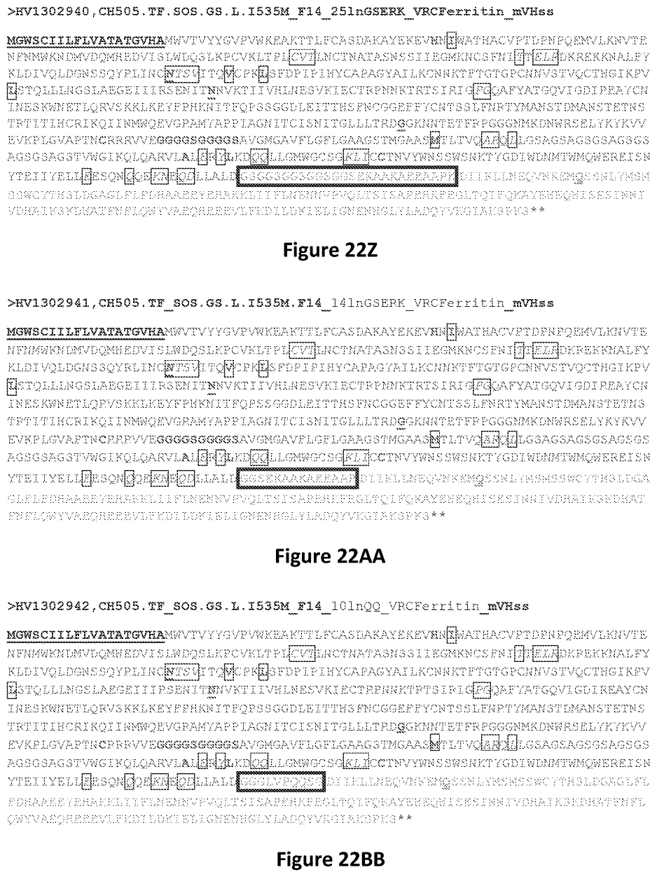

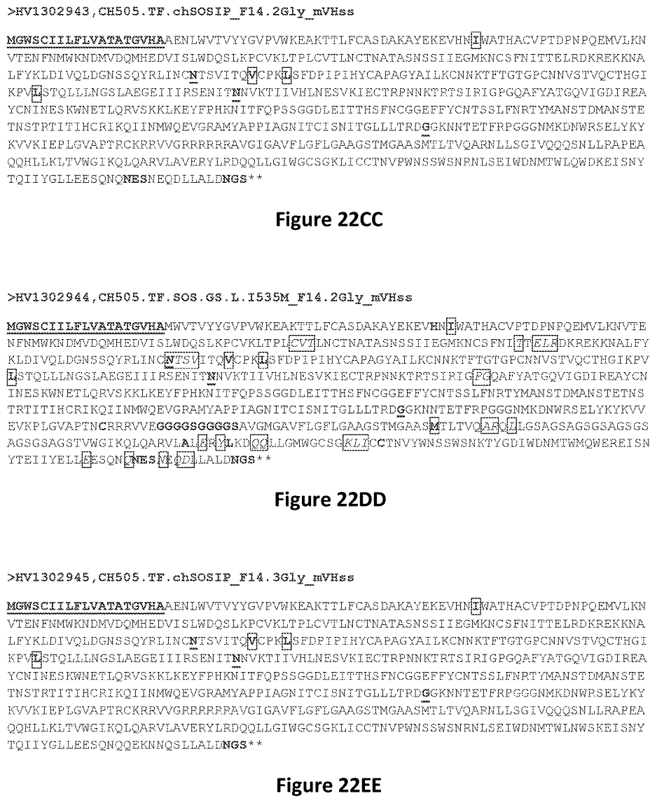

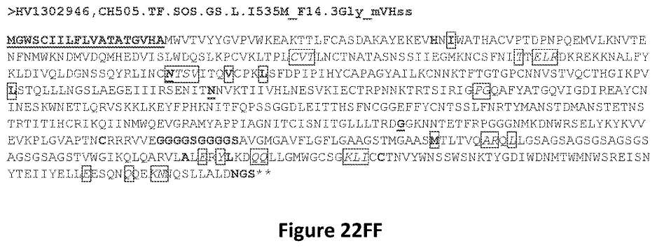

For a summary of CH505 sequences and designs see WO2017151801, e.g. but not limited to Table 1, FIGS. 22-24 in WO2017151801, and FIG. 17 in WO2014042669.

It is readily understood that the envelope glycoproteins referenced in various examples and figures comprise a signal/leader sequence. It is well known in the art that HIV-1 envelope glycoprotein is a secretory protein with a signal or leader peptide sequence that is removed during processing and recombinant expression (without removal of the signal peptide, the protein is not secreted). See for example Li et al. Control of expression, glycosylation, and secretion of HIV-1 gp120 by homologous and heterologous signal sequences. Virology 204(1):266-78 (1994) (“Li et al. 1994”), at first paragraph, and Li et al. Effects of inefficient cleavage of the signal sequence of HIV-1 gp120 on its association with calnexin, folding, and intracellular transport. PNAS 93:9606-9611 (1996) (“Li et al. 1996”), at 9609. Any suitable signal sequence could be used. In some embodiments the leader sequence is the endogenous leader sequence. Most of the gp120 and gp160 amino acid sequences include the endogenous leader sequence. In other non-limiting examples, the leader sequence is human Tissue Plasminogen Activator (TPA) sequence, human CD5 leader sequence (e.g. MPMGSLQPLATLYLLGMLVASVLA). Most of the chimeric designs include CD5 leader sequence. A skilled artisan appreciates that when used as immunogens, and for example when recombinantly produced, the amino acid sequences of these proteins do not comprise the leader peptide sequences. Thus, in some embodiments, the polypeptides of the invention comprise the amino acids of the HIV-1 envelope immediately following the signal peptide.

### HIV-1 Envelope Trimers and Other Envelope Designs

Stabilized HIV-1 Env trimer immunogens show enhanced antigenicity for broadly neutralizing antibodies and are not recognized by non-neutralizing antibodies. In some embodiments the envelopes, including but not limited to trimers are further multimerized, and/or used as particulate, high-density array in liposomes or other particles, for example but not limited to nanoparticles. Any one of the envelopes of the invention could be designed and expressed as described herein.

A stabilized chimeric SOSIP design was used to generate CH505 trimers. This design was applicable to diverse viruses from multiple clades.

Elicitation of neutralizing antibodies is one goal for antibody-based vaccines. Neutralizing antibodies target the native trimeric HIV-1 Env on the surface virions. The trimeric HIV-1 envelope protein consists of three protomers each containing a gp120 and gp41 heterodimer. Recent immunogen design efforts have generated soluble near-native mimics of the Env trimer that bind to neutralizing antibodies but not non-neutralizing antibodies. The recapitulation of the native trimer could be a key component of vaccine induction of neutralizing antibodies. Neutralizing Abs target the native trimeric HIV-1 Env on the surface of viruses (Poignard et al. J Virol. 2003 January; 77(1):353-65; Parren et al. J Virol. 1998 December; 72(12):10270-4.; Yang et al. J Virol. 2006 November; 80(22):11404-8.). The HIV-1 Env protein consists of three protomers of gp120 and gp41 heterodimers that are noncovalently linked together (Center et al. J Virol. 2002 August; 76(15):7863-7.). Soluble near-native trimers preferentially bind neutralizing antibodies as opposed to non-neutralizing antibodies (Sanders et al. PLoS Pathog. 2013 September; 9(9): e1003618).

Sequential Env vaccination has elicited broad neutralization in the plasma of one macaque. The overall goal is to increase the frequency of vaccine induction of bNAbs in the plasma of primates with Env vaccination. Without being bound by theory, vaccination with immunogens that target bnAb B cell lineage and mimic native trimers will increase the frequency of broadly neutralizing plasma antibodies. One goal is increasing the frequency of vaccine induction of bnAb in the plasma of primates by Env vaccination. It is expected that vaccination with immunogens that target bnAb B cell lineages and mimic the native trimers on virions will increase the frequency of broadly neutralizing plasma antibodies.

Previous work has shown that CH505 derived soluble trimers are hard to produce. From a study published by Julien et al in 2015 (Proc Natl Acad Sci USA. 2015 Sep. 22; 112(38): 11947-11952) it was shown that while CH505 produced comparable amounts of protein by transient transfection, only 5% of the CH505 protein formed trimer which 5 times lower than the gold standard viral strain BG505. Provided here are non-limiting embodiments of well-folded trimers for Env immunizations.

Near-native soluble trimers using the 6R.SOSIP.664 design are capable of generating autologous tier 2 neutralizing plasma antibodies in the plasma (Sanders et al. 2015), which provides a starting point for designing immunogens to elicit broadly neutralizing antibodies. While these trimers are preferentially antigenic for neutralizing antibodies, they still possess the ability to expose the V3 loop, which generally results in strain-specific binding and neutralizing antibodies after vaccination. Using the unliganded structure the BG505.6R.SOSIP.664 has been stabilized by adding cysteines at position 201 and 433 to constrain the conformational flexibility such that the V3 loop is maintained unexposed (Kwon et al. Nat Struct Mol Biol. 2015 July; 22(7): 522-531.).

Provided are engineered trimeric immunogens derived from multiple viruses from CH505. Chimeric 6R.SOSIP.664, chimeric disulfide stabilized (DS) 6R.SOSIP.664 (Kwon et al Nat Struct Mol Biol. 2015 July; 22(7): 522-531), chimeric 6R.SOSIP.664v4.1 (DeTaeye et al. Cell. 2015 Dec. 17; 163(7):1702-15. Doi: 10.1016/j.cell.2015.11.056), and chimeric 6R.SOSIP.664v4.2 (DeTaeye et al. Cell. 2015 Dec. 17; 163(7):1702-15. Doi: 10.1016/j.cell.2015.11.056) were generated. The 6R.SOSIP.664 is the basis for all of these designs and is made as a chimera of C.CH0505 and A.BG505. The gp120 of C.CH505 was fused with the BG505 inner domain gp120 sequence within the alpha helix 5 (a5) to result in the chimeric protein. The chimeric gp120 is disulfide linked to the A.BG505 gp41 as outlined by Sanders et al. (PLoS Pathog. 2013 September; 9(9): e1003618). These immunogens were designed as chimeric proteins that possess the BG505 gp41 connected to the CH505 gp120, since the BG505 strain is particularly adept at forming well-folded, closed trimers. This envelope design retains the CH505 CD4 binding site that is targeted by the CH103 and CH235 broadly neutralizing antibody lineages that were isolated from CH505.

Based on the various designs, any other suitable envelope, for example but not limited to CH505 envelopes as described in WO2014042669 can be designed.

Recombinant envelopes as trimers could be produced and purified by any suitable method. For a non-limiting example of purification methods see Ringe R P, Yasmeen A, Ozorowski G, Go E P, Pritchard L K, Guttman M, Ketas T A, Cottrell C A, Wilson I A, Sanders R W, Cupo A, Crispin M, Lee K K, Desaire H, Ward A B, Klasse P J, Moore J P. 2015. Influences on the design and purification of soluble, recombinant native-like HIV-1 envelope glycoprotein trimers. J Virol 89:12189-12210. doi:10.1128/JVI.01768-15.

### Multimeric Envelopes

Presentation of antigens as particulates reduces the B cell receptor affinity necessary for signal transduction and expansion (See Baptista et al. EMBO J. 2000 Feb. 15; 19(4): 513-520). Displaying multiple copies of the antigen on a particle provides an avidity effect that can overcome the low affinity between the antigen and B cell receptor. The initial B cell receptor specific for pathogens can be low affinity, which precludes vaccines from being able to stimulate and expand B cells of interest. In particular, very few naïve B cells from which HIV-1 broadly neutralizing antibodies arise can bind to soluble HIV-1 Envelope. Provided are envelopes, including but not limited to trimers as particulate, high-density array on liposomes or other particles, for example but not limited to nanoparticles. See e.g. He et al. Nature Communications 7, Article number: 12041 (2016), doi:10.1038/ncomms12041; Bamrungsap et al. Nanomedicine, 2012, 7 (8), 1253-1271.

To improve the interaction between the naïve B cell receptor and immunogens, envelope designed can be created to wherein the envelope is presented on particles, e.g. but not limited to nanoparticle. In some embodiments, the HIV-1 Envelope trimer could be fused to ferritin. Ferritin protein self assembles into a small nanoparticle with three-fold axis of symmetry. At these axes the envelope protein is fused. Therefore, the assembly of the three-fold axis also clusters three HIV-1 envelope protomers together to form an envelope trimer. Each ferritin particle has 8 axes which equates to 8 trimers being displayed per particle. See e.g. Sliepen et al. Retrovirology 201512:82, DOI: 10.1186/s12977-015-0210-4.

Any suitable ferritin sequence could be used. In non-limiting embodiments, ferritin sequences are disclosed in WO/2018/005558, incorporated by reference in its entirety.

Ferritin nanoparticle linkers: The ability to form HIV-1 envelope ferritin nanoparticles relies self-assembly of 24 ferritin subunits into a single ferritin nanoparticle. The addition of a ferritin subunit to the C-terminus of HIV-1 envelope may interfere with the ability of the ferritin subunit to fold properly and or associate with other ferritin subunits. When expressed alone ferritin readily forms 24-subunit nanoparticles, however appending it to envelope only yields nanoparticles for certain envelopes. Since the ferritin nanoparticle forms in the absence of envelope, the envelope could be sterically hindering the association of ferritin subunits. Thus, ferritin with elongated glycine-serine linkers can be used to further distance the envelope from the ferritin subunit. To make sure that the glycine linker is attached to ferritin at the correct position, constructs that attach at second amino acid position or the fifth amino acid position were crated. The first four N-terminal amino acids of natural Helicobacter pylori ferritin are not needed for nanoparticle formation but may be critical for proper folding and oligomerization when appended to envelope. Thus, constructs were designed with and without the leucine, serine, and lysine amino acids following the glycine-serine linker. In some embodiments, a linker length that is suitable for formation of envelope nanoparticles when ferritin is appended to most envelopes is used. Any suitable linker between the envelope and ferritin could be used, so long as the fusion protein is expressed and the trimer is formed.

Another approach to multimerize expression constructs uses staphylococcus Sortase A transpeptidase ligation to conjugate inventive envelope trimers, for e.g. but not limited to cholesterol. The trimers can then be embedded into liposomes via the conjugated cholesterol. To conjugate the trimer to cholesterol either a C-terminal LPXTG tag or a N-terminal pentaglycine repeat tag is added to the envelope trimer gene. Cholesterol is also synthesized with these two tags. Sortase A is then used to covalently bond the tagged envelope to the cholesterol.

The sortase A-tagged trimer protein can also be used to conjugate the trimer to other peptides, proteins, or fluorescent labels. In non-limiting embodiments, the sortase A tagged trimers are conjugated to ferritin to form nanoparticles. Any suitable ferritin can be used.

The invention provides design of envelopes and trimer designs wherein the envelope comprises a linker which permits addition of a lipid, such as but not limited to cholesterol, via a Sortase A reaction. See e.g., Tsukiji, S. and Nagamune, T. (2009), Sortase-Mediated Ligation: A Gift from Gram-Positive Bacteria to Protein Engineering. ChemBioChem, 10: 787-798. doi:10.1002/cbic.200800724; Proft, T. Sortase-mediated protein ligation: an emerging biotechnology tool for protein modification and immobilisation. Biotechnol Lett (2010) 32: 1. doi:10.1007/s10529-009-0116-0; Lena Schmohl, Dirk Schwarzer, Sortase-mediated ligations for the site-specific modification of proteins, Current Opinion in Chemical Biology, Volume 22, October 2014, Pages 122-128, ISSN 1367-5931, dx.doi.org/10.1016/j.cbpa.2014.09.020; Tabata et al. Anticancer Res. 2015 August; 35(8):4411-7; Pritz et al. J. Org. Chem. 2007, 72, 3909-3912.

The lipid modified envelopes and trimers could be formulated as liposomes. Any suitable liposome composition is contemplated.

The lipid modified and multimerized envelopes and trimers could be formulated as liposomes. Any suitable liposome composition is contemplated.

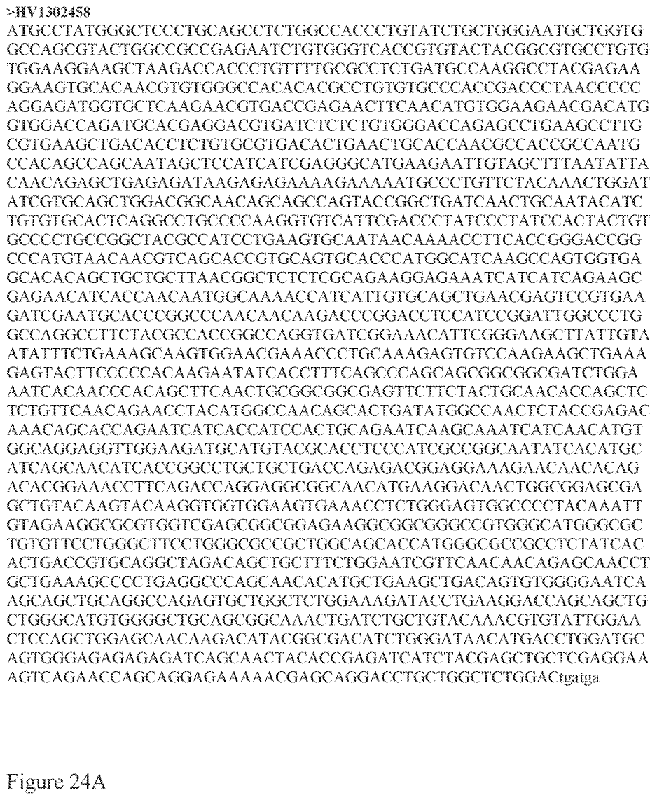

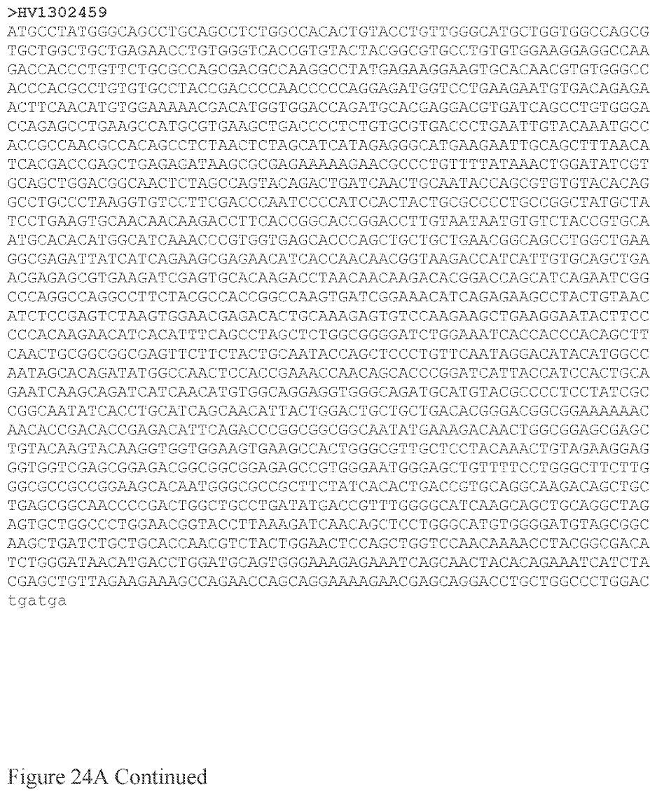

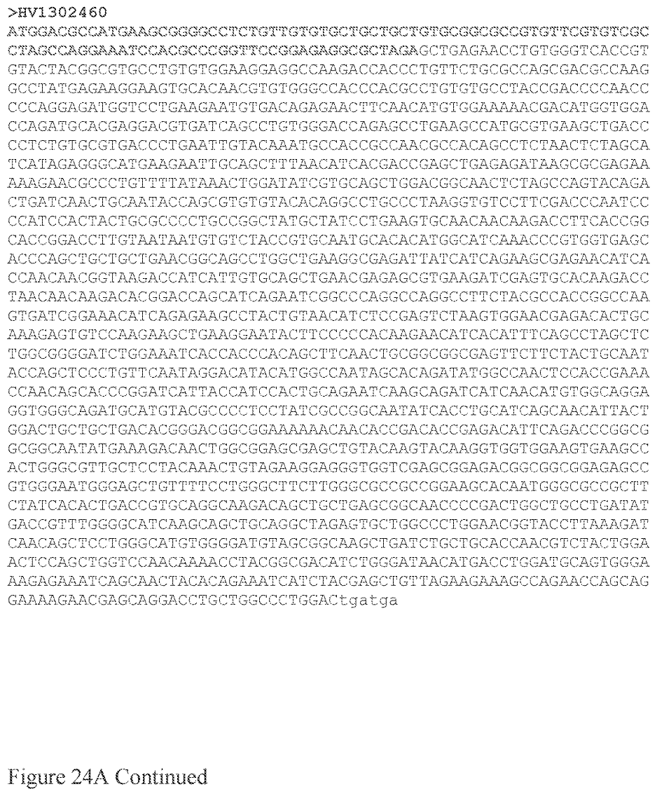

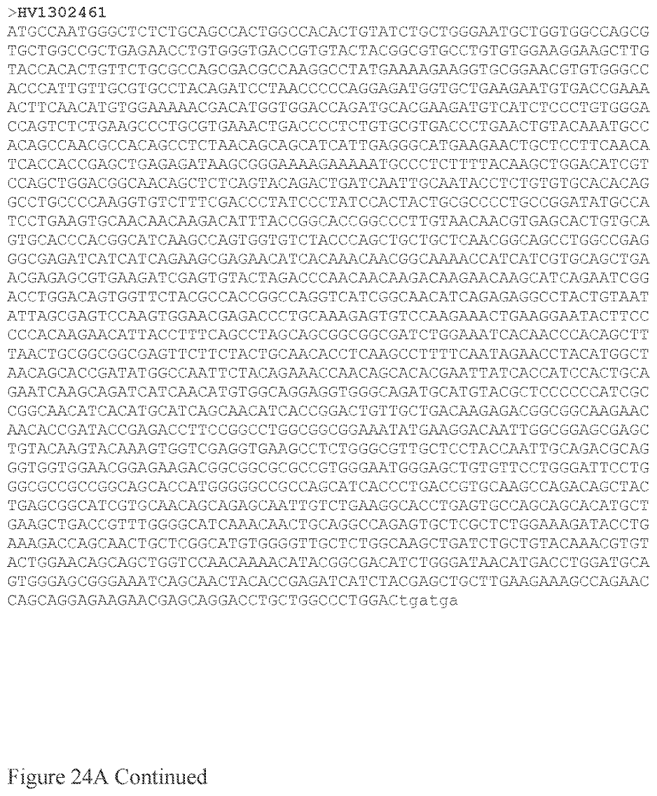

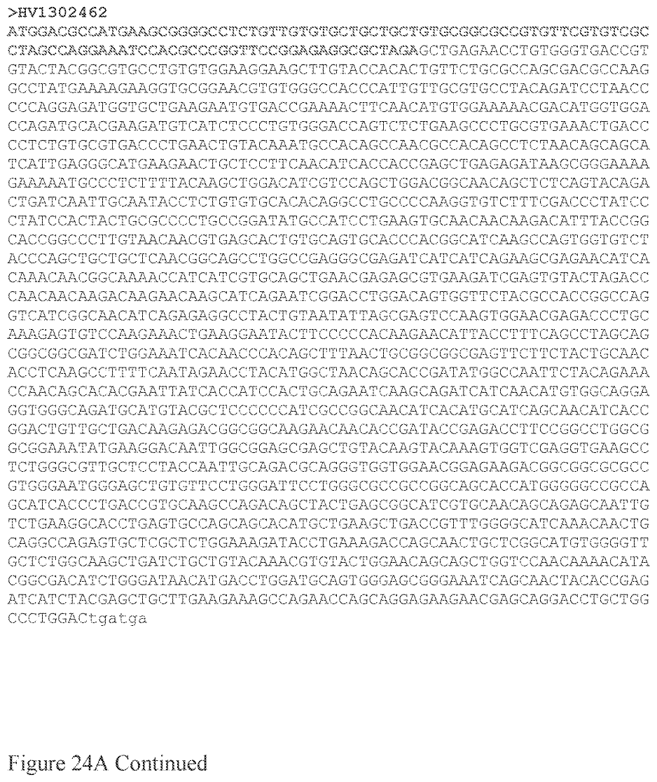

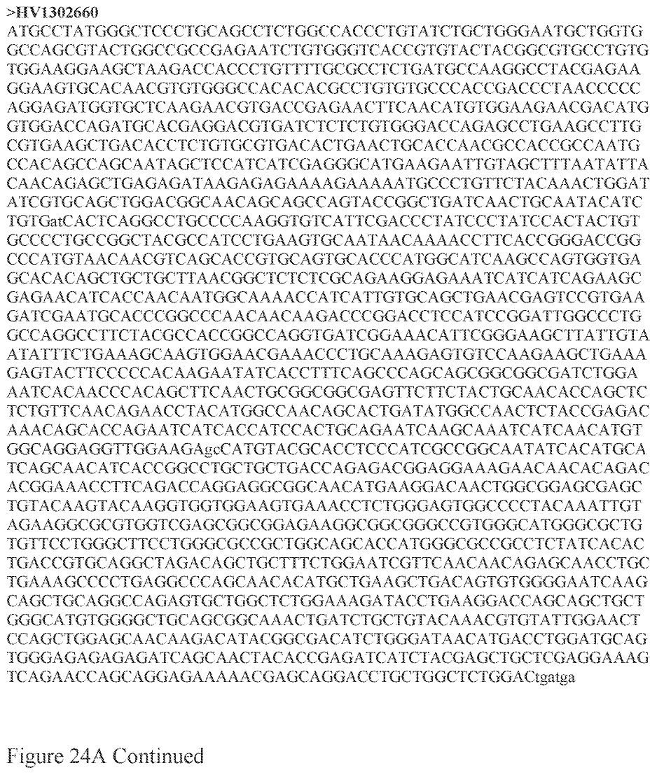

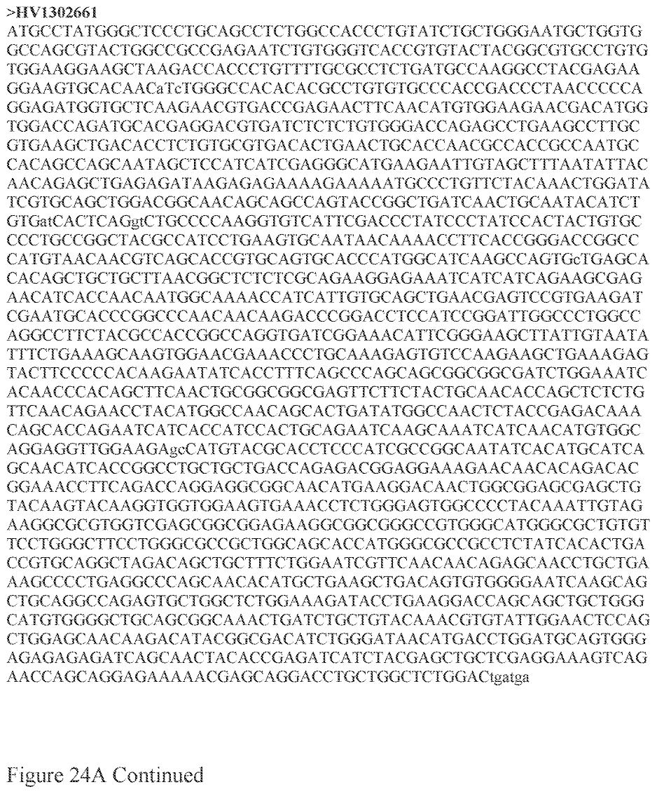

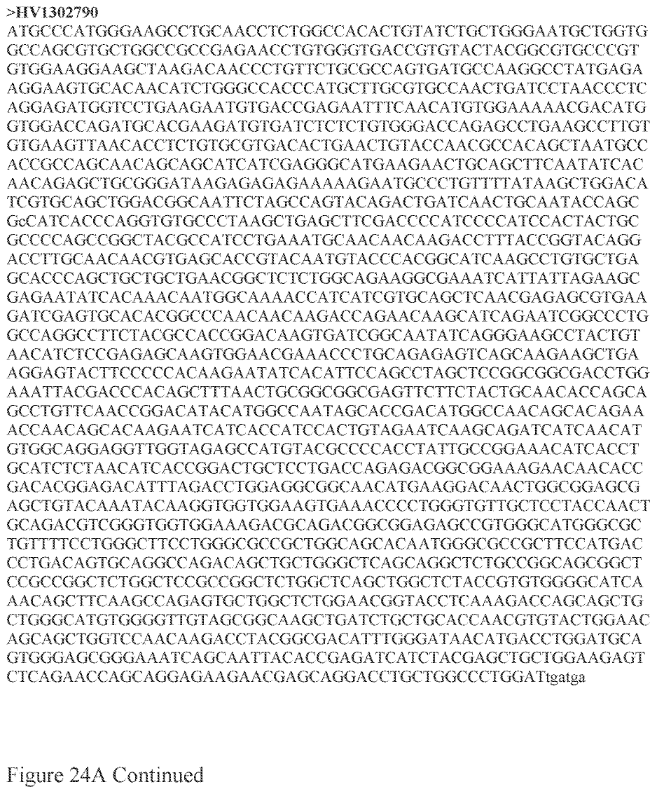

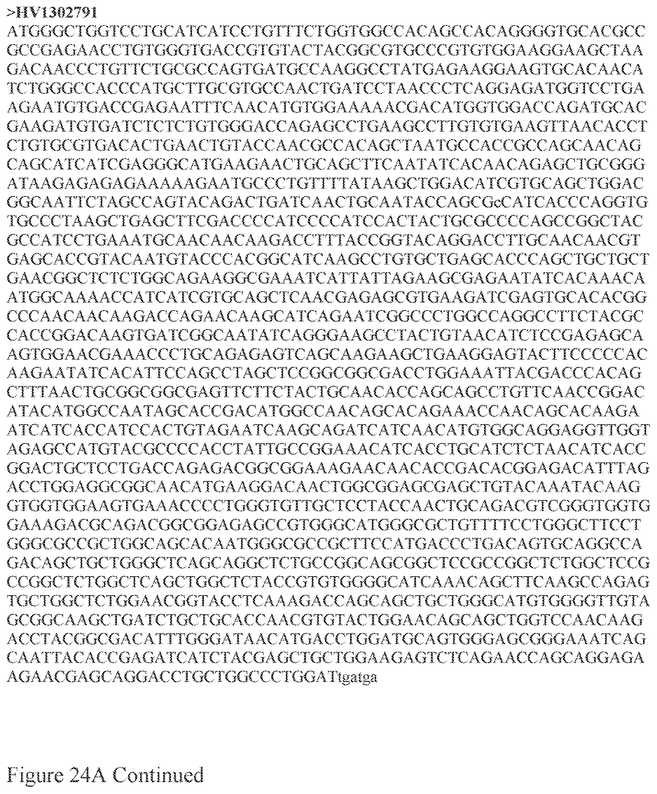

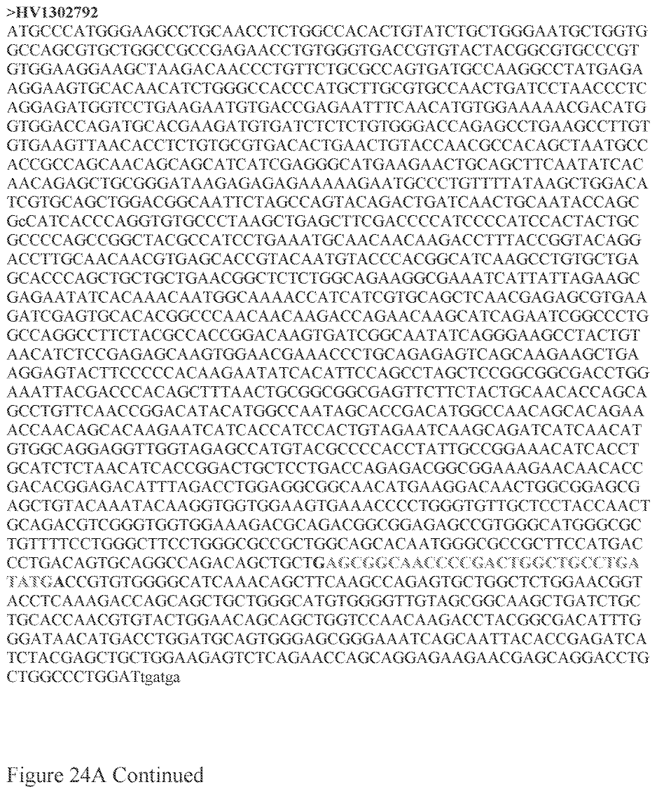

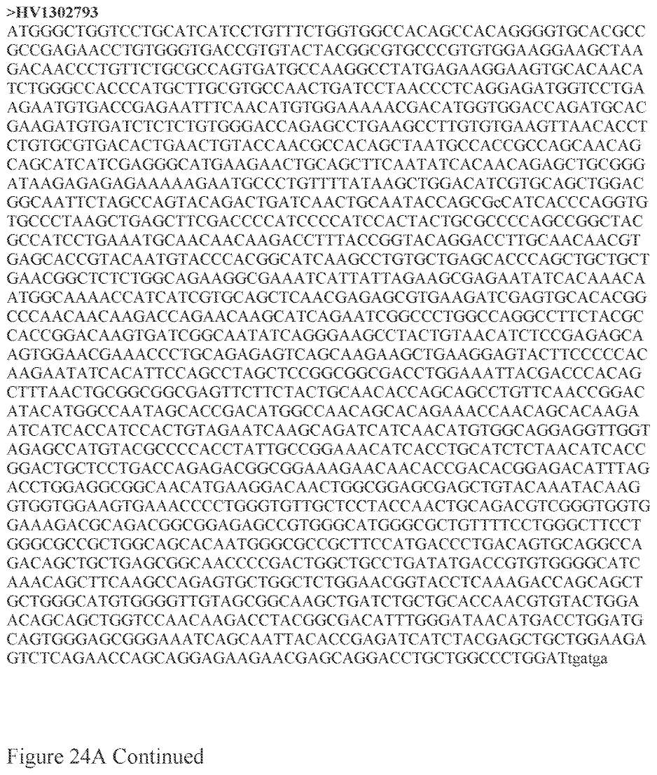

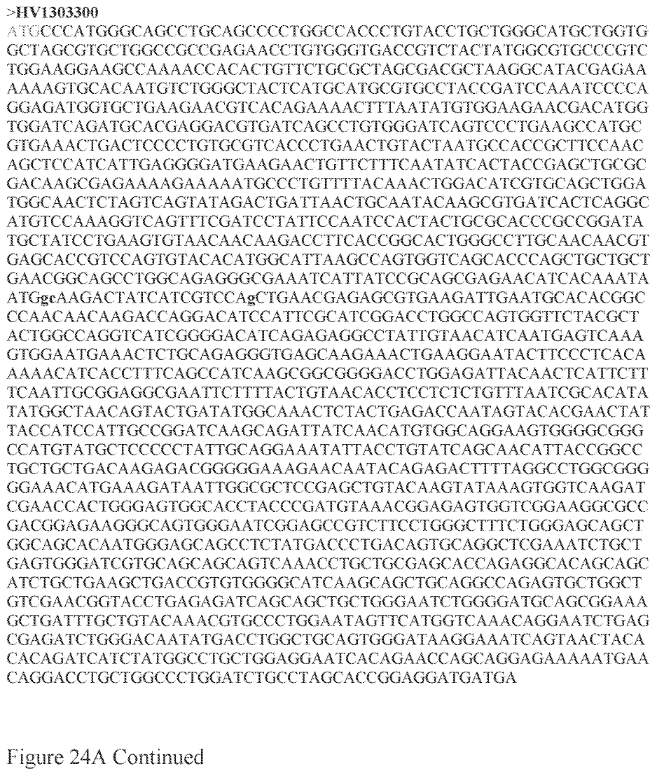

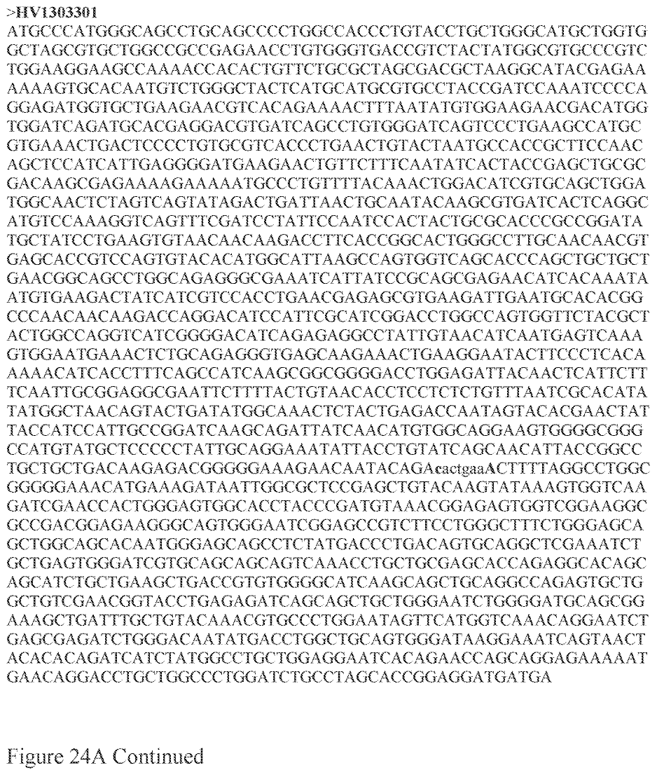

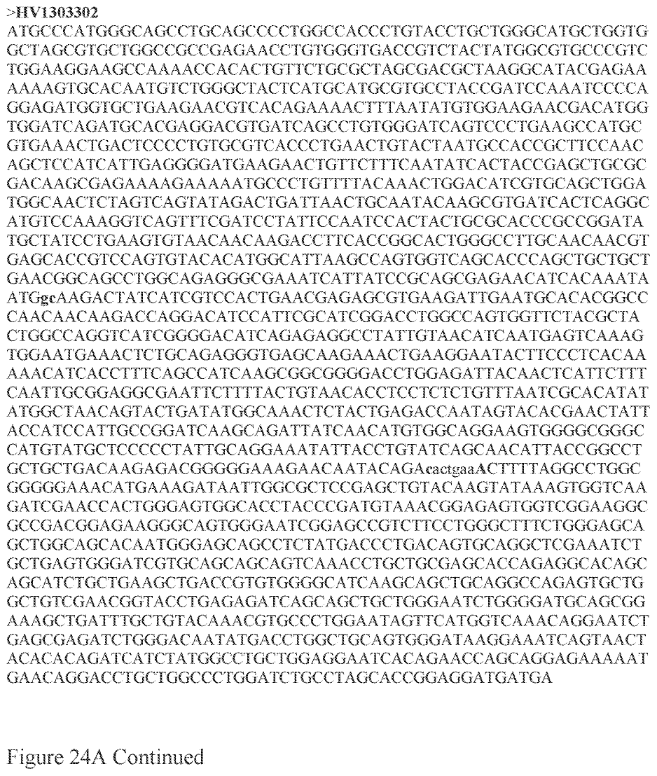

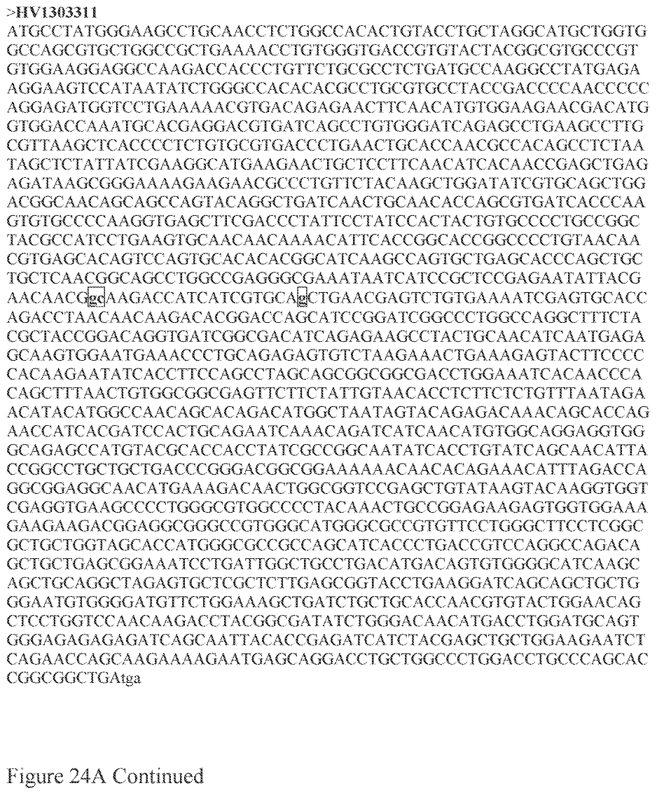

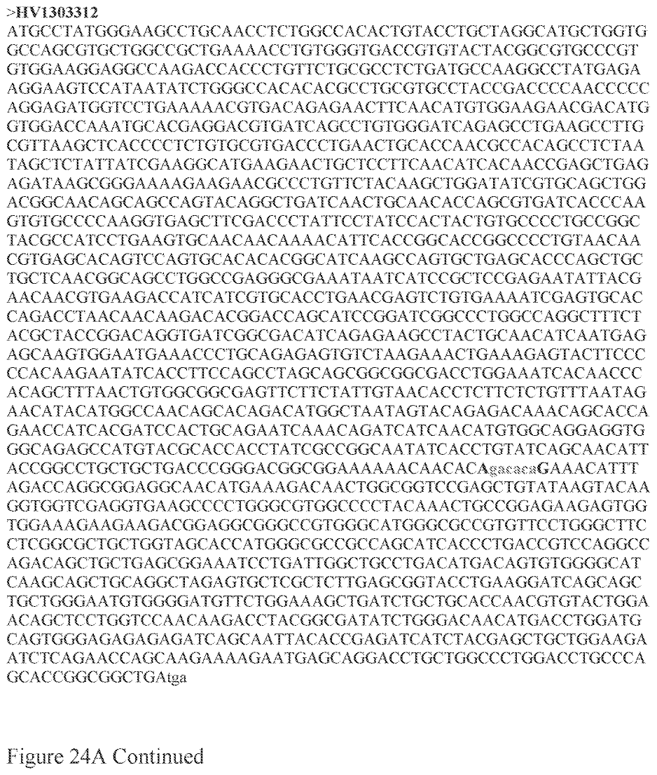

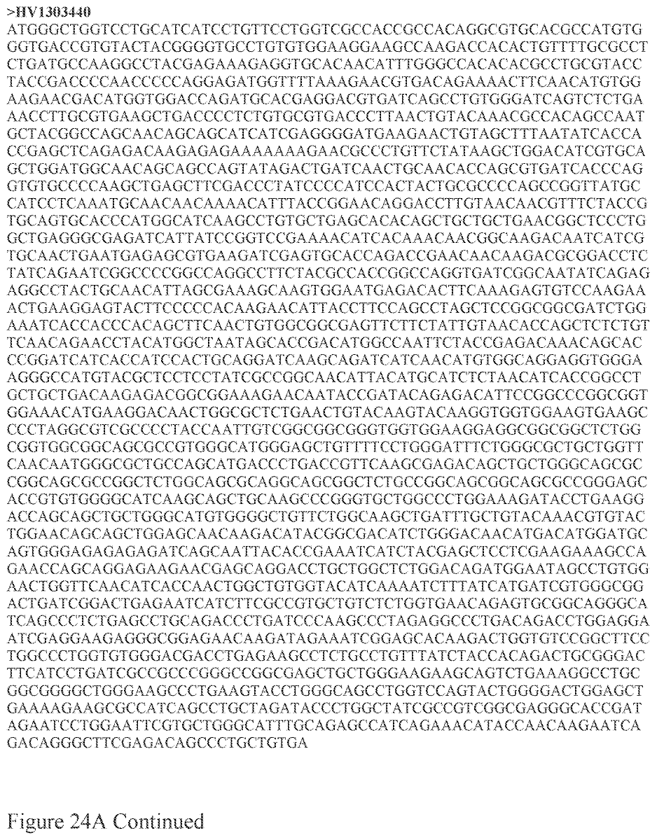

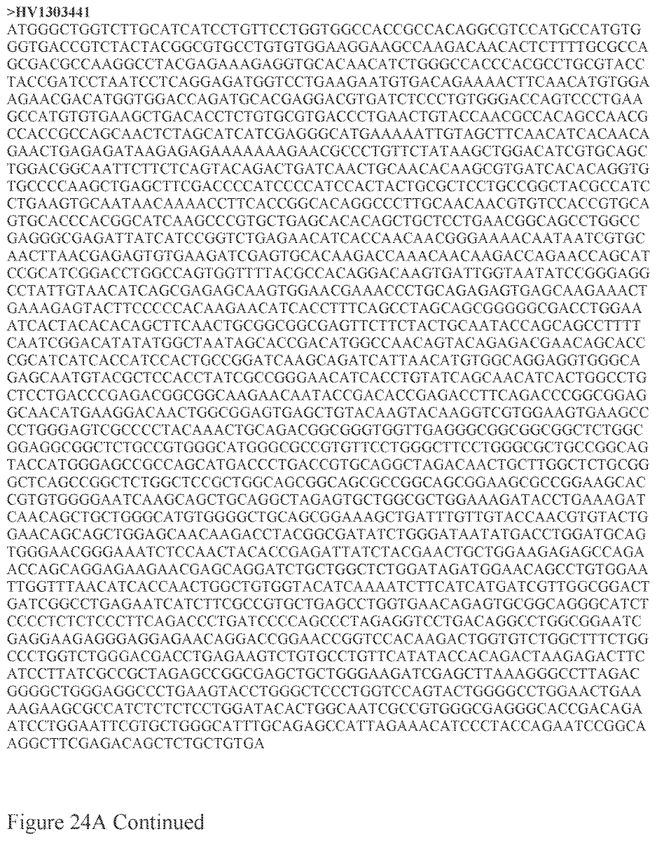

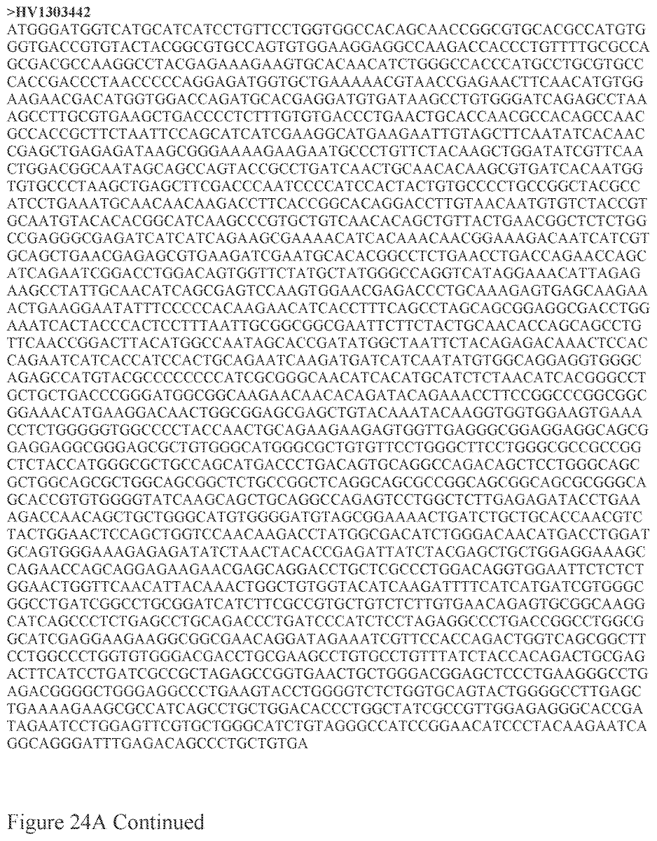

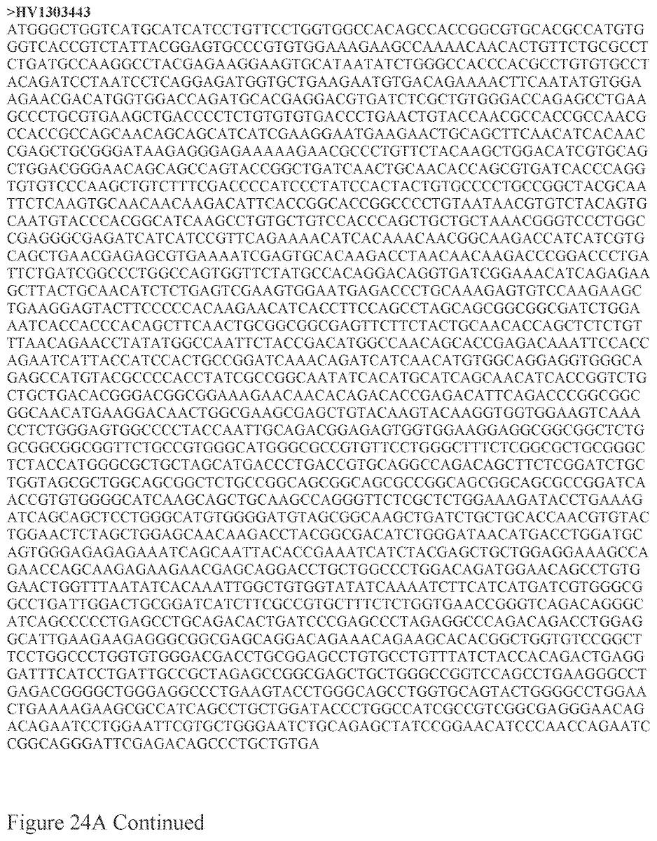

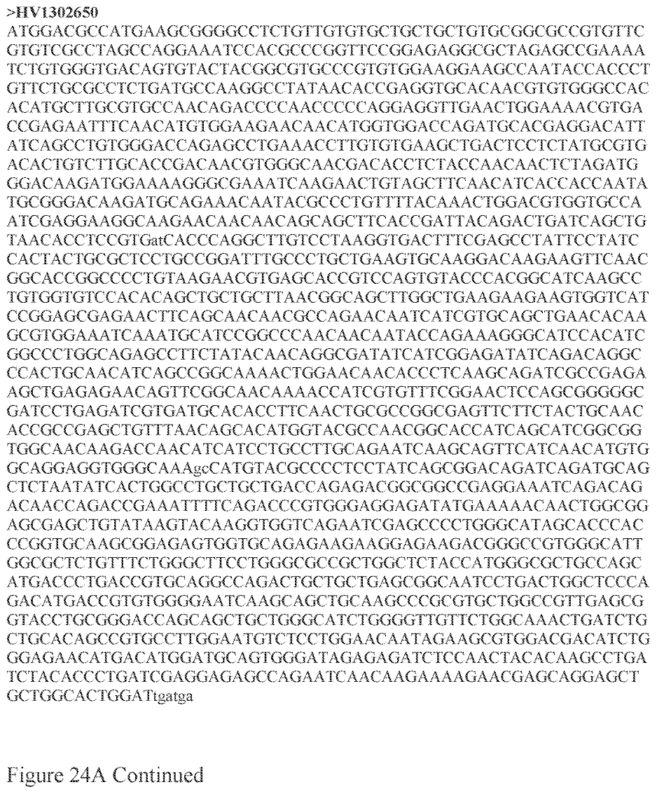

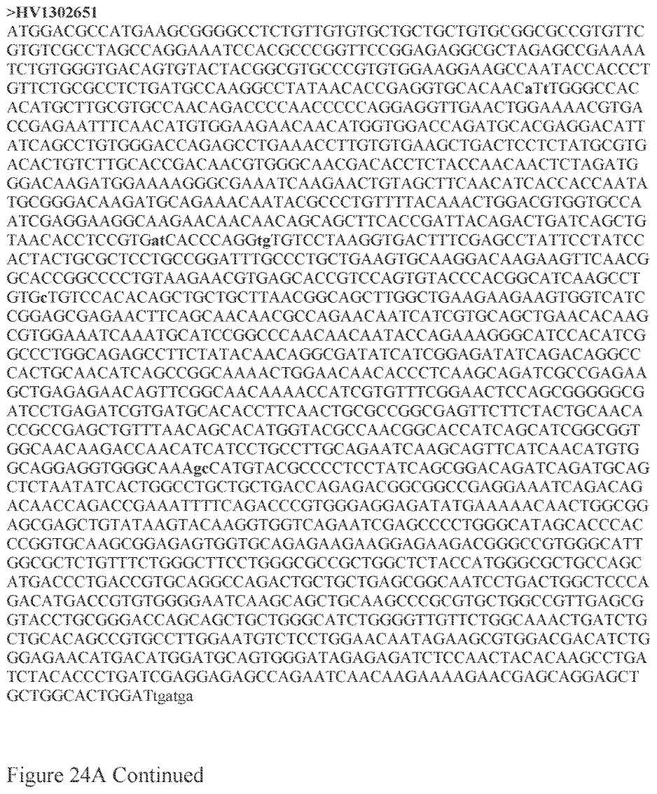

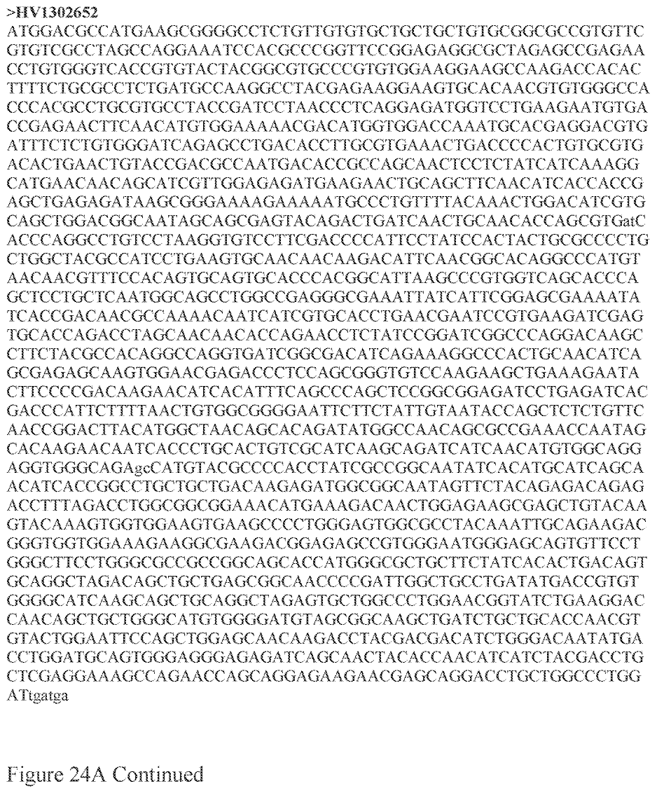

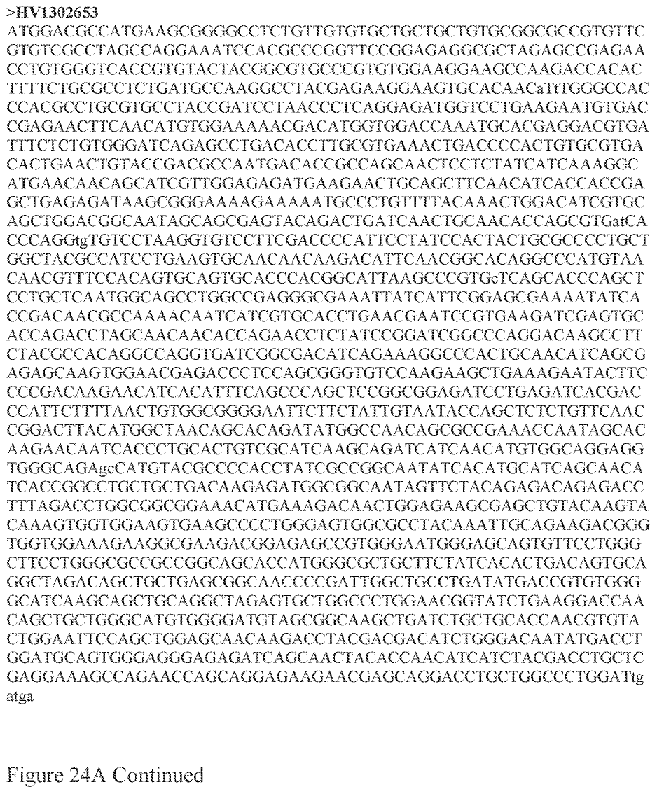

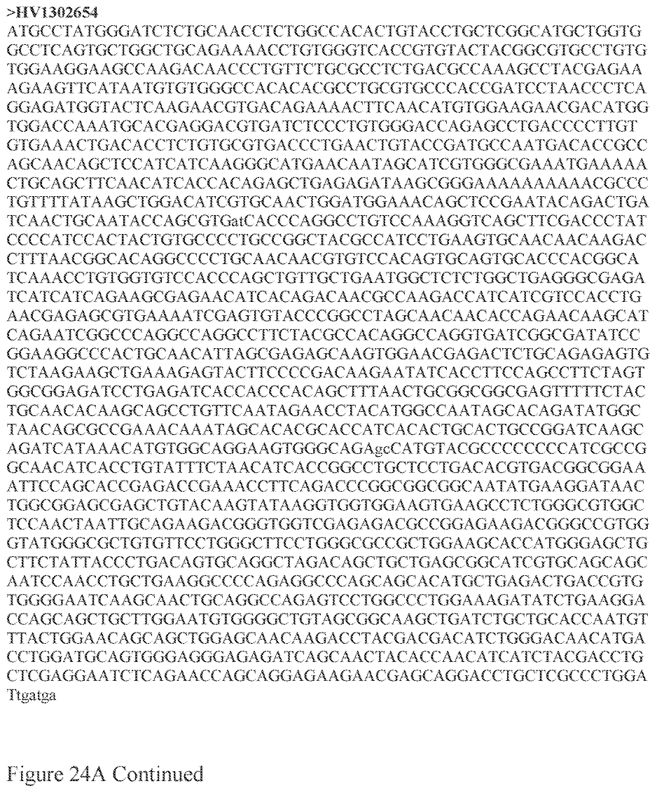

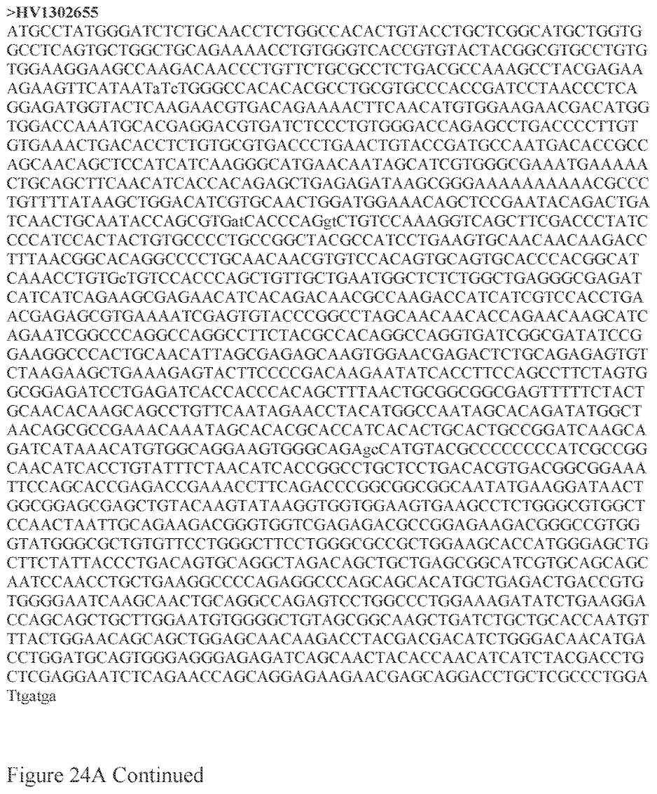

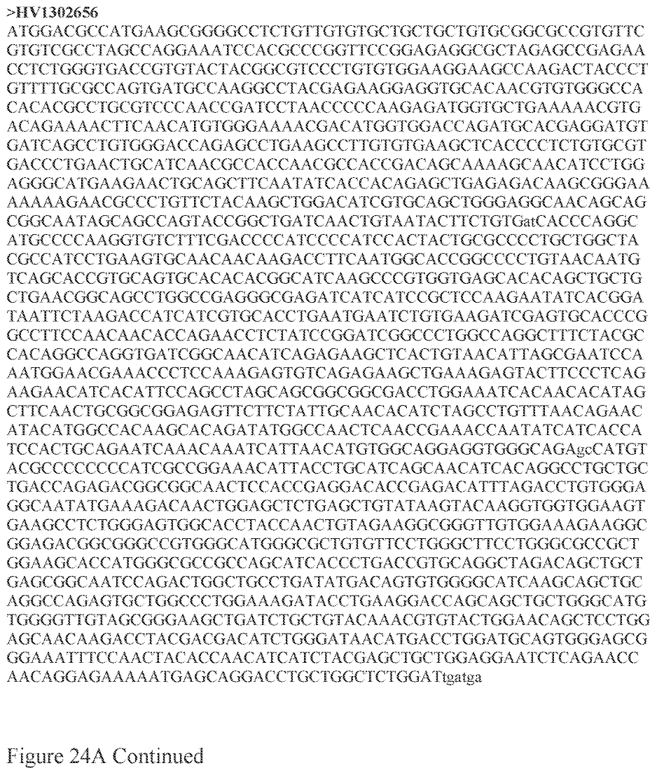

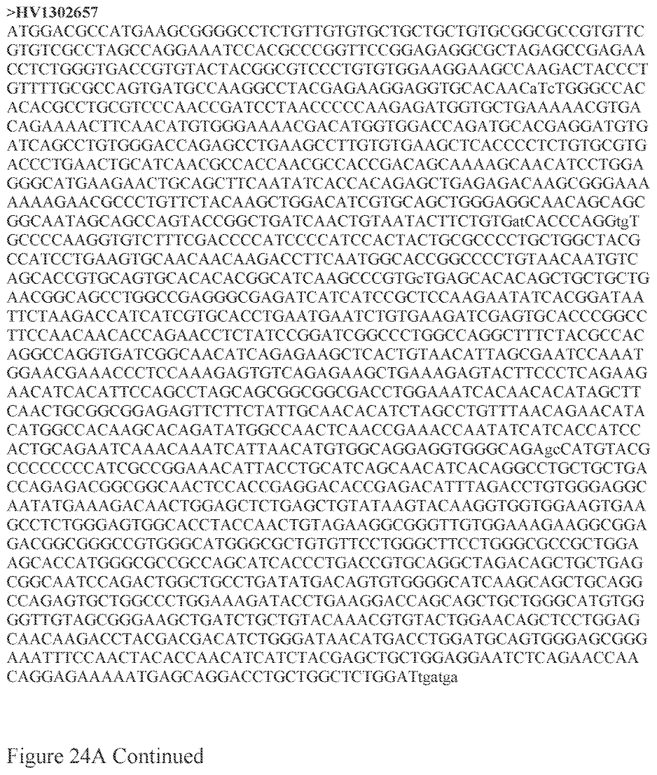

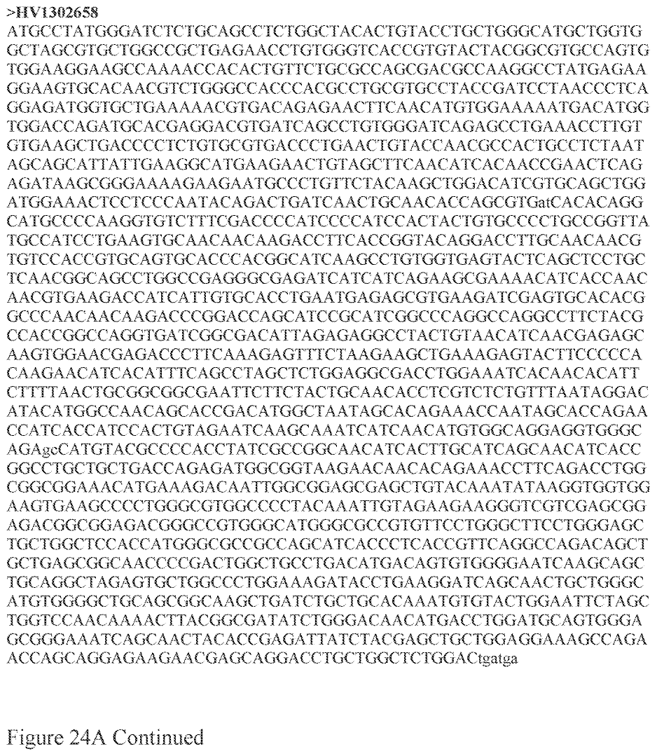

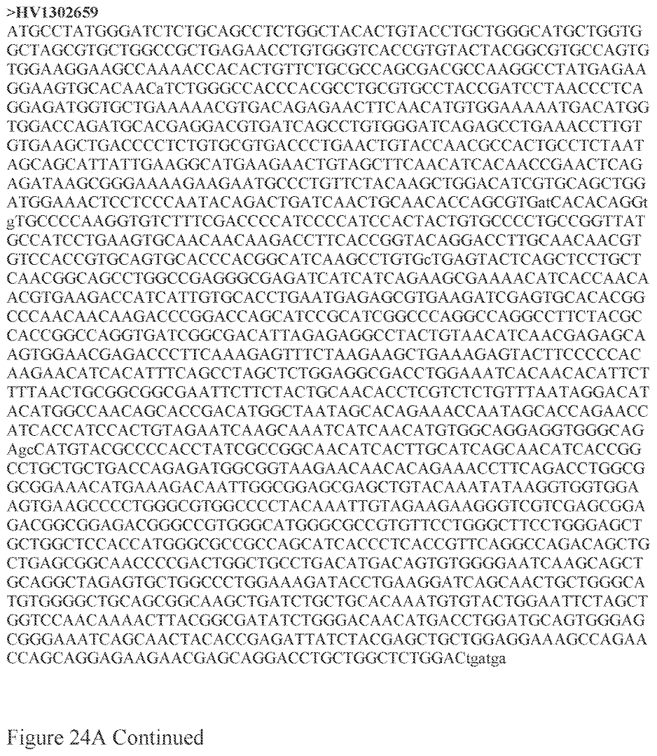

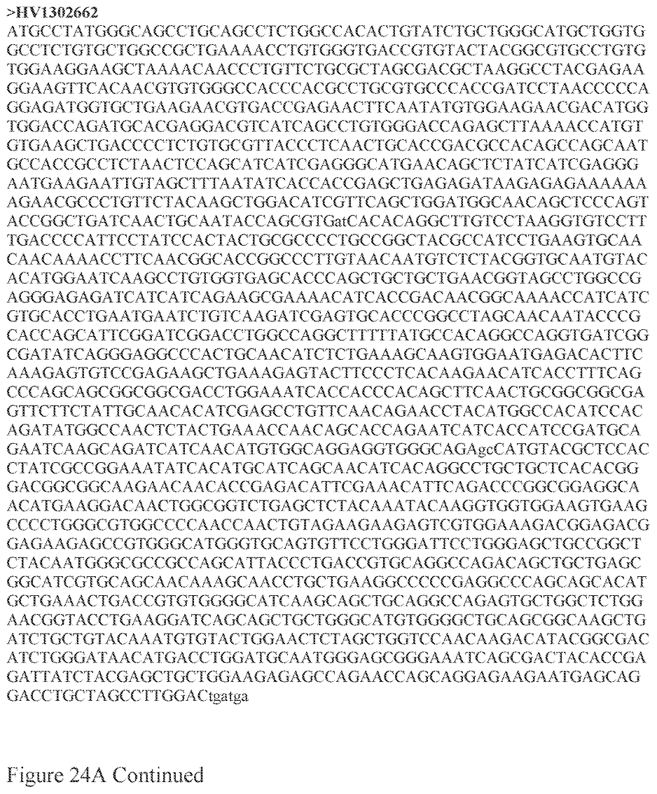

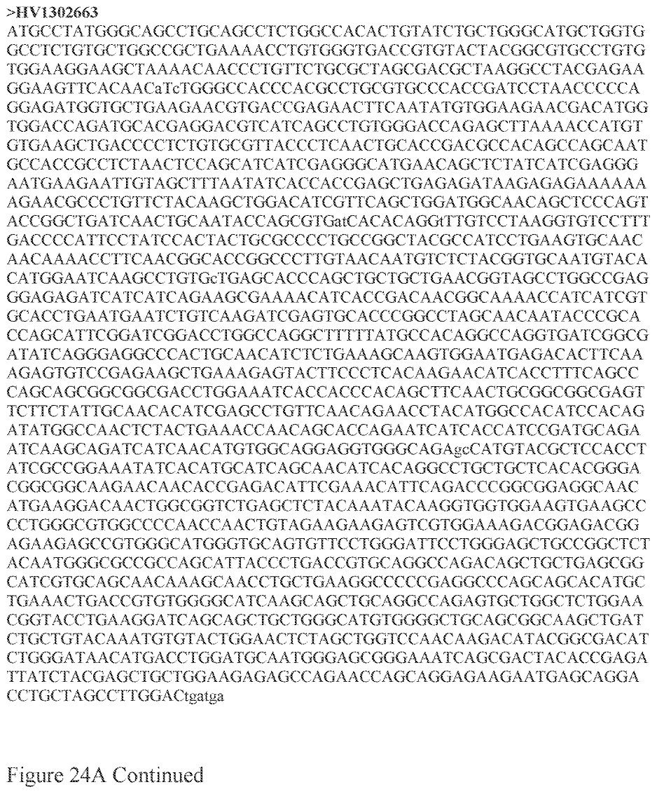

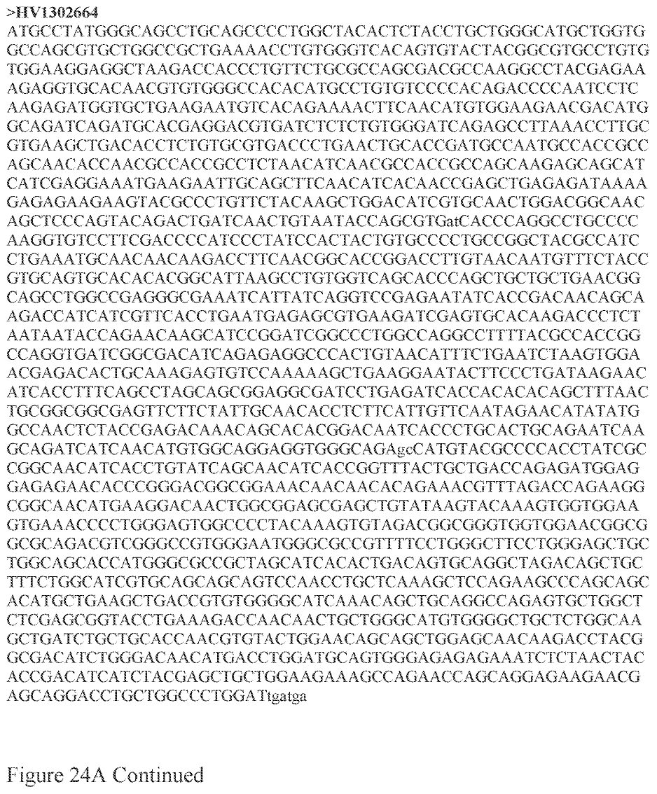

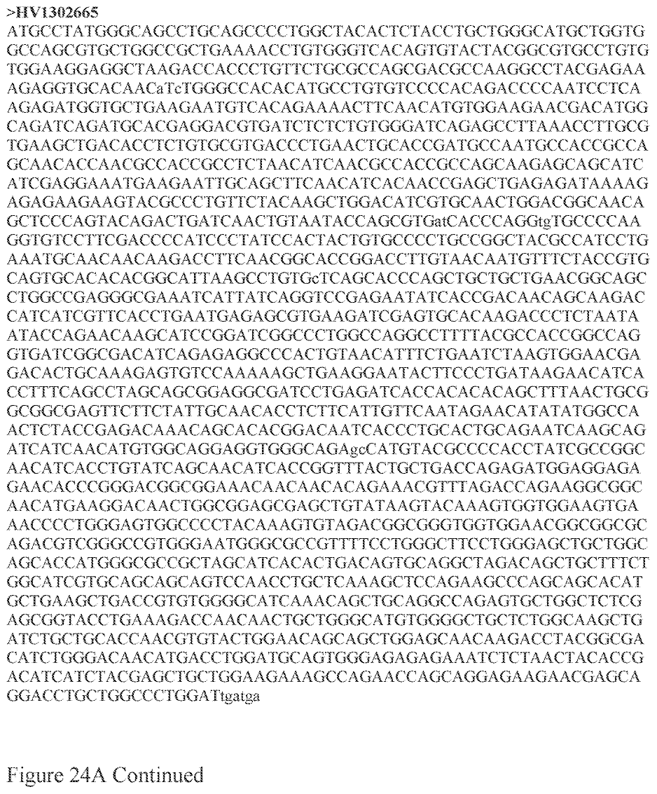

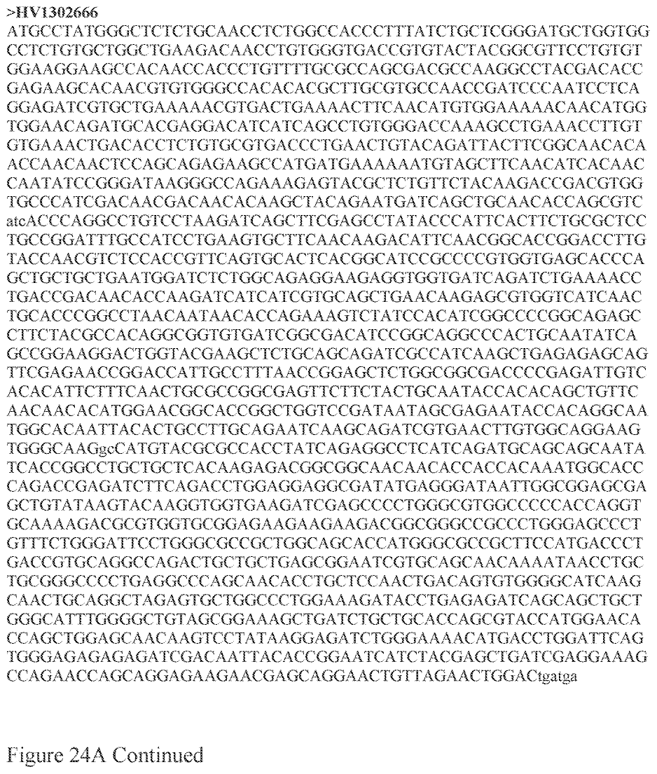

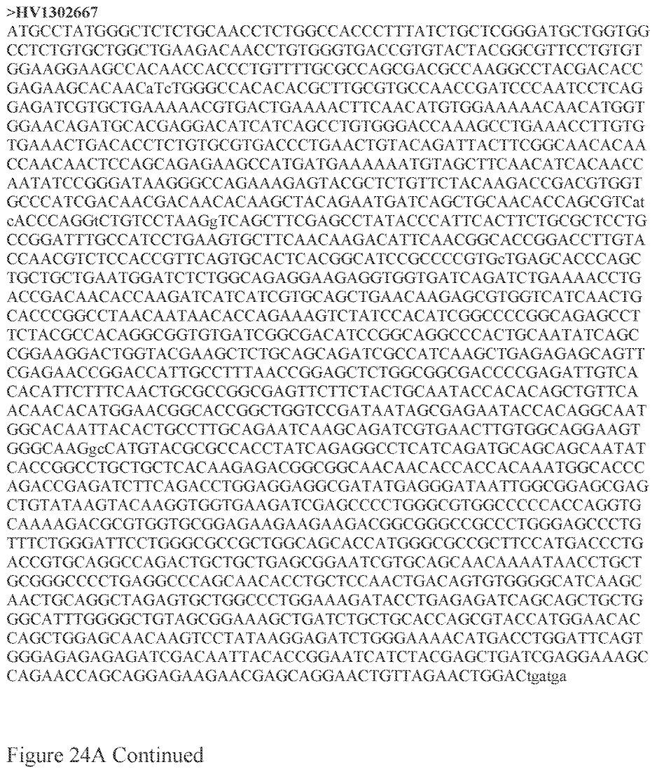

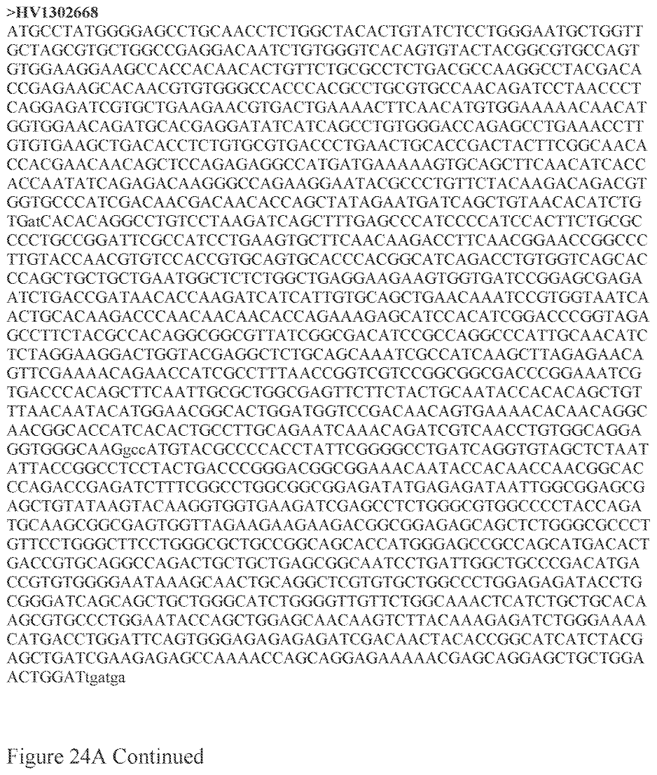

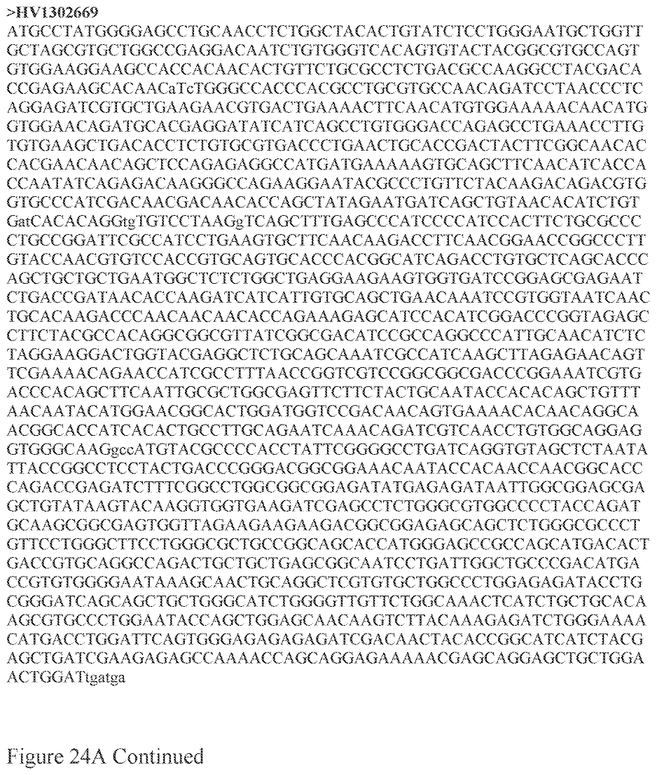

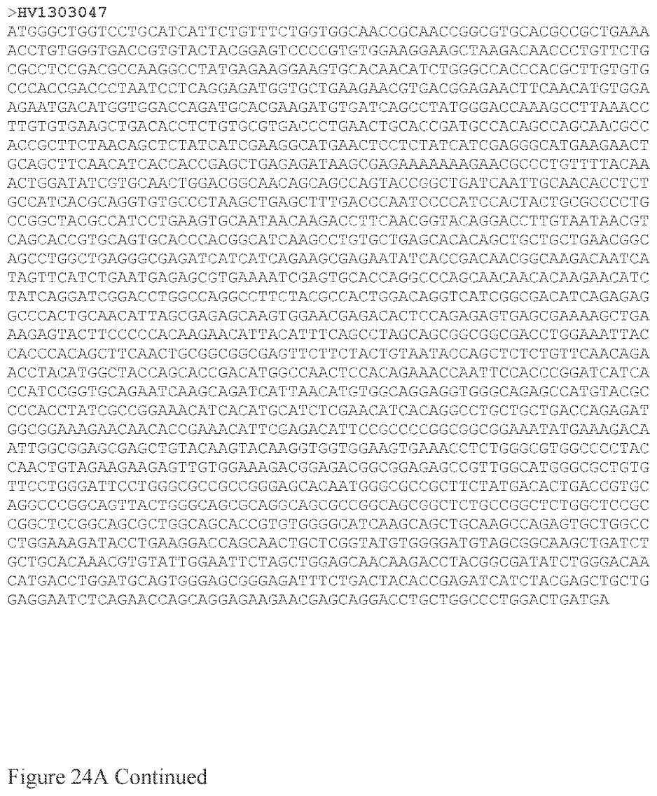

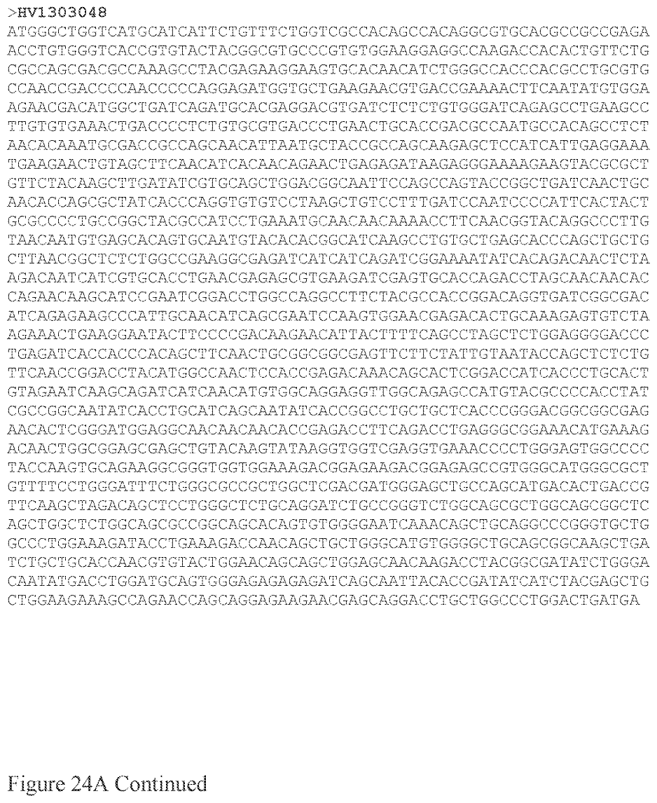

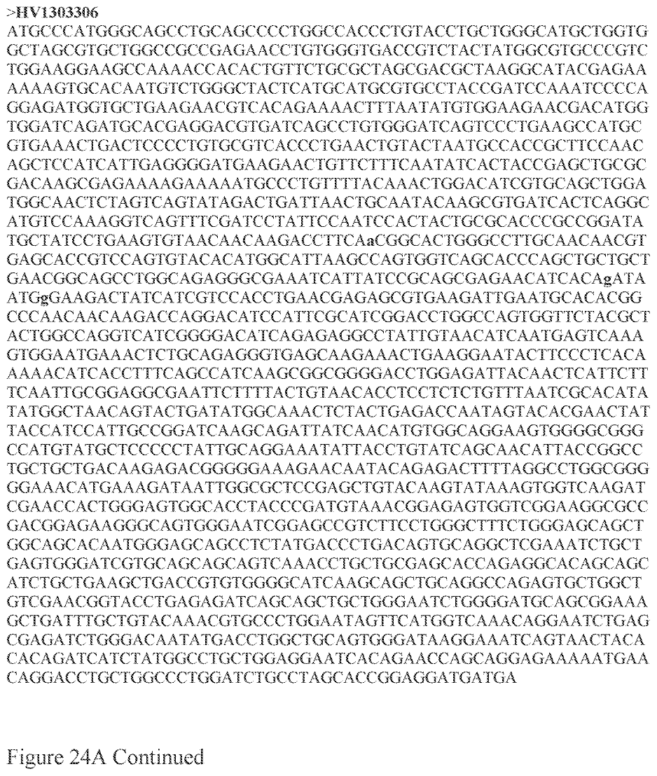

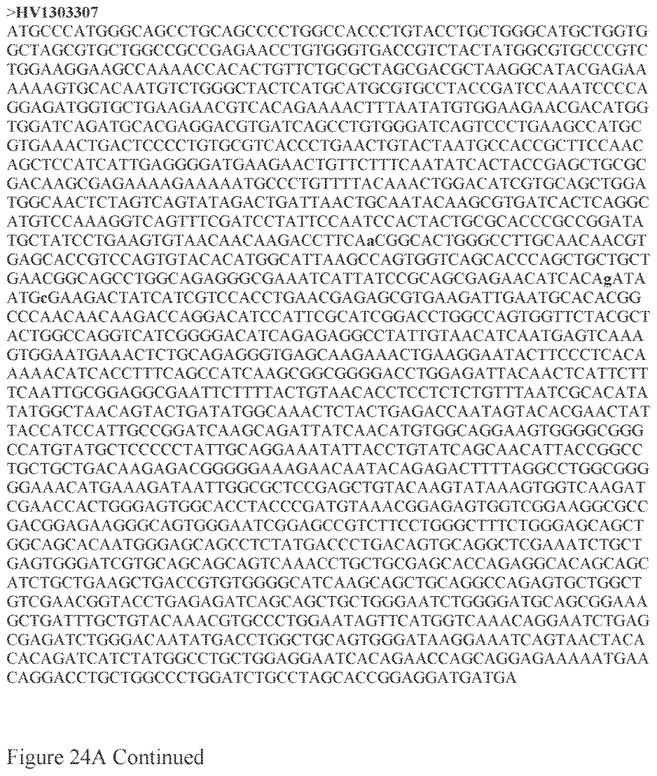

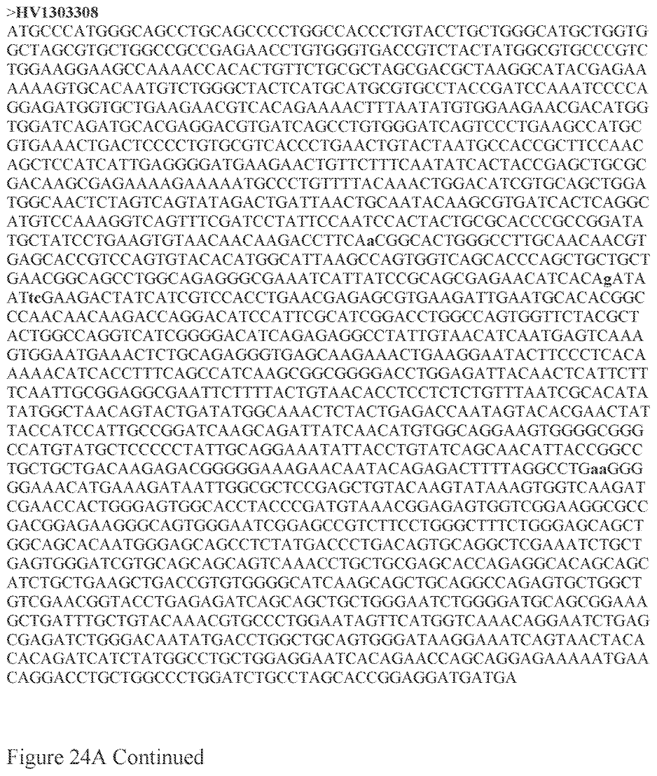

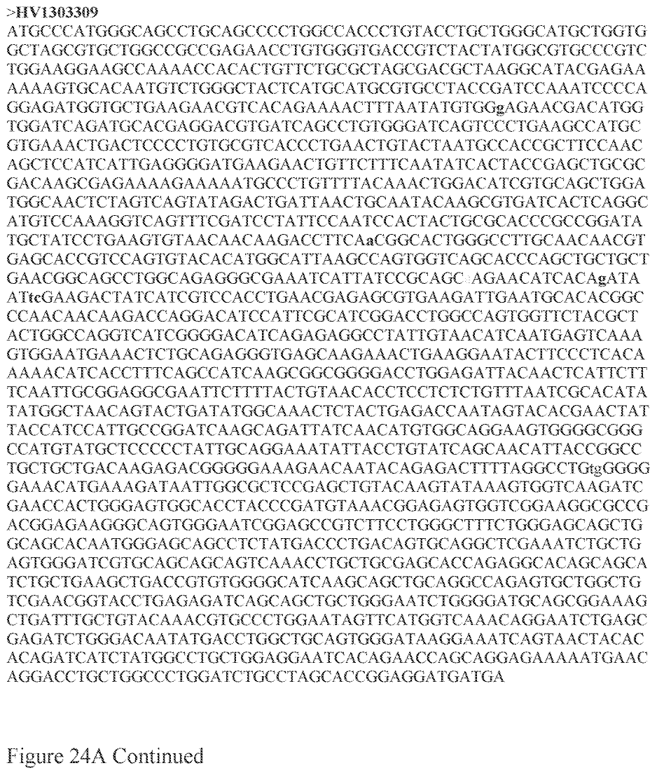

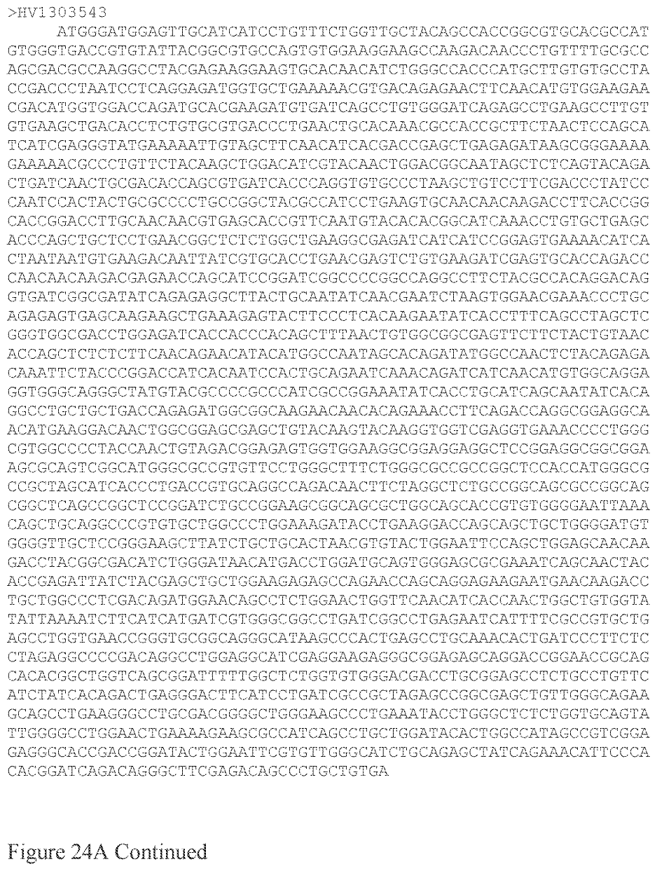

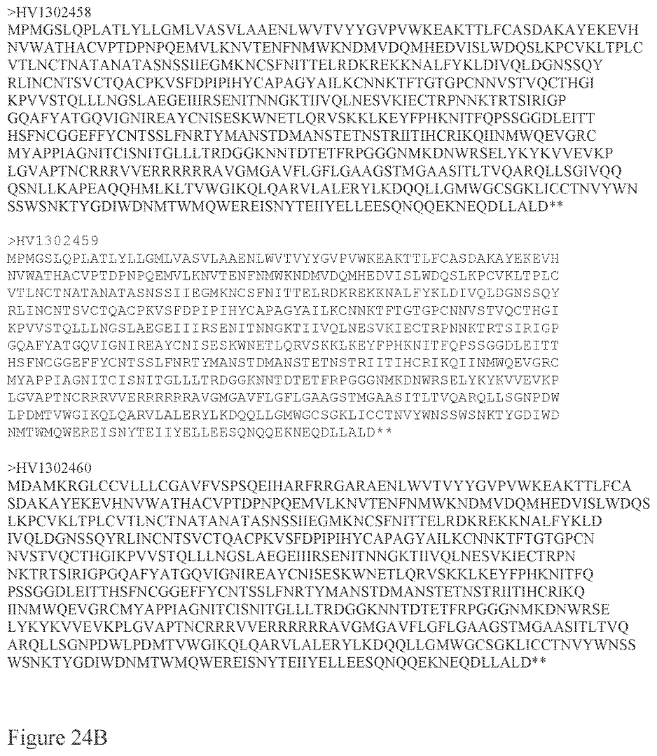

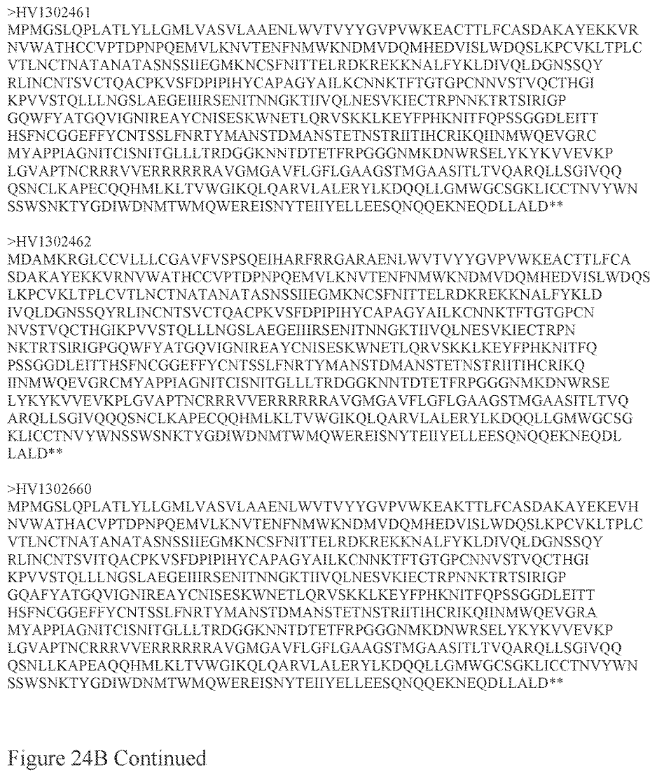

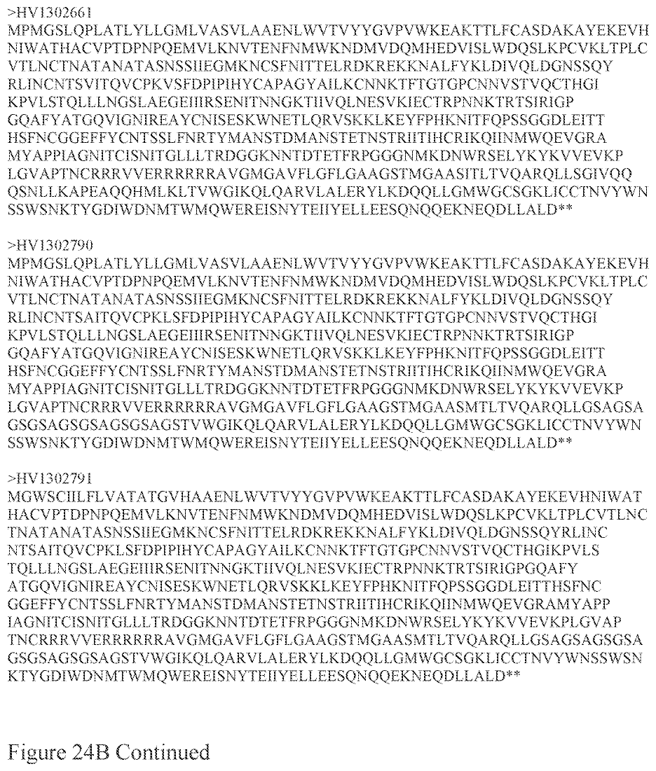

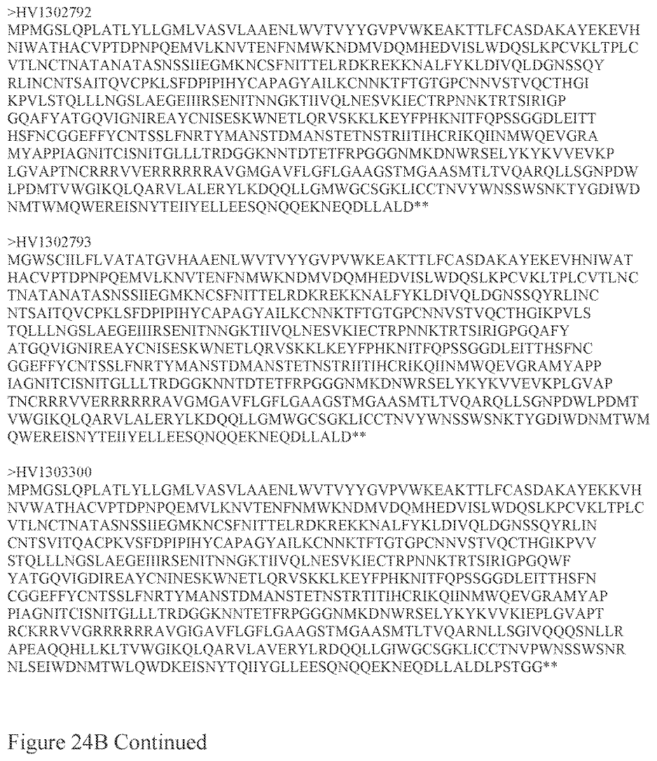

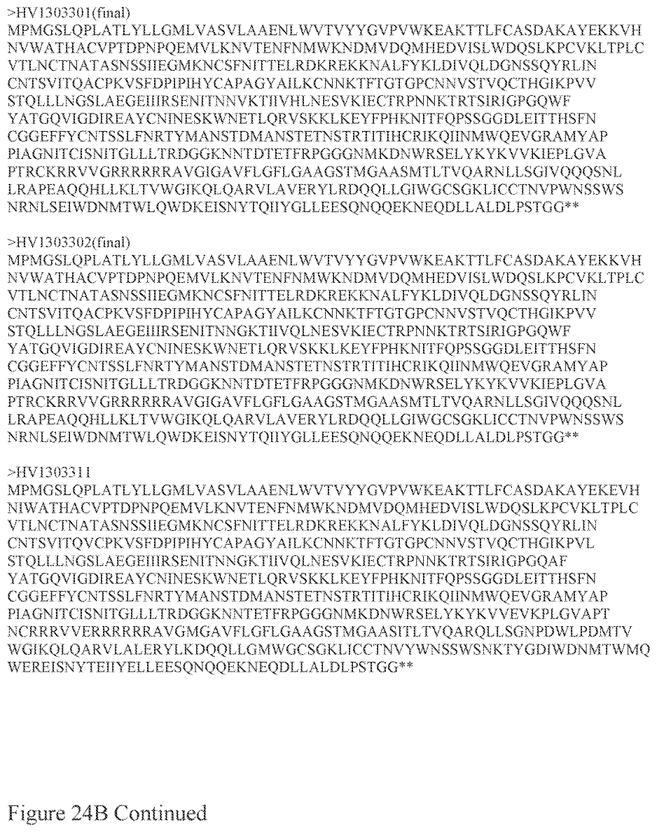

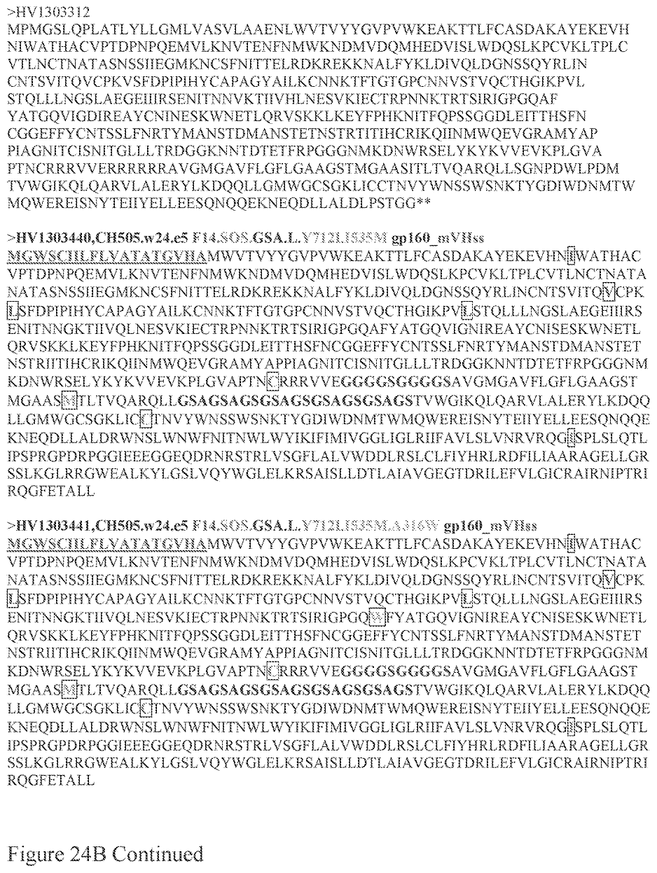

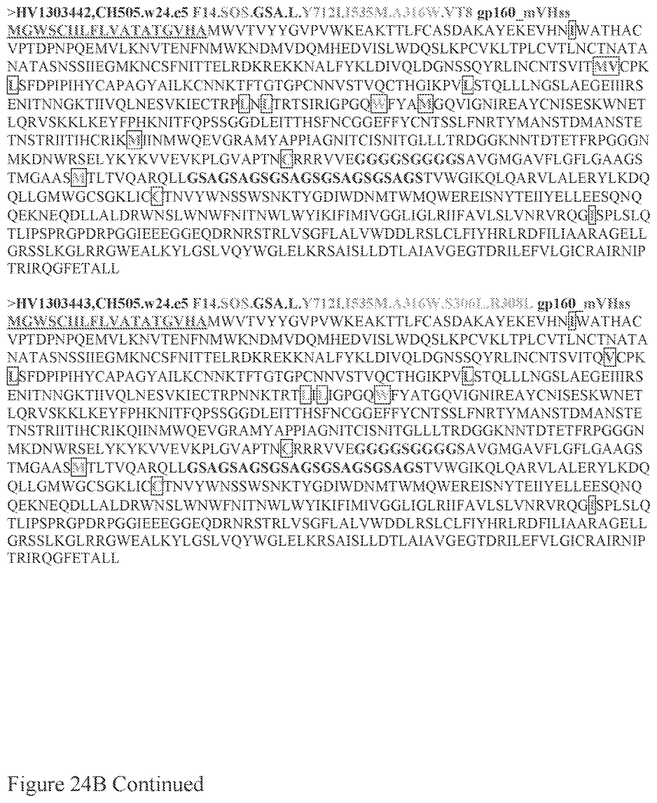

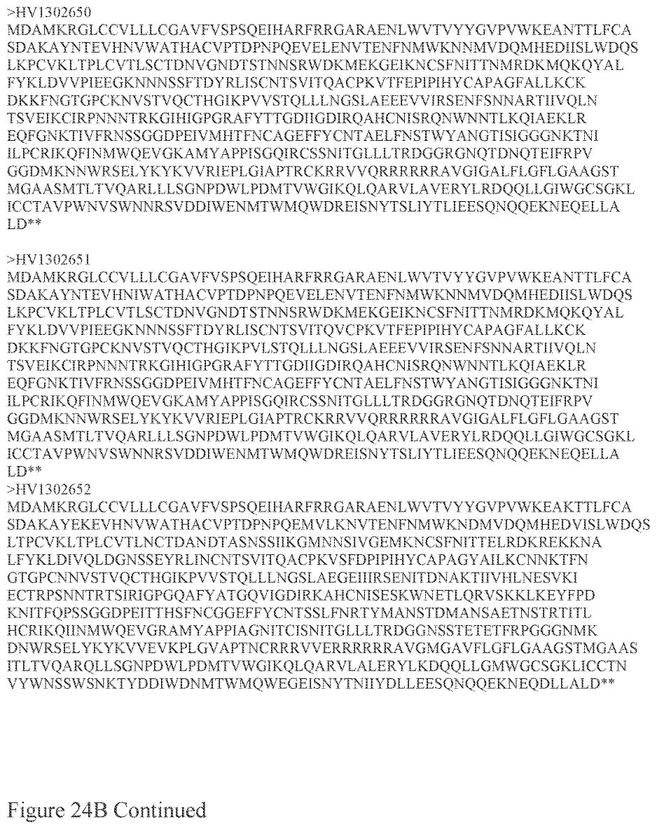

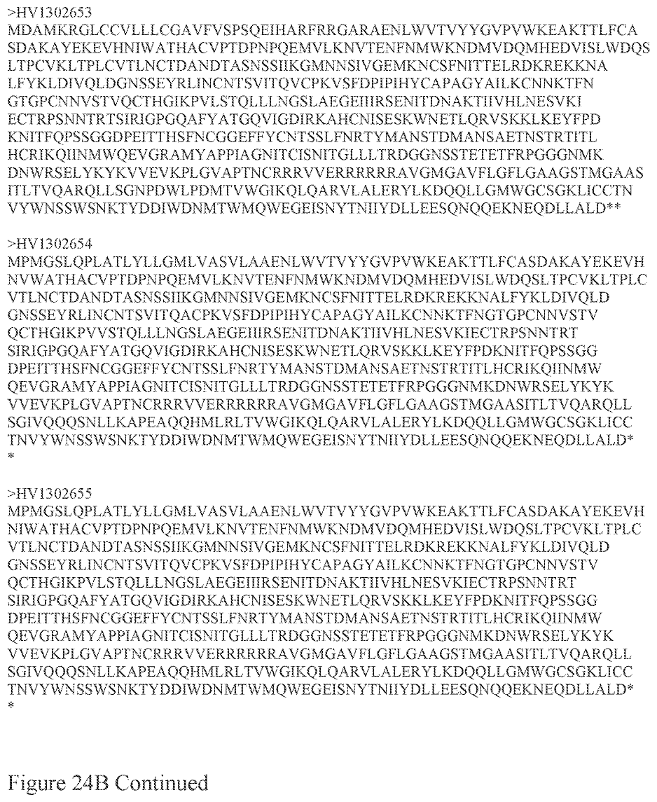

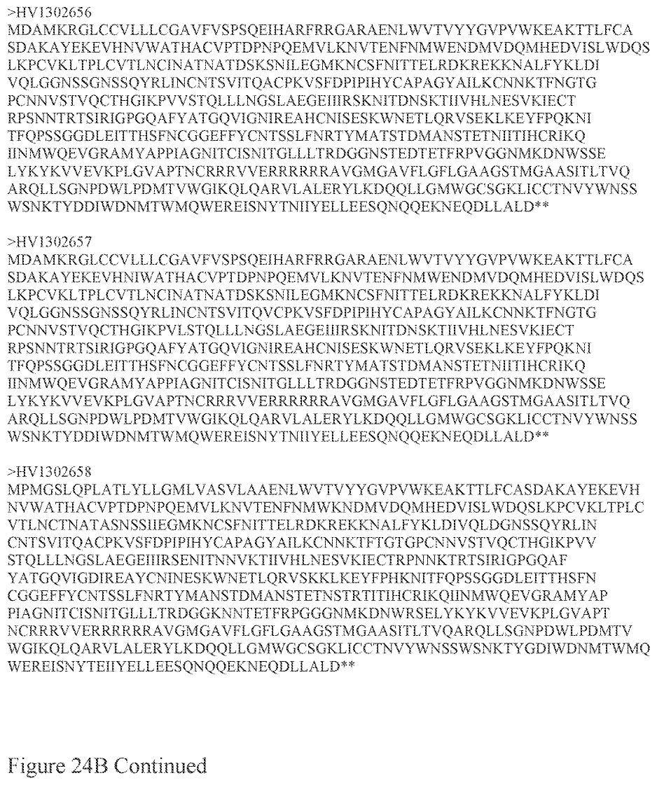

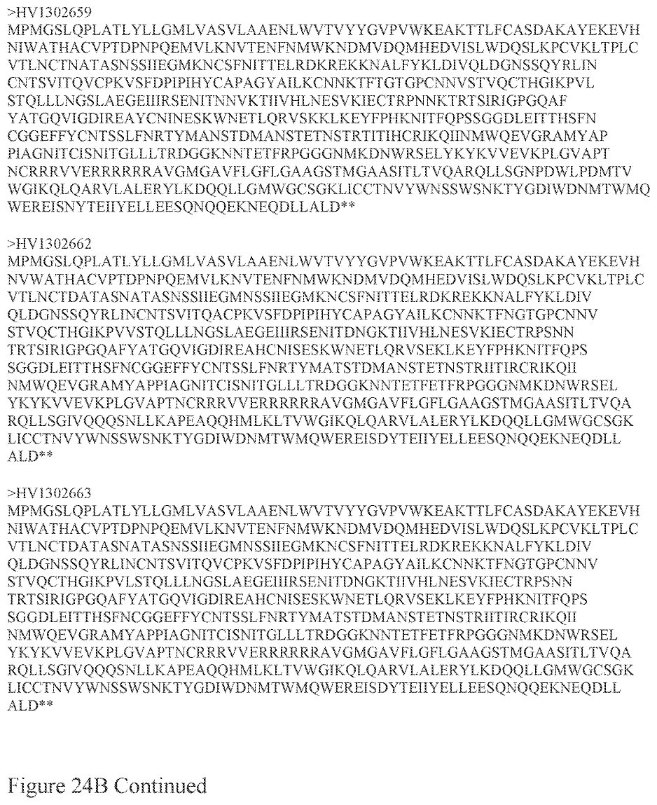

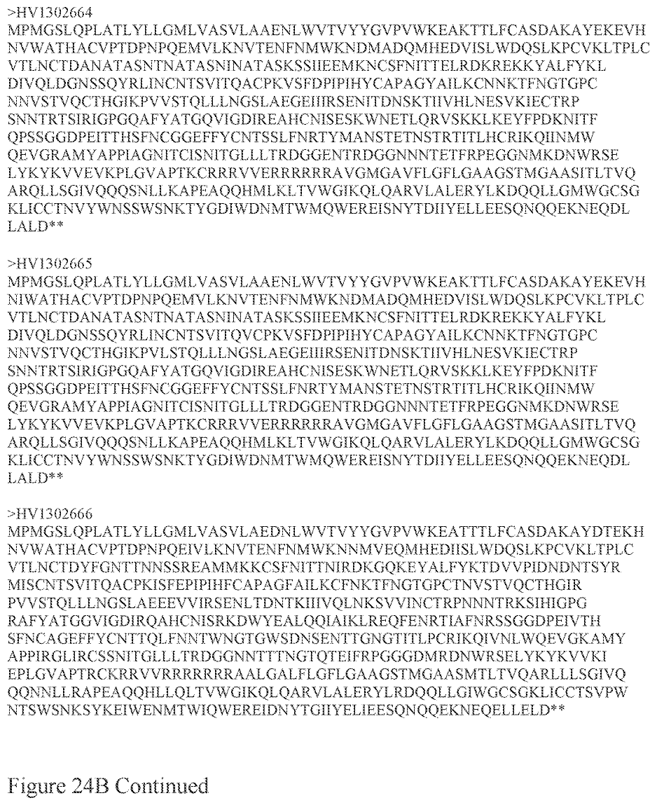

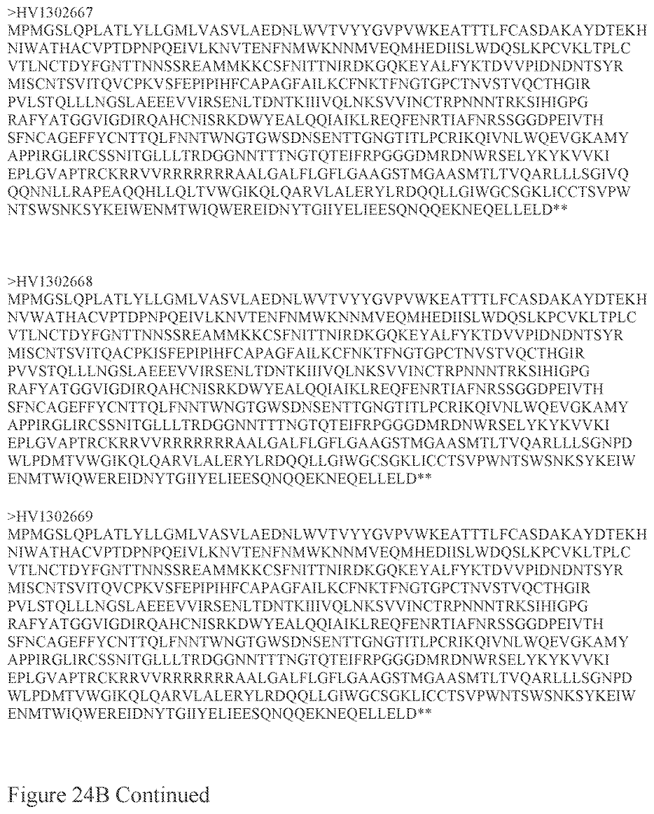

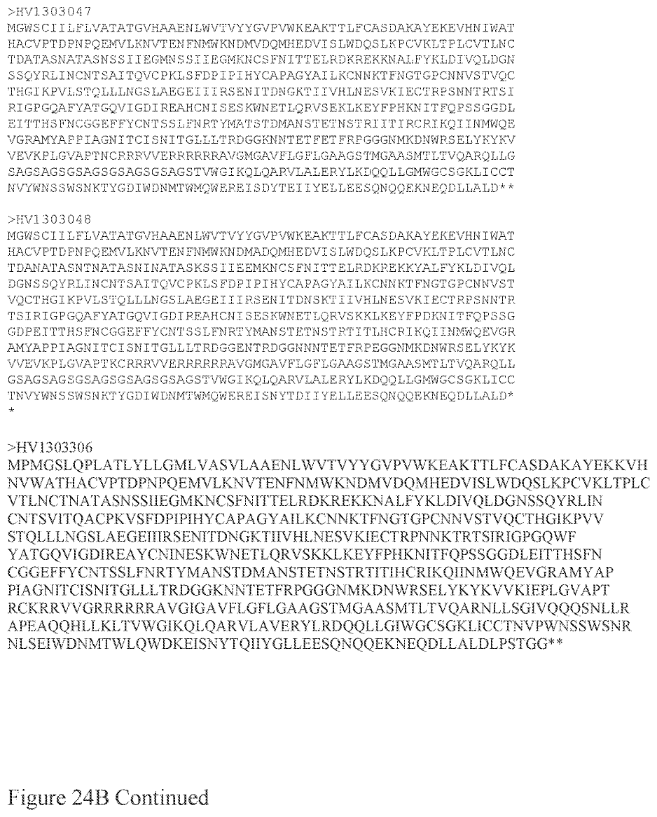

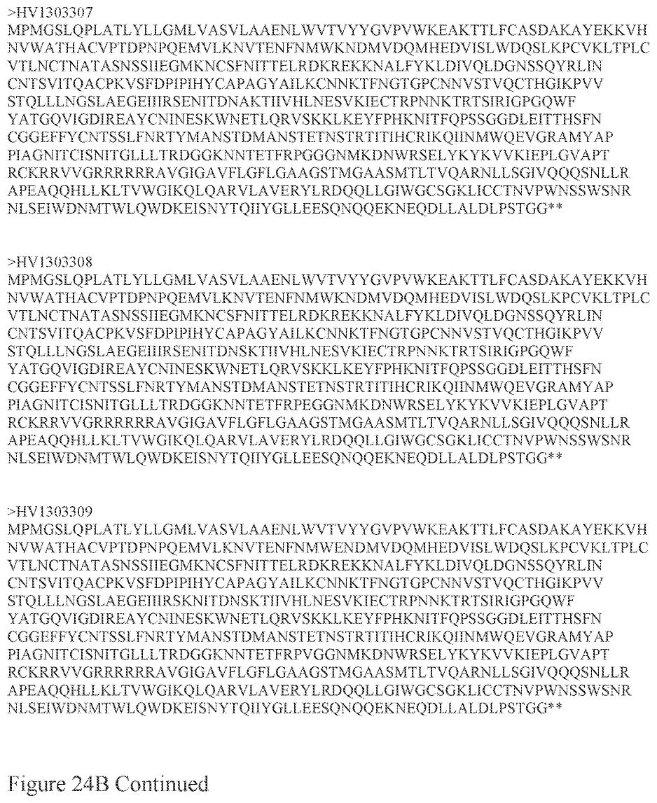

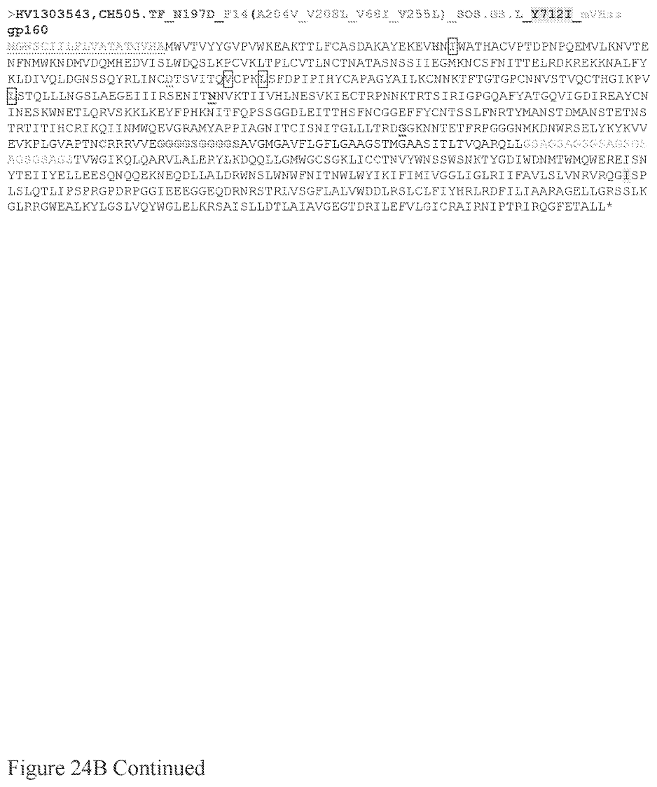

Non-limiting embodiments of envelope designs for use in sortase A reaction are shown in FIG. 24 B-D of U.S. patent Ser. No. 10/968,255, incorporated by reference in its entirety.

Non-limiting embodiments of sortase linkers could be used so long as their position allows multimerization of the envelopes. In a non-limiting embodiment, a C-terminal tag is LPXTG, where X signifies any amino acid but most commonly Ala, Ser, Glu, or a N-terminal pentaglycine repeat tag is added to the envelope trimer gene. In a non-limiting embodiment, a C-terminal tag is LPXTGG, where X signifies any amino acid but most commonly Ala, Ser, Glu.

For development as a vaccine immunogen, multimeric nanoparticles that comprise and/or display HIV envelope protein or fragments on their surface have also been created.

The nanoparticle immunogens are composed of various forms of HIV-envelope protein, e.g. without limitation envelope trimer, and self-assembling protein, e.g. without limitation ferritin protein. Any suitable ferritin could be used in the immunogens of the invention. In non-limiting embodiments, the ferritin is derived from Helicobacter pylori. In non-limiting embodiments, the ferritin is insect ferritin. In non-limiting embodiments, each nanoparticle displays 24 copies of the spike protein on its surface.

Presenting multiple copies of antigens to B cells has been a longstanding approach to improving B cell receptor recognition and antigen uptake (See Batista et al. EMBO J. 2000 Feb. 15; 19(4): 513-520). The improved recognition of antigen is due to the avid interaction of multiple antigens with multiple B cell receptors on a single B cells, which results in clustering of B cells and stronger cell signaling. Furthermore, multimeric presentation improves antigen binding to mannose binding lectin which promotes antigen trafficking to B cell follicles. Self-assembling complexes comprising multiple copies of an antigen are one strategy of immunogen design approach for arraying multiple copies of an antigen for recognition by the B cell receptors on B cells (Kanekiyo, M., Wei, C. J., Yassine, H. M., McTamney, P. M., Boyington, J. C., Whittle, J. R., Rao, S. S., Kong, W. P., Wang, L., and Nabel, G. J. (2013). Self-assembling influenza nanoparticle vaccines elicit broadly neutralizing H1N1 antibodies. Nature 499, 102-106; Ueda, G., Antanasijevic, A., Fallas, J. A., Sheffler, W., Copps, J., Ellis, D., Hutchinson, G. B., Moyer, A., Yasmeen, A., Tsybovsky, Y., et al. (2020), Tailored design of protein nanoparticle scaffolds for multivalent presentation of viral glycoprotein antigens. Elife).

In some instances, the gene of an antigen can be fused via a linker/spacer to a gene of a protein which could self-assemble. Upon translation, a fusion protein is made that can self-assemble into a multimeric complex—also referred to as a nanoparticle displaying multiple copies of the antigen. In other instances, the protein antigen could be conjugated to the self-assembling protein via an enzymatic reaction, thereby forming a nanoparticle displaying multiple copies of the antigen. Non-limiting embodiments of enzymatic conjugation include without limitation sortase mediated conjugation. In some embodiments, linkers for use in any of the designs of the invention could be 2-50 amino acids long, e.g. 2, 3, 4, 5, 6, 7, 8, 9, 10, 11, 12, 13, 14, 15, 16, 17, 18, 19, 20, 25, 30, 35, 40, 45, or 50 amino acids long. In certain embodiments, these linkers comprise glycine and serine amino acid in any suitable combination, and/or repeating units of combinations of glycine, serine and/or alanine.

Ferritin is a well-known protein that self-assembles into a hollow particle composed of repeating subunits. In some species ferritin nanoparticles are composed of 24 copies of a single subunit, whereas in other species it is composed of 12 copies each of two subunits.

Table 1 lists non-limiting embodiments of CH505 M5G458Y N197D designs. Envelopes with gp160 in the name are gp160 designs. The rest of the envelopes are gp140. Envelopes designed to form a multimeric complex, e.g. a ferritin based nanoparticle, have “Ferritin” in their name. Throughout this patent application CH505 TF N279K, which is CH505 TF envelope sequence comprising N279K substitution is interchangeably referred also as CH505 M5 and/or CH505 TF M5(N279K).

TABLE 1

| Plasmid ID | Protein ID | FIG. |
|---|---|---|
| HV1301288_{—} | CH505M5chim.6R.SOSIP.664v4.1_G458Y_N197D | 21A; 21B |
| G458Y_N197D | | |
| HV1302915 | CH505M5_G458Y_N197D_H66A_A582T_L587A_Y712I_{—} | 22A; 23 |
| | mVHss gp160 | |
| HV1302916 | CH505M5_G458Y_N197D_F14(A204V_V208L_V68I_{—} | 22B; 23 |
| | V255L)_Y712I_mVHss gp160 | |
| HV1302917 | CH505M5_G458Y_N197D_F14(A204V_V208_V68I_{—} | 22C; 23 |
| | V255L)_SOSL_Y712I_mVHss gp160 | |
| HV1302918 | CH505M5_G458Y_N197D_F14(A204V_V208L_V68I_{—} | 22D; 23 |
| | V255L)_SOS.GS.L_Y712I_mVHss gp160 | |
| HV1302919 | CH505M5_G458Y_N197D_H66A_A582T_L587A_{—} | 22E; 23 |
| | SOS.GS.L_Y712I_mVHss gp160 | |
| HV1302920 | CH505M5_G458Y_N197D_F14(A204V_V208L_V68I_{—} | 22F; 23 |
| | V255L)_SOS.GS.L.M535_Y712I_mVHss gp160 | |
| HV1302921 | M5.G458Y.N197D.chSOSIP_F14_25lnGSERK_{—} | 22G; 23 |
| | VRCFerritin_mVHss | |
| HV1302922 | M5.G458Y.N197D.chSOSIP_F14_14lnGSERK_{—} | 22H; 23 |
| | VRCFerritin_mVHss | |
| HV1302923 | M5.G458Y.N197D.chSOSIP_F14_10lnQQ_{—} | 22I; 23 |
| | VRCFerritin_mVHss | |
| HV1302924 | M5.G458Y.N197D.SOS.GS.L.M535_F14_25lnGSERK_{—} | 22J; 23 |
| | VRCFerritin_mVHss | |
| HV1302925 | M5_G458Y_N197D_SOS.GS.L.M535.F14_14lnGSERK_{—} | 22K |
| | VRCFerritin_mVHss | |
| HV1302926 | M5_G458Y_N197D_SOS.GS.L.M535_F14_10lnQQ_{—} | 22L |
| | VRCFerritin_mVHss | |
| HV1302927 | M5.G458Y.N197D.chSOSIP_F14.2Gly_mVHss | 22M |
| HV1302928 | M5.G458Y.N197D.SOS.GS.L.M535_F14.2Gly_mVHss | 22N |
| HV1302929 | M5.G458Y.N197D.chSOSIP_F14.3Gly_mVHss | 22O |
| HV1302930 | M5.G458Y.N197D.SOS.GS.L.M535_F14.3Gly_mVHss | 22P |
| HV1302931 | CH505.TF_H66A_A582T_L587A_Y712I_mVHss | 22Q; 23 |
| | gp160 | |
| HV1302932 | CH505.TF_F14(A204V_V208L_V68I_V255L)_{—} | 22R |
| | Y712I_mVHss gp160 | |
| HV1302933 | CH505.TF_F14(A204V_V208_V68I_V255L)_SOSL_{—} | 22S |
| | Y712I_mVHss gp160 | |
| HV1302934 | CH505.TF_F14(A204V_V208L_V68I_V255L)_{—} | 22T; 23 |
| | SOS.GS.L_Y712I_mVHss gp160 | |
| HV1302935 | CH505.TF_H66A_A582T_L587A_SOS.GS.L_Y712I_{—} | 22U |
| | mVHss gp160 | |
| HV1302936 | CH505.TF_F14(A204V_V208L_V68I_V255L)_{—} | 22V |
| | SOS.GS.L.M535_Y712I_mVHss gp160 | |
| HV1302937 | CH505.TF.chSOSIP_F14_25lnGSERK_VRCFerritin_{—} | 22W |
| | mVHss | |
| HV1302938 | CH505.TF.chSOSIP_F14_14lnGSERK_VRCFerritin_{—} | 22X |
| | mVHss | |
| HV1302939 | CH505.TF.chSOSIP_F14_10lnQQ_VRCFerritin_{—} | 22Y |
| | mVHss | |
| HV1302940 | CH505.TF.SOS.GS.L.M535_F14_25lnGSERK_{—} | 22Z |
| | VRCFerritin_mVHss | |
| HV1302941 | CH505.TF_SOS.GS.L.M535.F14_14lnGSERK_{—} | 22AA |
| | VRCFerritin_mVHss | |
| HV1302942 | CH505.TF_SOS.GS.L.M535_F14_10lnQQ_{—} | 22BB |
| | VRCFerritin_mVHss | |
| HV1302943 | CH505.TF.chSOSIP_F14.2Gly_mVHss | 22CC |
| HV1302944 | CH505.TF.SOS.GS.L.M535_F14.2Gly_mVHss | 22DD |
| HV1302945 | CH505.TF.chSOSIP_F14.3Gly_mVHss | 22EE |
| HV1302946 | CH505.TF.SOS.GS.L.M535_F14.3Gly_mVHss | 22FF |

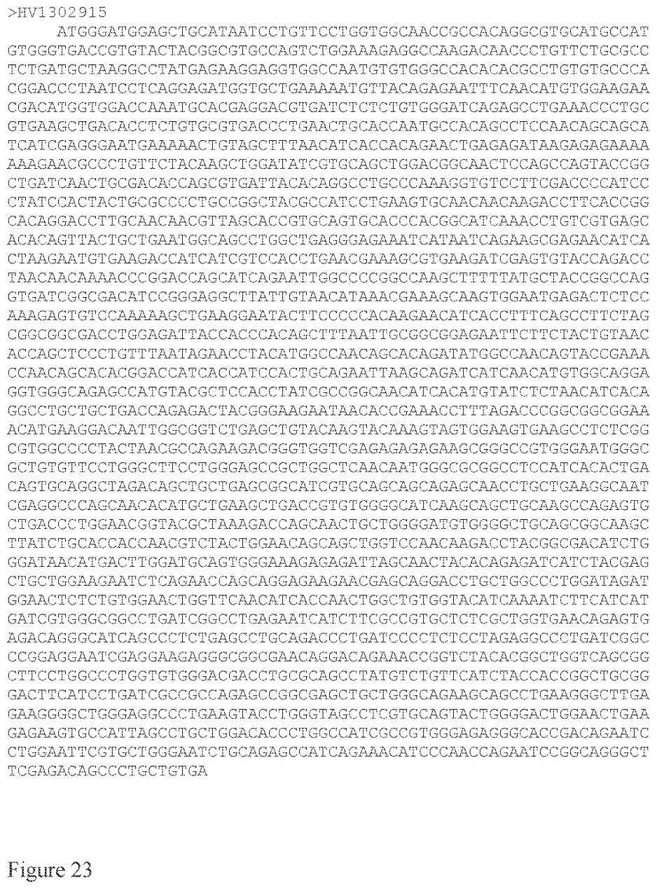

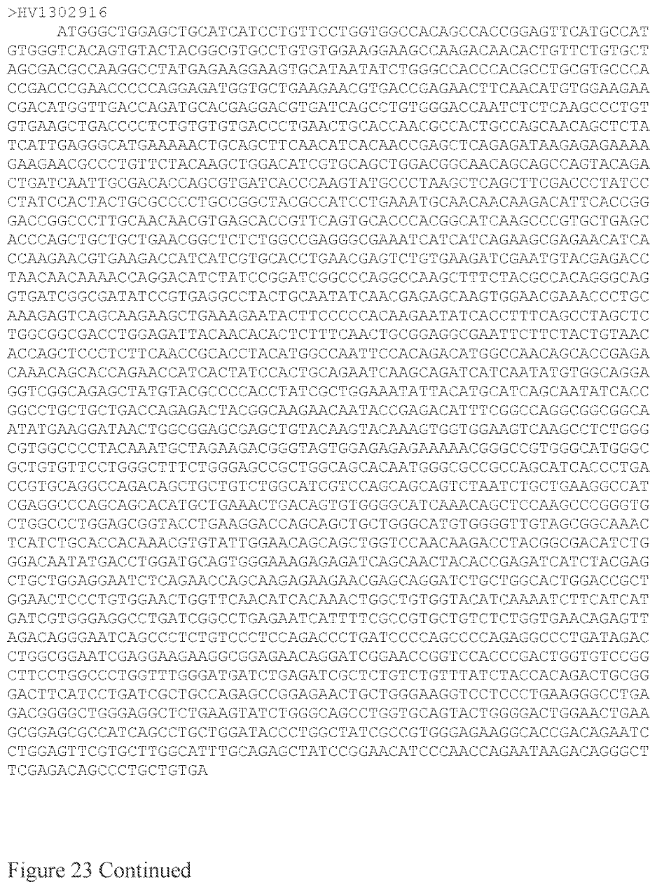

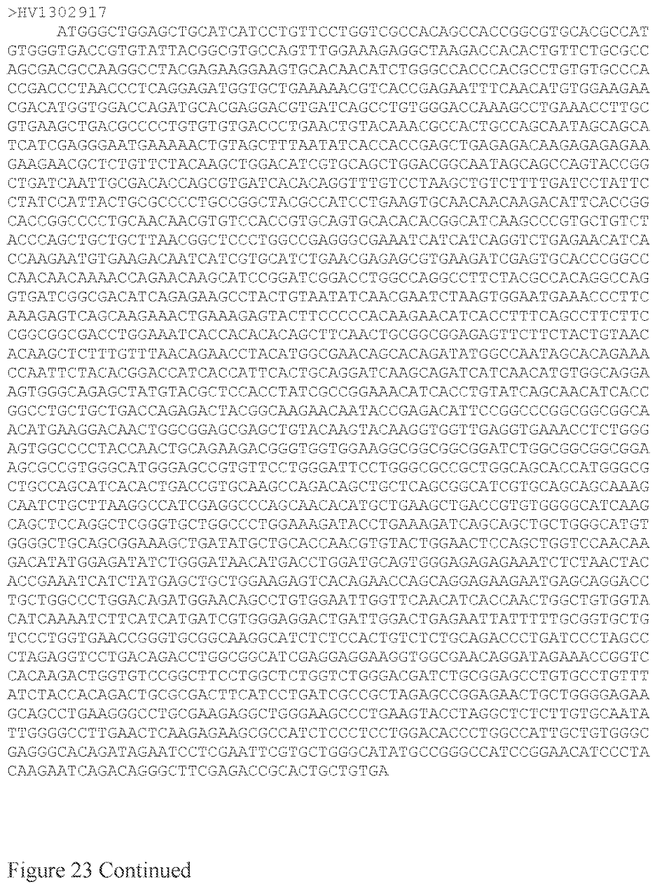

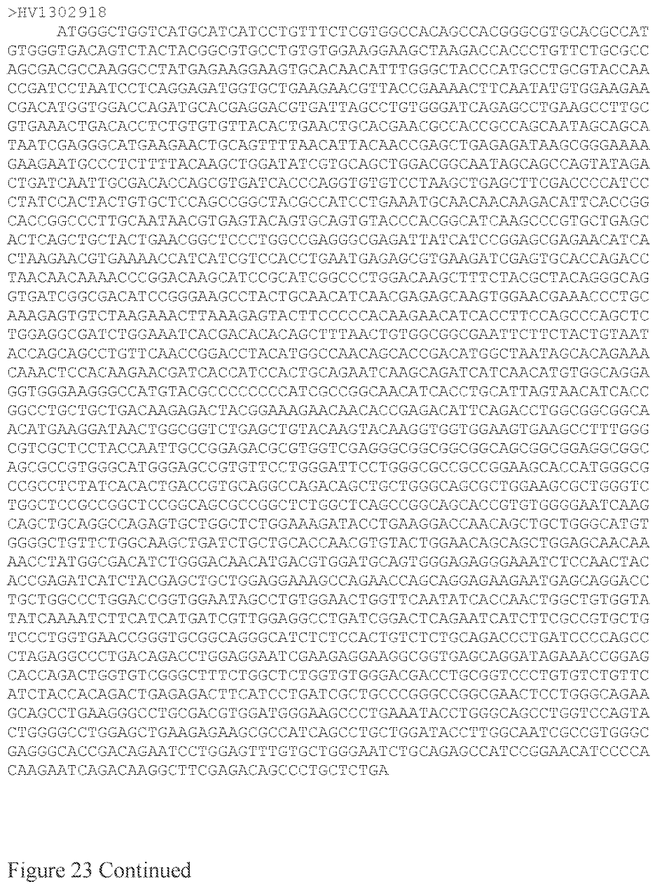

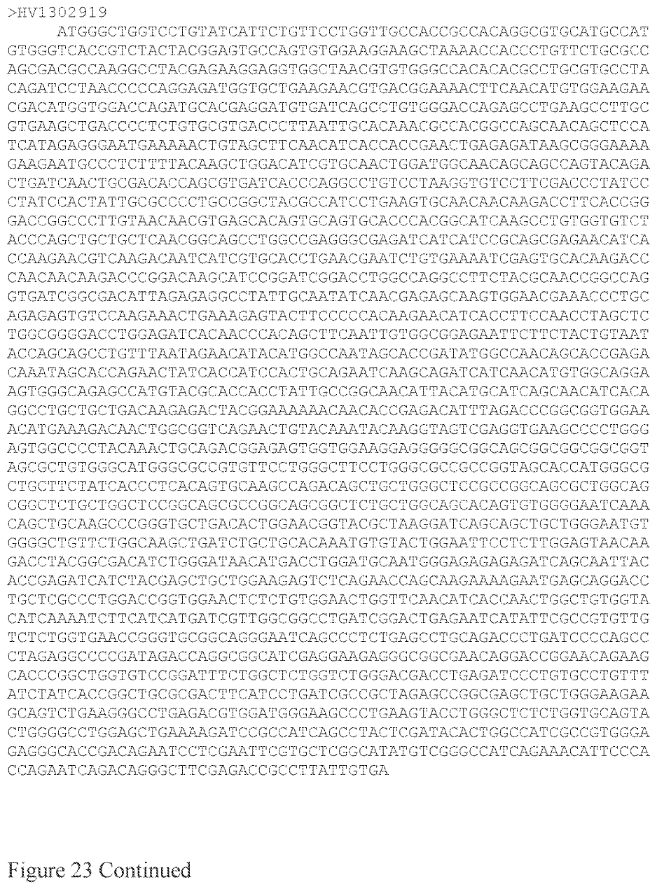

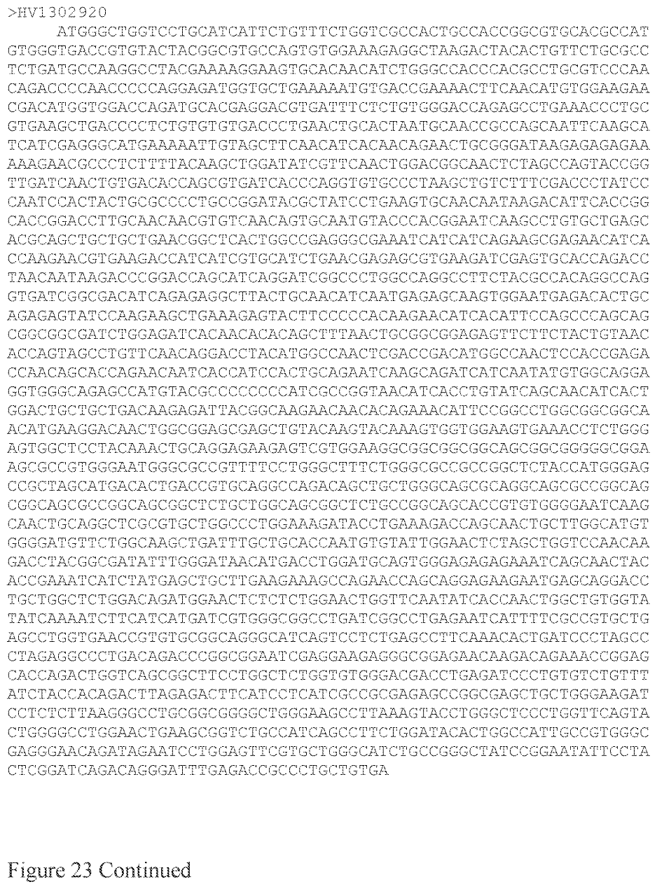

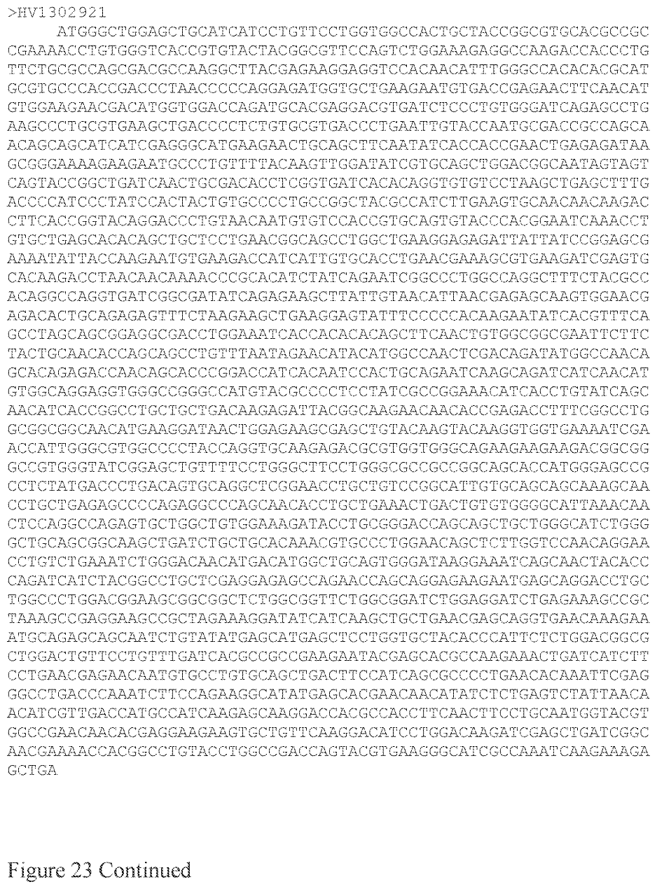

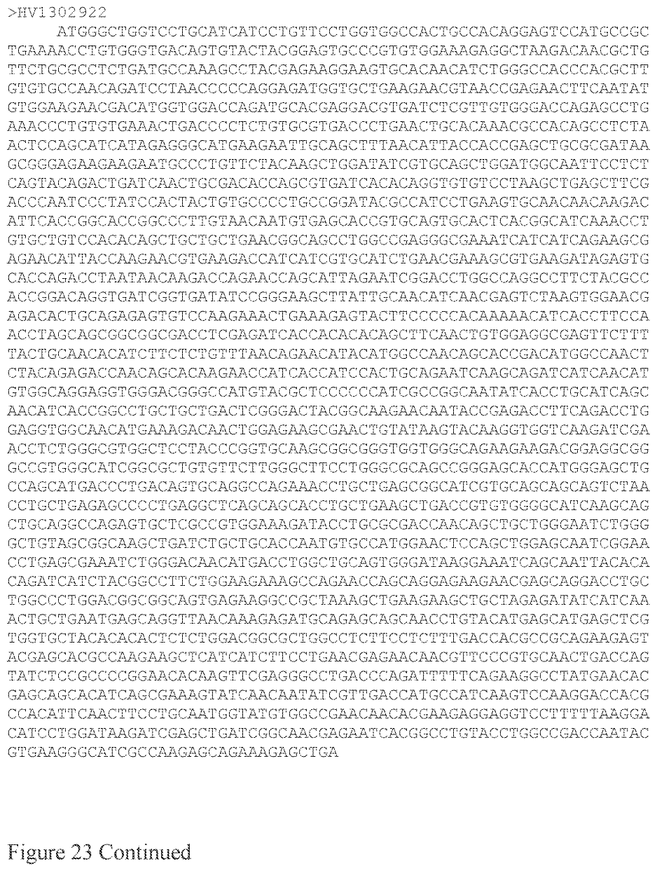

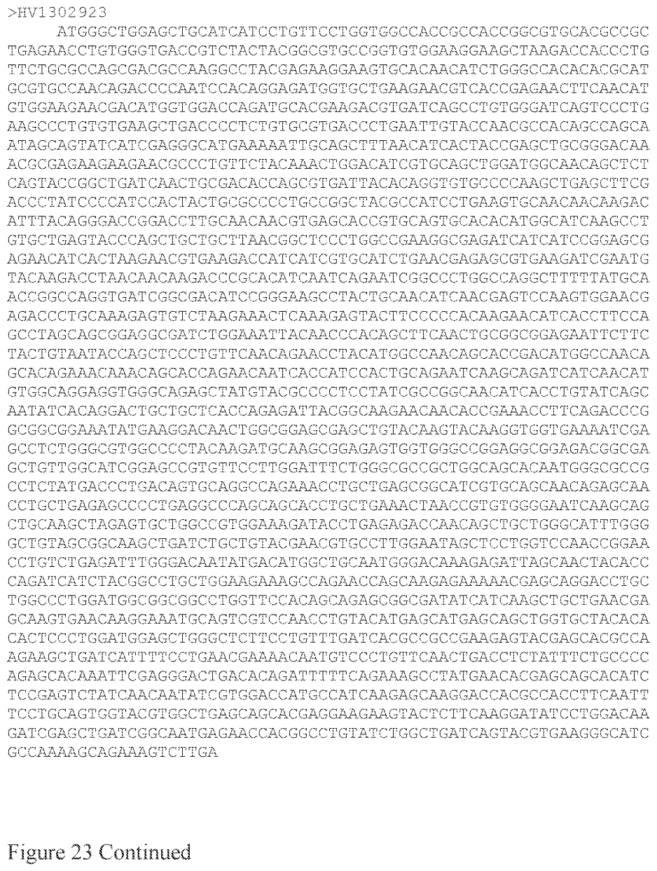

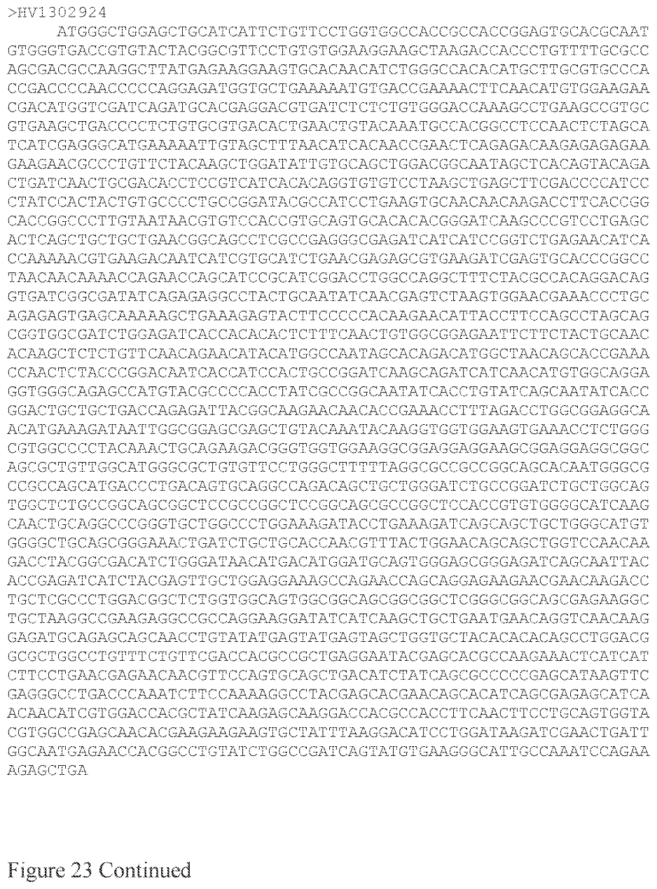

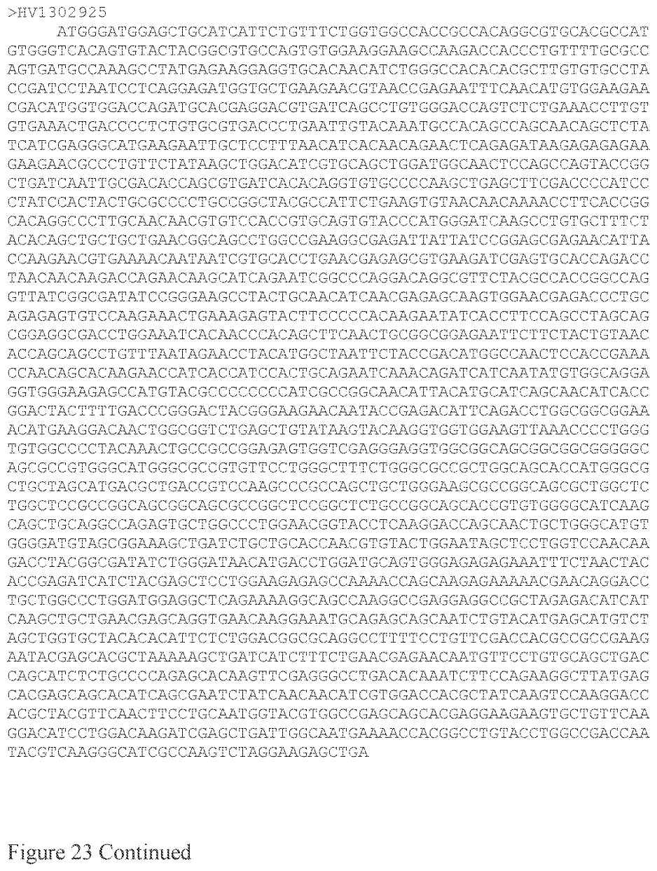

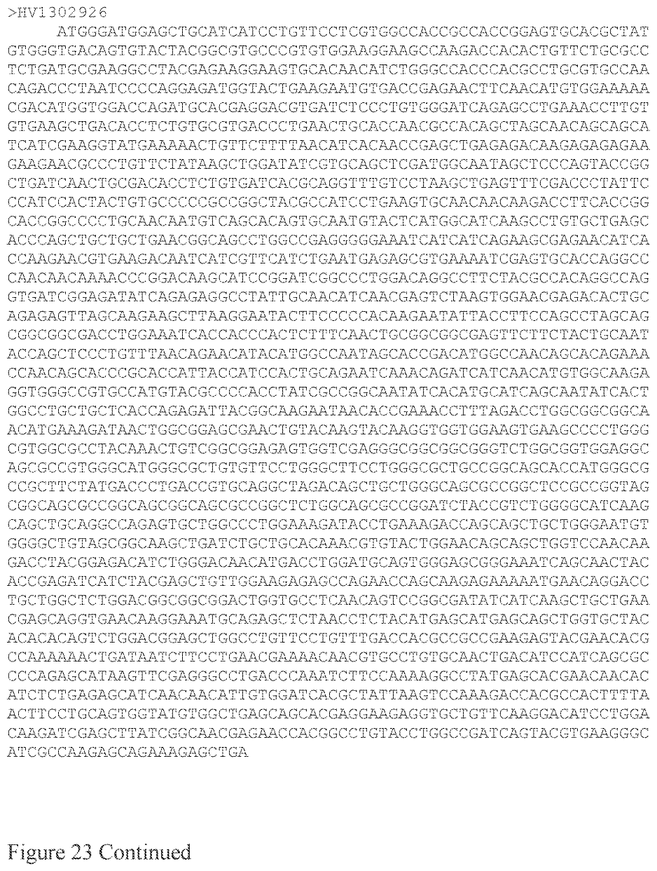

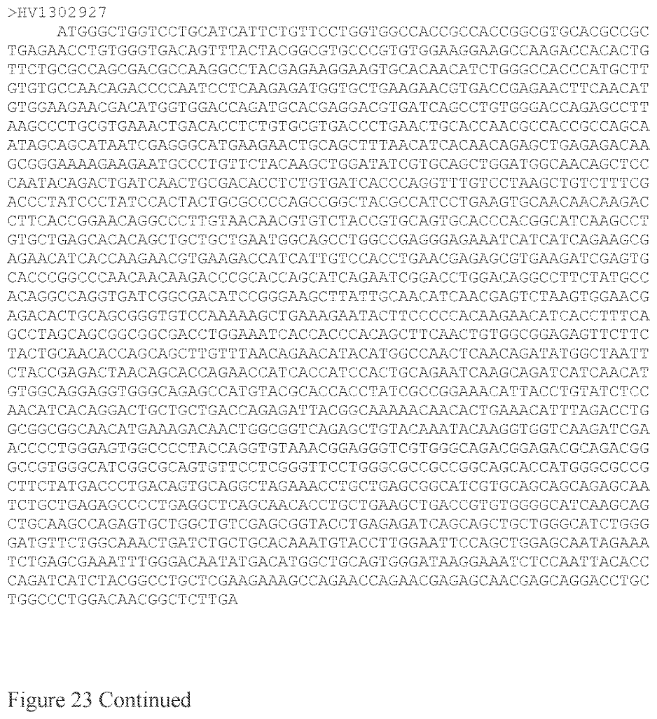

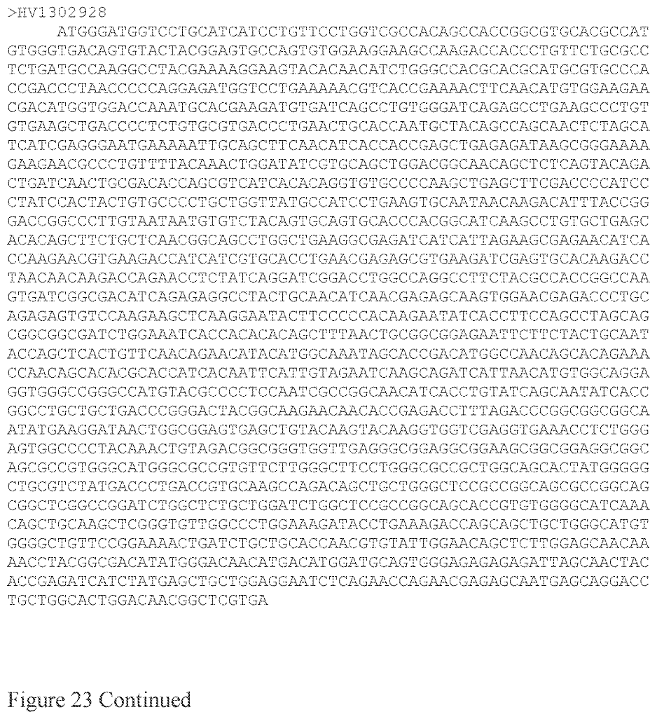

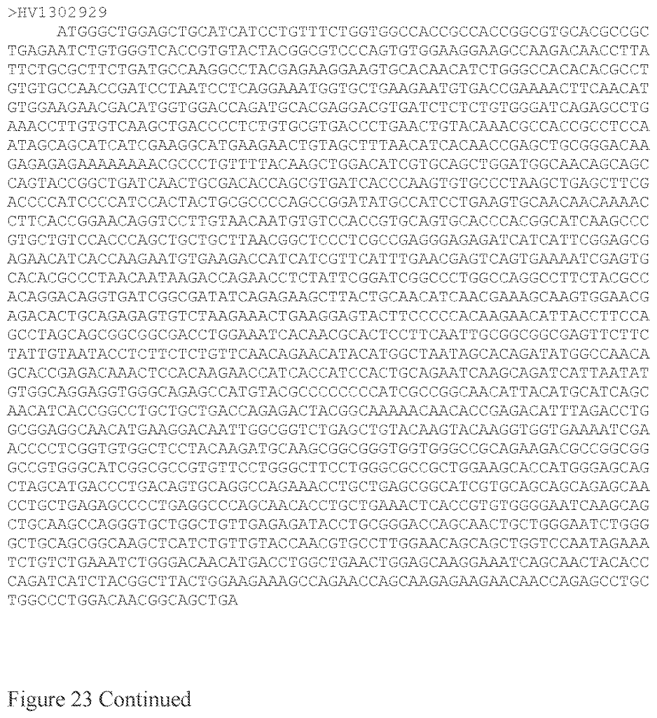

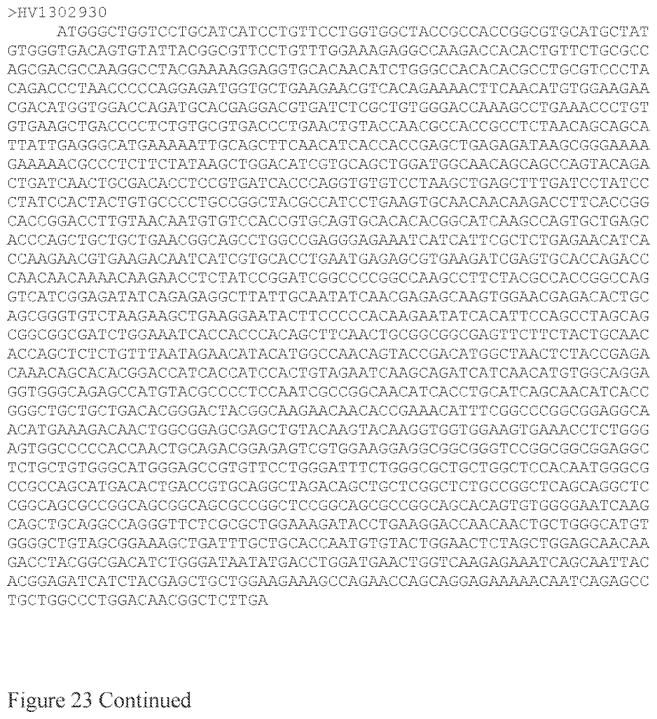

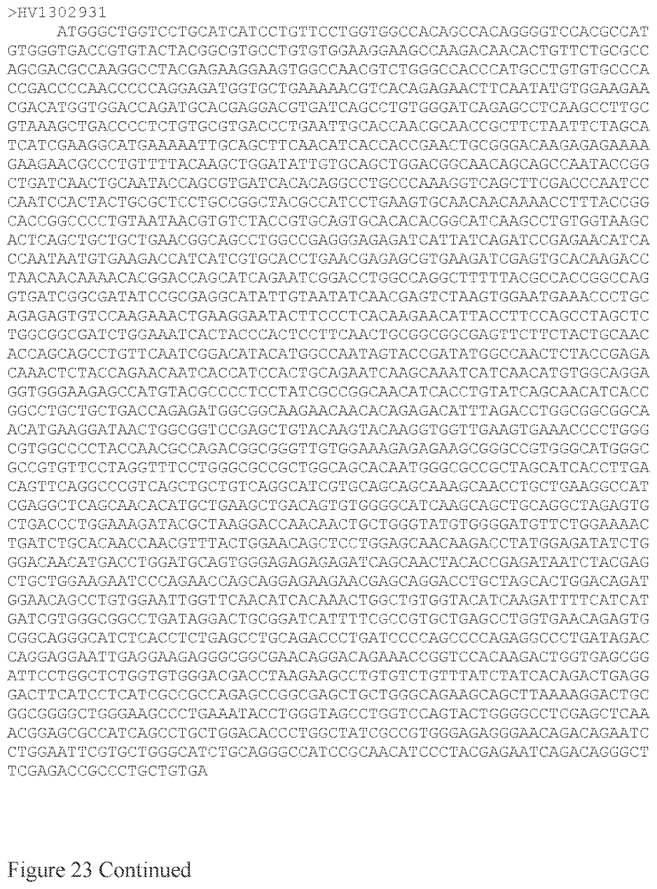

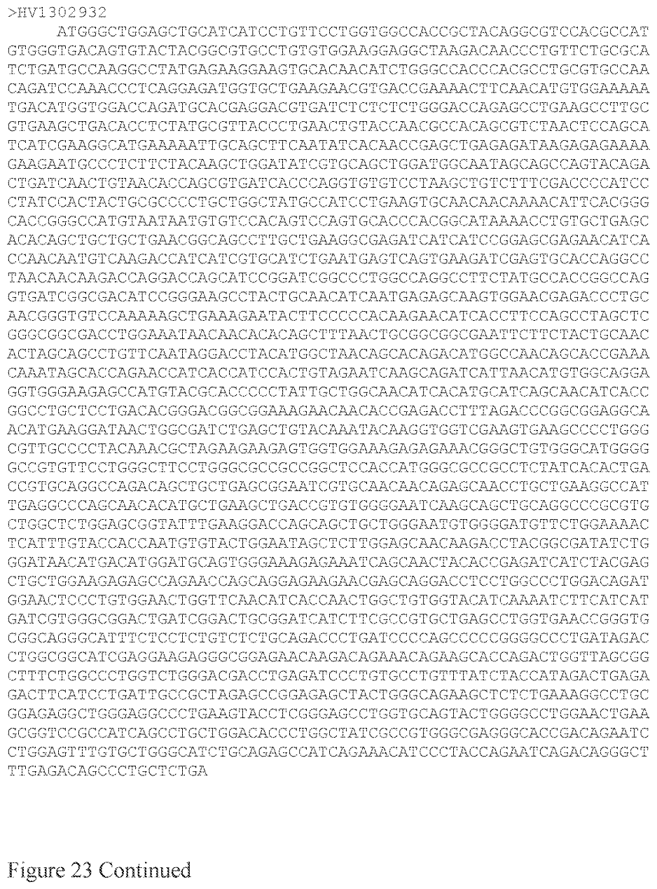

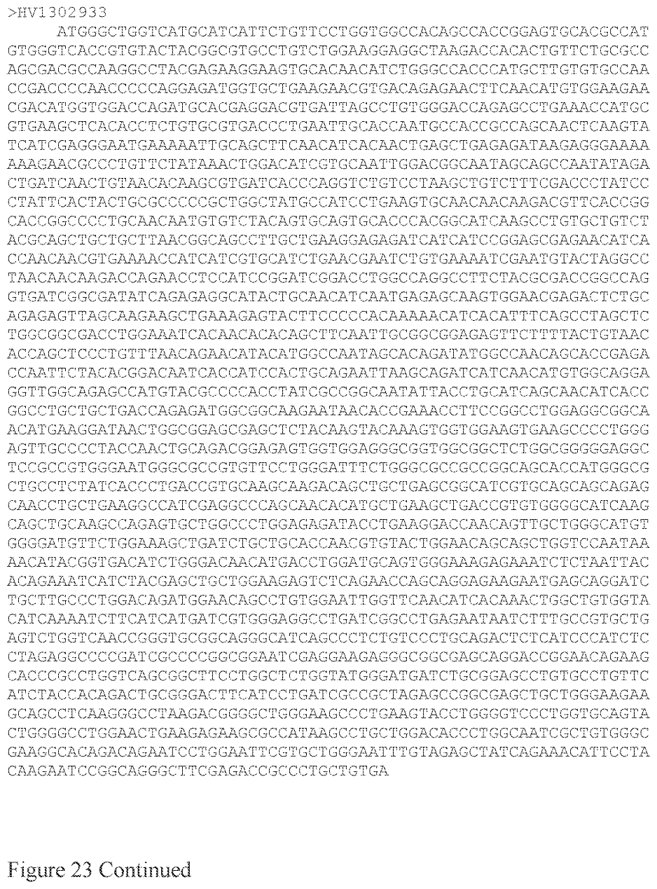

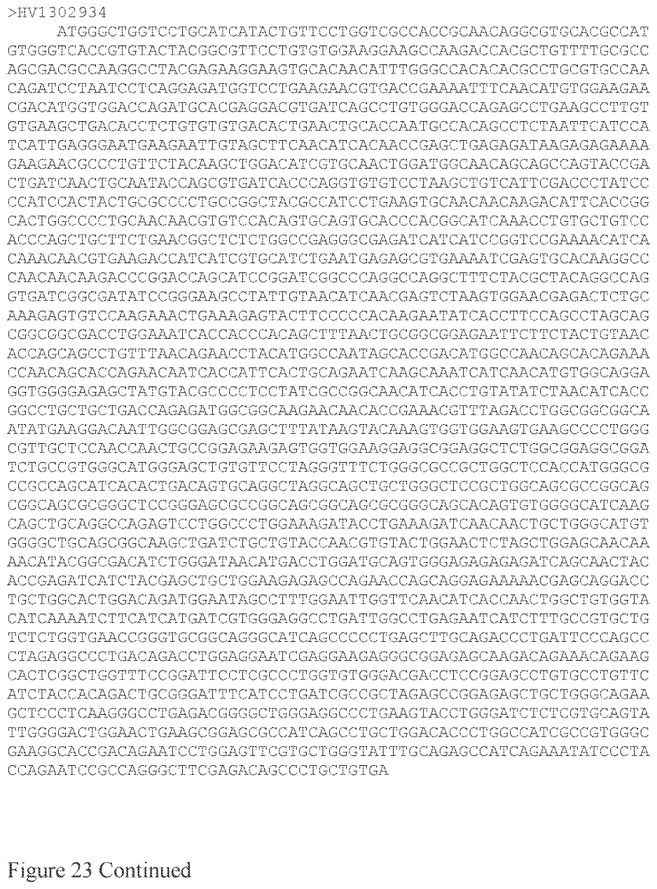

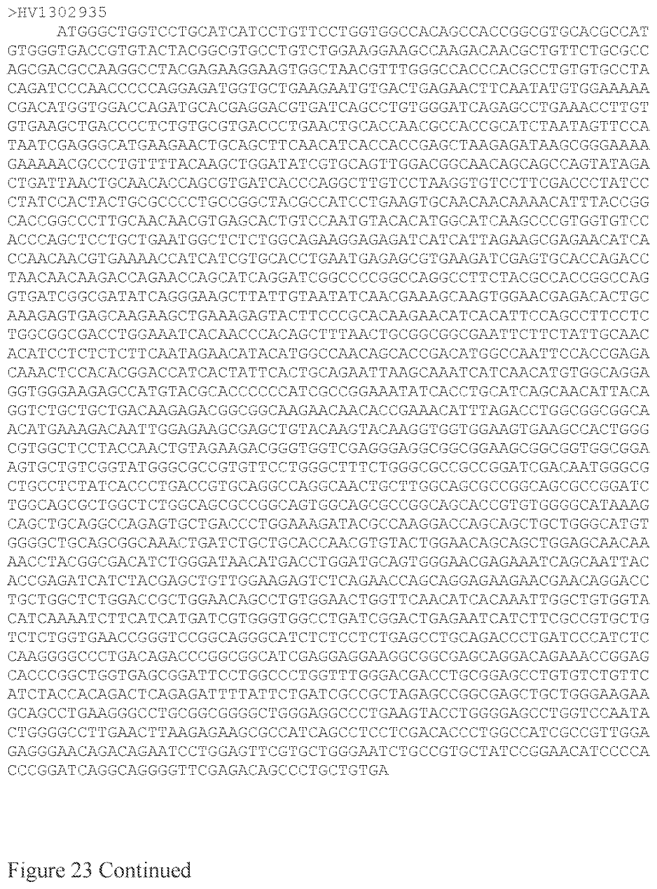

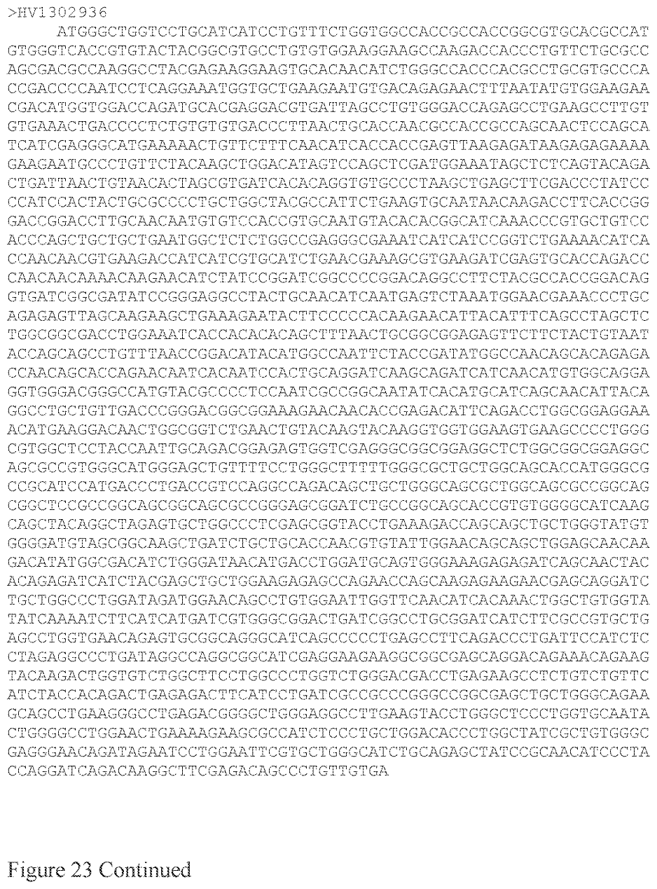

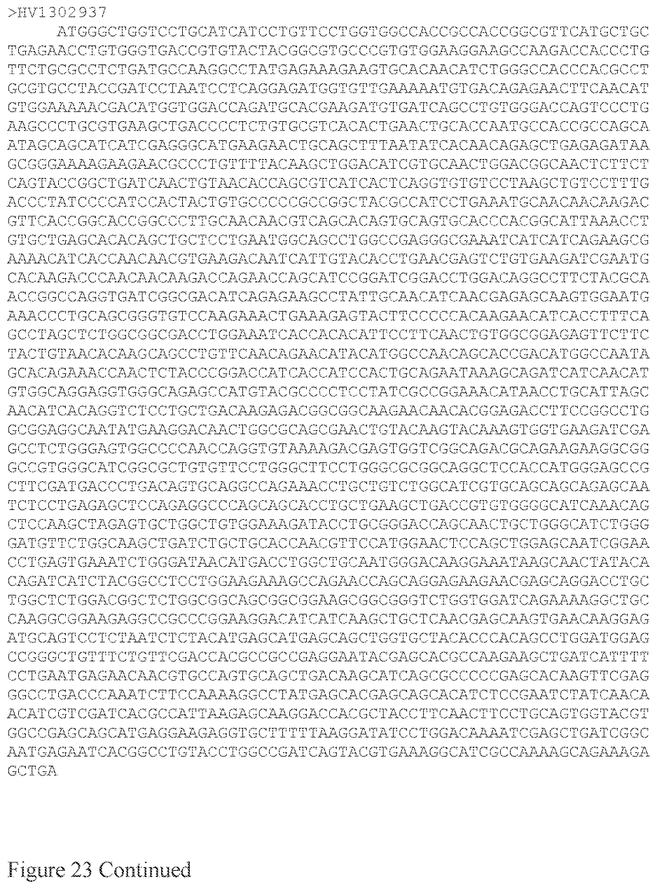

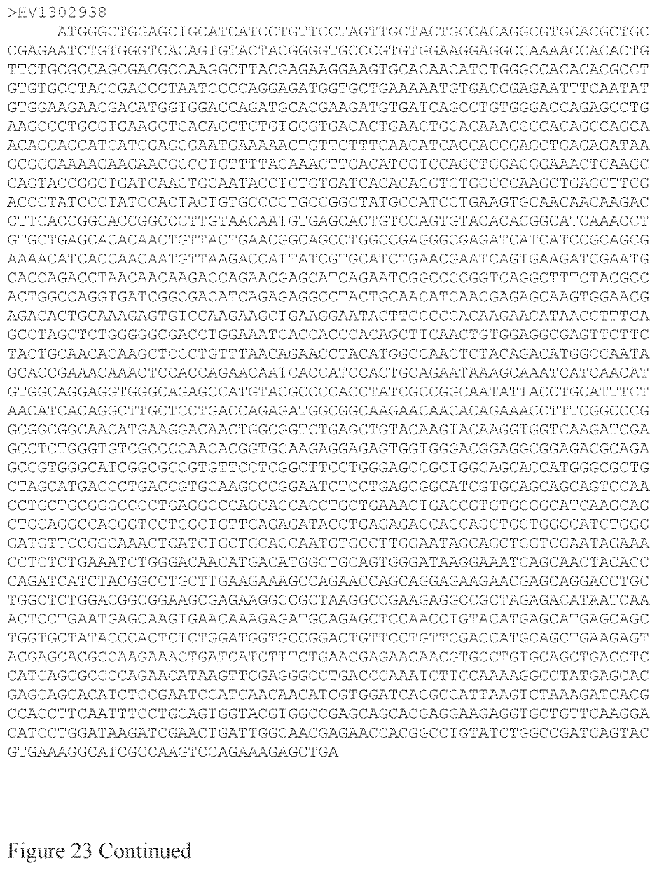

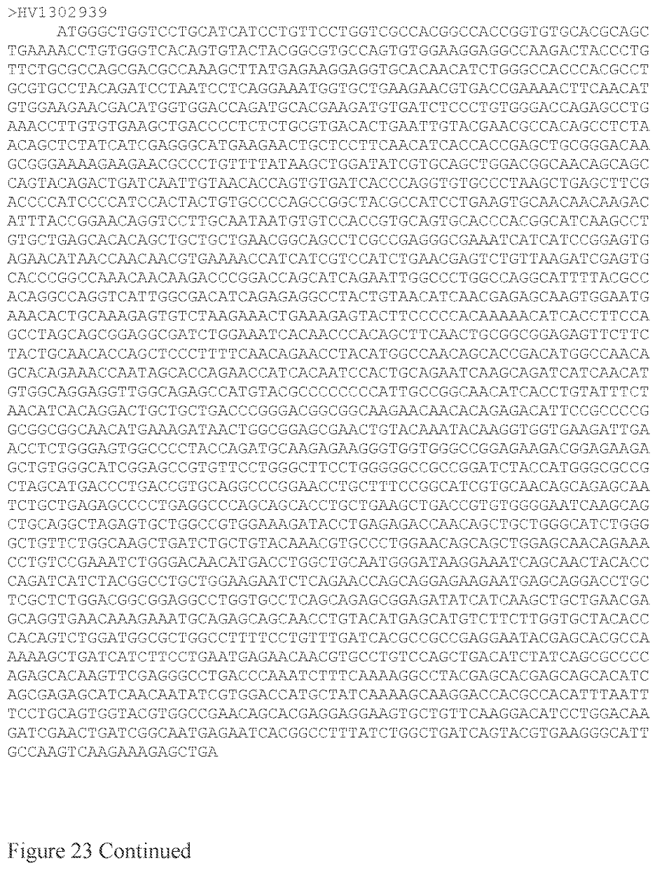

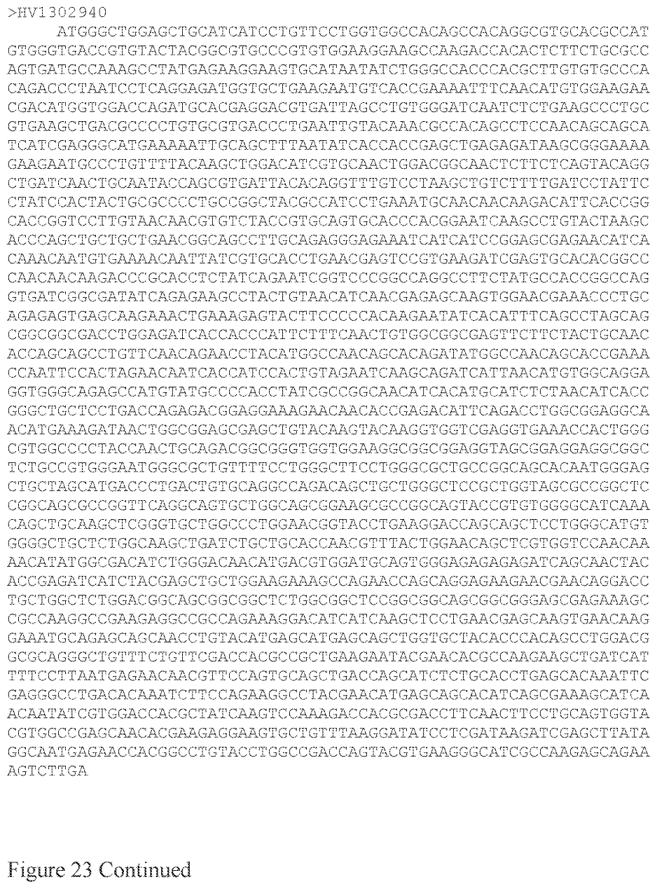

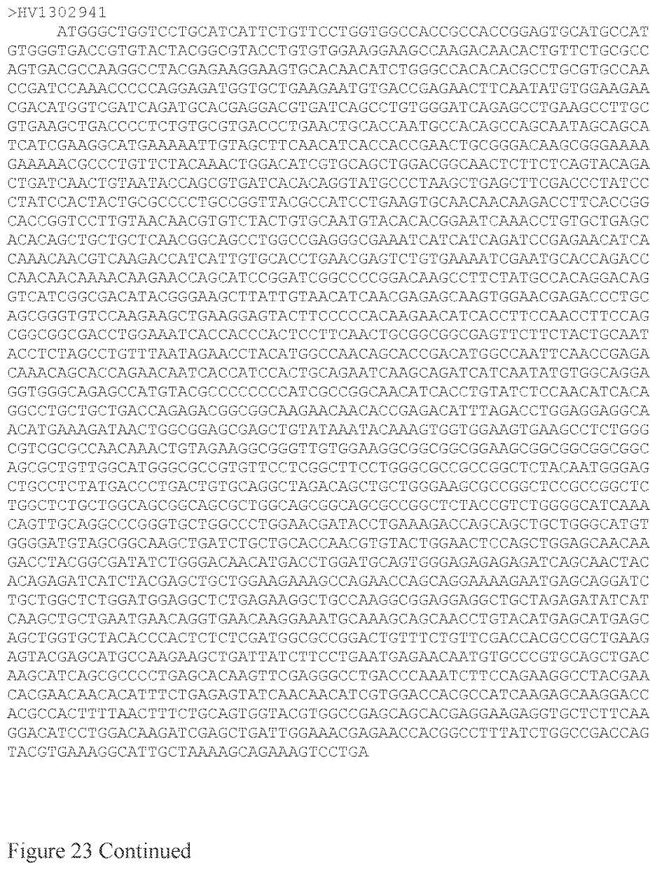

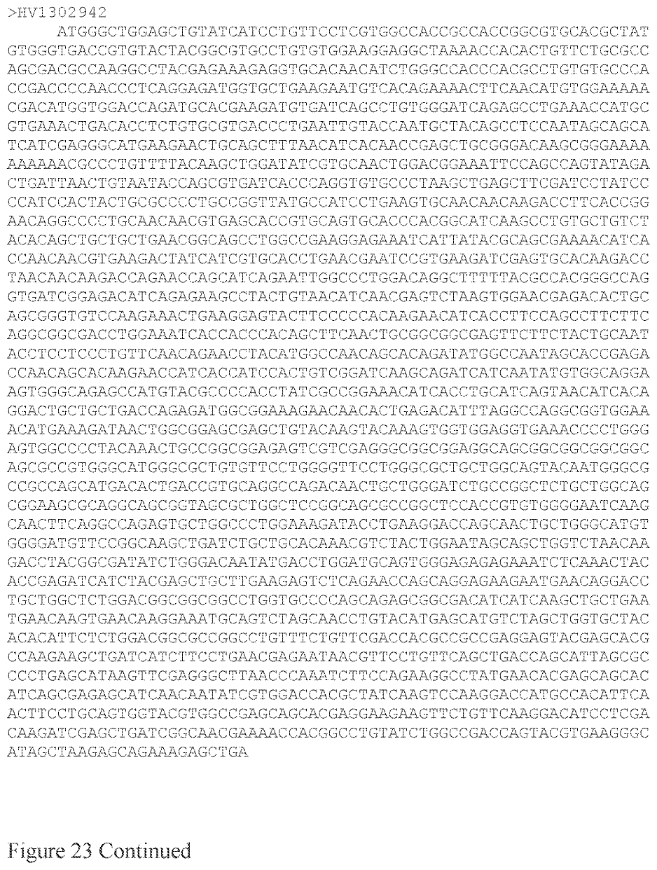

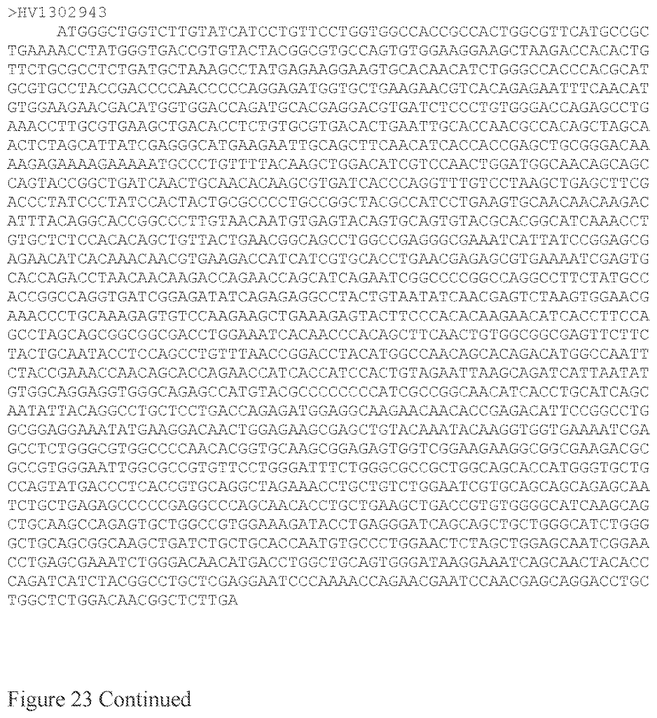

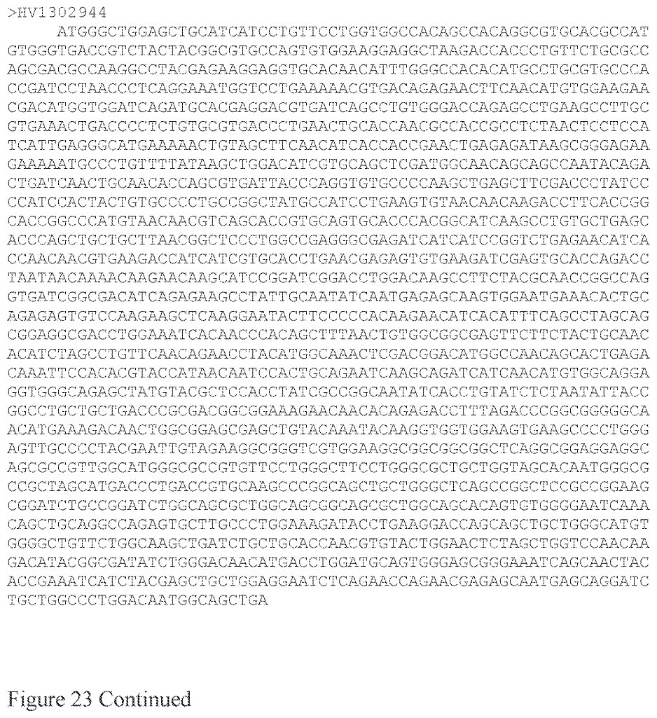

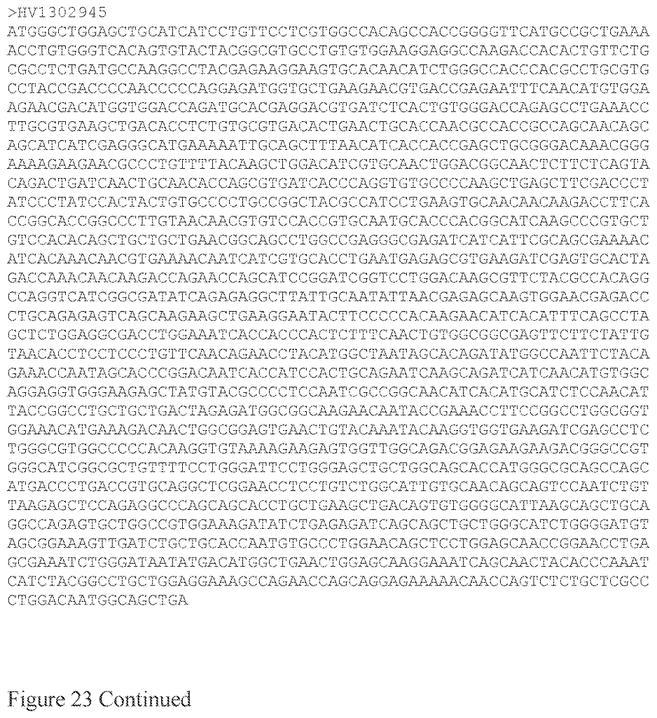

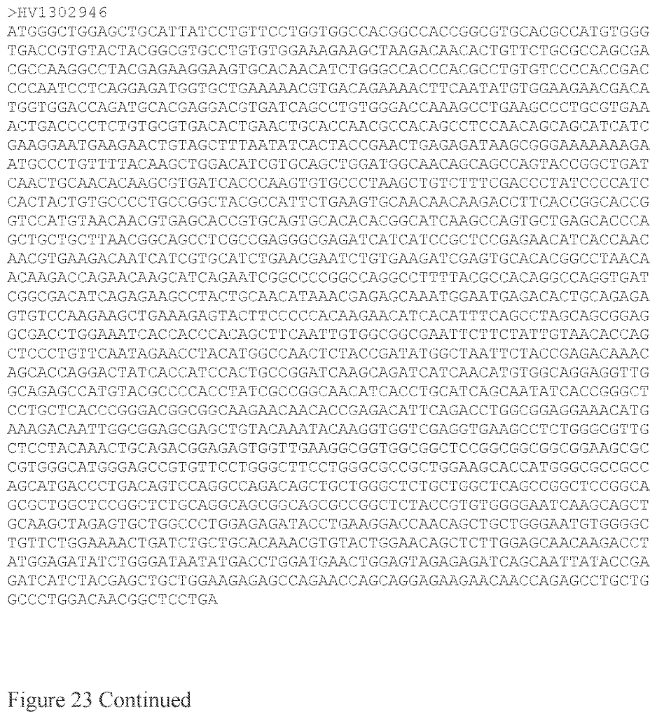

Non-limiting embodiments of sequences of the envelopes in Table 1 are described in FIGS. 22A through 22FF and FIG. 23. The designs in Table 1 could be used as the basis to design any suitable protomer, wherein in non-limiting embodiments the protomer can form stabilized trimer. Non-limiting designs of envelope protomers include SOSIP designs, designs comprising F14 mutations (See WO/2020/072169), and so forth.

The trimer could be incorporated in a nanoparticle, including without limitation any ferritin based nanoparticle.

Amino acid positions referred to herein refer to HXB2 numbering. See Korber, Bette, et al. “Numbering positions in HIV relative to HXB2CG.” Human retroviruses and AIDS 3 (1998): 102-111; see also B. Foley et al. “HIV Sequence Compendium 2018,” Published by Theoretical Biology and Biophysics Group, Los Alamos National Laboratory, NM, LA-UR 18-25673 accessible through www.hiv.lanl.gov, the content of each of which is hereby incorporated by reference in its entirety.

Non-limiting embodiments of linkers are listed in Table 2. In non-limiting embodiments the linker is LPXTGGGGGGSG or GSGLPXTGGGGG comprising linker. In some embodiments X is E. In non-limiting embodiments the linker is GSG(n) where n=1, 2, or 3. In non-limiting embodiments the linker is EKAAKAEEAAR, EKAAKAEEAARP, EKAAKAEEAARPP, GGSEKAAKAEEAAR, GGSEKAAKAEEAARP, or GGSEKAAKAEEAARPP. In certain embodiments, the linker is GGS(n), wherein n=1, 2, or 3. In certain embodiments, the linker is GGS(n)EKAAKAEEAAR(PP) the linker is EKAAKAEEAAR(PP)GGS(n) wherein n=1, 2, 3, 4, 5, or 6. In certain embodiments, the linker is GSG(n)EKAAKAEEAAR(K) or the linker is EKAAKAEEAAR(K)GSG(n) wherein n=1, 2, 3, 4, 5, or 6.

TABLE 2
Non-limiting embodiments of various linkers

| | |
|---|---|
| C-terminal Linker/sortase tag fused to | Linker between Env or portion thereof |
| envelope or portion thereof before sortase | and multimerizing protein (e.g. ferritin) |
| mediated conjugation | after sortase conjugation |
| These are short linkers to allow better | |
| access to the sortase A acceptor peptide. | |
| The goal is to extend the sortase A tag away | |
| from the RBD so that it can connect to the | |
| nanoparticle with sterically hitting the RBD | |
| beside it. | |
| LPXTG(GG) | LPXTGGGGGGSG (X could be E or S) |
| LPXTGG(G) | Or |
| LPXTGGG | LPXTGGGGG (X could be E or S) |
| (G) and (GG) are optional | |
| GSGLPXTG(GG) | GSGLPXTGGGGG (X could be E or S) |
| GSGLPXTGG(G) | |
| GSGLPXTGGG | |
| (G) and (GG) are optional | |
| Linkers in direct fusions between envelope | |
| or portion thereof and multimerizing protein | |
| (e.g. ferritin) | |
| [GGS](n) | Embodiment of a flexible linker |
| [GGS](n) | Where n = 1, 2,3, 4, 5, 6-10. |
| EKAAKAEEAAR | Embodiments of rigid linkers |
| EKAAKAEEAARP | |
| EKAAKAEEAARPP | |
| [GGS](n)EKAAKAEEAAR | Embodiments of flexible and rigid |
| [GGS](n)EKAAKAEEAARP | linkers |
| [GGS](n)EKAAKAEEAARPP | |
| [GGS](n)EKAAKAEEAAR | |
| [GGS](n)EKAAKAEEAARP | Where n = 1, 2,3, 4, 5, 6-10. |
| [GGS](n)EKAAKAEEAARPP | |
| [GGS](n)EKAAKAEEAARK | Embodiments of flexible and rigid |
| GSGGSGGSGGSGGSEKAAKAEEAARK | linkers |
| [GGS](n)EKAAKAEEAAR | Where n = 1, 2,3, 4, 5, 6-10. |
| GSGGSGGSGGSGGSEKAAKAEEAAR | |

Table 3 lists additional non-limiting embodiments of CH505 M5G458Y N197D designs. Envelopes with gp1160 in the name are gp1160 designs. The rest of the envelopes are gp140.

TABLE 3

| Protein ID | Protein Name | FIG. |
|---|---|---|
| HV1302458 | CH505.w24.e5.nCH.DS.SOSIP.CD5 | 24A; |
| | | 24B |
| HV1302459 | CH505.w24.e5.nCH.DS.SOSIP.UFO.CD5 | 24A; |
| | | 24B |
| HV1302460 | CH505.w24.e5.nCH.DS.SOSIP.UFO.TPA | 24A; |
| | | 24B |
| HV1302461 | CH505.w24.e5.nCH.DS.SOSIPv6.CD5 | 24A; |
| | | 24B |
| HV1302462 | CH505.w24.e5.nCH.DS.SOSIPv6.TPA | 24A; |
| | | 24B |
| HV1302660 | CH505.w24.e5.nCH.SOSIP.CD5ss | 24A; |
| | | 24B |
| HV1302661 | CH505.w24.e5.nCH.F14.SOSIP.CD5ss | 24A; |
| | | 24B |
| HV1302790 | CH505.w24.e5.nCH.F14.SOS.GS.I535M.V200A_CD5ss | 24A; |
| | | 24B |
| HV1302791 | CH505.w24.e5.nCH.F14.SOS.GS.I535M.V200A_mIgss | 24A; |
| | | 24B |
| HV1302792 | CH505.w24.e5.nCH.F14.SOS.UFO.I535M.V200A_CD5ss | 24A; |
| | | 24B |
| HV1302793 | CH505.w24.e5.nCH.F14.SOS.UFO.I535M.V200A_mIgss | 24A; |
| | | 24B |
| HV1303300 | CH505TFchim.6R.SOSIP.664.v4.1_w24Dloop_C_SORTAv3 | 24A; |
| | | 24B |
| HV1303301 | CH505TFchim.6R.SOSIP.664.v4.1_w24V5_C_SORTAv3 | 24A; |
| | | 24B |
| HV1303302 | CH505TFchim.6R.SOSIP.664.v4.1_w24Dloop- | 24A; |
| | V5_C_SORTAv3 | 24B |
| HV1303311 | CH505.TF.w24Dloop.nCH.F14.SOSIP.UFO.CD5ss_cSORTAv3 | 24A; |
| | | 24B |
| HV1303312 | CH505.TF.w24V5.nCH.F14.SOSIP.UFO.CD5ss_cSORTAv3 | 24A; |
| | | 24B |
| HV1303440 | CH505.w24.e5 F14.SOS.GSA.L.Y712I.I535M gp160_mVHss | 24A; |
| | | 24B |
| HV1303441 | CH505.w24.e5 F14.SOS.GSA.L.Y712I.I535M.A316W | 24A; |
| | gp160_mVHss | 24B |
| HV1303442 | CH505.w24.e5 F14.SOS.GSA.L.Y712I.I535M.A316W.VT8 | 24A; |
| | gp160_mVHss | 24B |
| HV1303443 | CH505.w24.e5 | 24A; |
| | F14.SOS.GSA.L.Y712I.I535M.A316W.S306L.R308L | 24B |
| | gp160_mVHss | |
| HV1302650 | AC10.29.nCH.SOSIP.UFO.TPAss | 24A; |
| | | 24B |
| HV1302651 | AC10.29.nCH.F14.SOSIP.UFO.TPAss | 24A; |
| | | 24B |
| HV1302652 | CH0505.C.w136.e.B23.nCH.SOSIP.UFO.TPAss( | 24A; |
| | | 24B |
| HV1302653 | CH0505.C.w136.e.B23.nCH.F14.SOSIP.UFO.TPAss | 24A; |
| | | 24B |
| HV1302654 | CH0505.C.w136.e.B23.nCH.SOSIP.CD5ss | 24A; |
| | | 24B |
| HV1302655 | CH0505.C.w136.e.B23.nCH.F14.SOSIP.CD5ss | 24A; |
| | | 24B |
| HV1302656 | CH0505.w176.e2.nCH.SOSIP.UFO.TPAss | 24A; |
| | | 24B |
| HV1302657 | CH0505.w176.e2.nCH.F14.SOSIP.UFO.TPAss | 24A; |
| | | 24B |
| HV1302658 | CH505.TF.nCH.SOSIP.UFO.CD5ss | 24A; |
| | | 24B |
| HV1302659 | CH505.TF.nCH.F14.SOSIP.UFO.CD5ss | 24A; |
| | | 24B |
| HV1302662 | CH505.w48.e28.nCH.SOSIP.CD5ss | 24A; |
| | | 24B |
| HV1302663 | CH505.w48.e28.nCH.F14.SOSIP.CD5ss | 24A; |
| | | 24B |
| HV1302664 | CH505.w96.A5.nCH.SOSIP.CD5ss | 24A; |
| | | 24B |
| HV1302665 | CH505.w96.A5.nCH.F14.SOSIP.CD5ss | 24A; |
| | | 24B |
| HV1302666 | SS1196.01.nCH.SOSIP.CD5ss | 24A; |
| | | 24B |
| HV1302667 | SS1196.01.nCH.F14.SOSIP.CD5ss | 24A; |
| | | 24B |
| HV1302668 | SS1196.01.nCH.SOSIP.UFO.CD5ss | 24A; |
| | | 24B |
| HV1302669 | SS1196.01.nCH.F14.SOSIP.UFO.CD5ss | 24A; |
| | | 24B |
| HV1303047 | CH505.w48.e28.nCH.F14.SOS.GS.I535M.V200A_mIgss | 24A; |
| | | 24B |
| HV1303048 | CH505.w96.A5.nCH.F14.SOS.GS.I535M.V200A_mIgss | 24A; |
| | | 24B |
| HV1303306 | CH505TFchim.SOSIPv4.1_SigMut1.T234N_N279D_V281G_{—} | 24A; |
| | cSORTAv3 | 24B |
| HV1303307 | CH505TFchim.SOSIPv4.1_SigMut2.T234N_N279D_V281A_{—} | 24A; |
| | cSORTAv3 | 24B |
| HV1303308 | CH505TFchim.SOSIPv4.1_SigMut3.T234N_N279D_V281S_{—} | 24A; |
| | G471E_cSORTAv3 | 24B |
| HV1303309 | CH505TFchim.SOSIPv4.1_SigMut4.K97E_T234N_E275K_{—} | 24A; |
| | N279D_V281S_G471V_cSORTAv3 | 24B |
| HV1303543 | CH505.TF_N197D_F14(A204V_V208L_V68I_V255L)_{—} | 24A; |
| | SOS.GS.L_Y712I_mVHss gp160 | 24B |

Non-limiting embodiments of sequences of the envelopes in Table 3 are described in FIG. 24. The designs in Table 3 could be used as the basis to design any suitable protomer, wherein in non-limiting embodiments the protomer can form stabilized trimer. Non-limiting designs of envelope protomers include SOSIP designs, designs comprising F14 mutations (See WO/2020/072169), and so forth.

The trimer could be incorporated in a nanoparticle, including without limitation any ferritin based nanoparticle.

Amino acid positions referred to herein refer to HXB2 numbering. See Korber, Bette, et al. “Numbering positions in HIV relative to HXB2CG.” Human retroviruses and AIDS 3 (1998): 102-111; see also B. Foley et al. “HIV Sequence Compendium 2018,” Published by Theoretical Biology and Biophysics Group, Los Alamos National Laboratory, NM, LA-UR 18-25673 accessible through www.hiv.lanl.gov, the content of each of which is hereby incorporated by reference in its entirety.

Any of the immunogens herein may be encoded by a nucleic acid. FIG. 21A, 23 or 24A provide non-limiting examples of nucleic acids encoding an immunogen. In certain embodiments, the nucleic acid may be a DNA, an RNA, or an mRNA.

Table 4 provides a list of non-limiting embodiments of CH505 M5G458Y N197D mRNA designs.

| Protein ID | Protein Name | FIG. |
|---|---|---|
| HV1302458 | CH505.w24.e5.nCH.DS.SOSIP.CD5 | 25 |
| HV1302459 | CH505.w24.e5.nCH.DS.SOSIP.UFO.CD5 | 25 |
| HV1302460 | CH505.w24.e5.nCH.DS.SOSIP.UFO.TPA | 25 |
| HV1302461 | CH505.w24.e5.nCH.DS.SOSIPv6.CD5 | 25 |
| HV1302462 | CH505.w24.e5.nCH.DS.SOSIPv6.TPA | 25 |
| HV1302660 | CH505.w24.e5.nCH.SOSIP.CD5ss | 25 |
| HV1302661 | CH505.w24.e5.nCH.F14.SOSIP.CD5ss | 25 |
| HV1302790 | CH505.w24.e5.nCH.F14.SOS.GS.I535M.V200A_CD5ss | 25 |
| HV1302791 | CH505.w24.e5.nCH.F14.SOS.GS.I535M.V200A_mIgss | 25 |
| HV1302792 | CH505.w24.e5.nCH.F14.SOS.UFO.I535M.V200A_CD5ss | 25 |
| HV1302793 | CH505.w24.e5.nCH.F14.SOS.UFO.1535M.V200A_mIgss | 25 |
| HV1303300 | CH505TFchim.6R.SOSIP.664.v4.1_w24Dloop_C_SORTAv3 | 25 |
| HV1303301 | CH505TFchim.6R.SOSIP.664.v4.1_w24V5_C_SORTAv3 | 25 |
| HV1303302 | CH505TFchim.6R.SOSIP.664.v4.1_w24Dloop-V5_C_SORTAv3 | 25 |
| HV1303311 | CH505.TF.w24Dloop.nCH.F14.SOSIP.UFO.CD5ss_cSORTAv3 | 25 |
| HV1303312 | CH505.TF.w24V5.nCH.F14.SOSIP.UFO.CD5ss_cSORTAv3 | 25 |
| HV1303440 | CH505.w24.e5 F14.SOS.GSA.L.Y712I.I535M gp160_mVHss | 25 |
| HV1303441 | CH505.w24.e5 F14.SOS.GSA.L.Y712I.I535M.A316W | 25 |
| | gp160_mVHss | |
| HV1303442 | CH505.w24.e5 F14.SOS.GSA.L.Y712I.I535M.A316W.VT8 | 25 |
| | gp160_mVHss | |
| HV1303443 | CH505.w24.e5 | 25 |
| | F14.SOS.GSA.L.Y712I.I535M.A316W.S306L.R308L | |
| | gp160_mVHss | |
| HV1302650 | AC10.29.nCH.SOSIP.UFO.TPAss | 25 |
| HV1302651 | AC10.29.nCH.F14.SOSIP.UFO.TPAss | 25 |
| HV1302652 | CH0505.C.w136.e.B23.nCH.SOSIP.UFO.TPAss( | 25 |
| HV1302653 | CH0505.C.w136.e.B23.nCH.F14.SOSIP.UFO.TPAss | 25 |
| HV1302654 | CH0505.C.w136.e.B23.nCH.SOSIP.CD5ss | 25 |
| HV1302655 | CH0505.C.w136.e.B23.nCH.F14.SOSIP.CD5ss | 25 |
| HV1302656 | CH0505.w176.e2.nCH.SOSIP.UFO.TPAss | 25 |
| HV1302657 | CH0505.w176.e2.nCH.F14.SOSIP.UFO.TPAss | 25 |
| HV1302658 | CH505.TF.nCH.SOSIP.UFO.CD5ss | 25 |
| HV1302659 | CH505.TF.nCH.F14.SOSIP.UFO.CD5ss | 25 |
| HV1302662 | CH505.w48.e28.nCH.SOSIP.CD5ss | 25 |
| HV1302663 | CH505.w48.e28.nCH.F14.SOSIP.CD5ss | 25 |
| HV1302664 | CH505.w96.A5.nCH.SOSIP.CD5ss | 25 |
| HV1302665 | CH505.w96.A5.nCH.F14.SOSIP.CD5ss | 25 |
| HV1302666 | SS1196.01.nCH.SOSIP.CD5ss | 25 |
| HV1302667 | SS1196.01.nCH.F14.SOSIP.CD5ss | 25 |
| HV1302668 | SS1196.01.nCH.SOSIP.UFO.CD5ss | 25 |
| HV1302669 | SS1196.01.nCH.F14.SOSIP.UFO.CD5ss | 25 |
| HV1303047 | CH505.w48.e28.nCH.F14.SOS.GS.I535M.V200A_mIgss | 25 |
| HV1303048 | CH505.w96.A5.nCH.F14.SOS.GS.I535M.V200A_mIgss | 25 |
| HV1303306 | CH505TFchim.SOSIPv4.1_SigMut1.T234N_N279D_V281G_{—} | 25 |
| | cSORTAv3 | |
| HV1303307 | CH505TFchim.SOSIPv4.1_SigMut2.T234N_N279D_V281A_{—} | 25 |
| | cSORTAv3 | |
| HV1303308 | CH505TFchim.SOSIPv4.1_SigMut3.T234N_N279D_V281S_{—} | 25 |
| | G471E_cSORTAv3 | |
| HV1303309 | CH505TFchim.SOSIPv4.1_SigMut4.K97E_T234N_E275K_N279D_{—} | 25 |
| | V281S_G471V_cSORTAv3 | |
| HV1303543 | CH505.TF_N197D_F14 (A204V_V208L_V68I_V255L)_{—} | 25 |
| | SOS.GS.L_Y712I_mVHss gp160 | |

In some embodiments, the mRNA provided herein in Table 4 and FIG. 25 are cloned between a 5′UTR and 3′UTR. Thus, in some embodiments, the nucleotide sequence of the inventions comprises a 5′UTR sequence, nucleotide sequence provided herein in Table 4 and FIG. 25, and a 3′UTR sequence. In some embodiments, the 5′UTR, including a Kozak sequence comprises the nucleic acid sequence in Table 5. In some embodiments, the 3′UTR, including a poly A sequence comprises the nucleic acid sequence in Table 5.

TABLE 5

| Name | Sequence |
|---|---|
| 5′UTR with | AGCATAAAAGTCTCAACACAACATATACAAAACAAACGAATCTC |
| Kozak | AAGCAATCAAGCATTCTACTTCTATTGCAGCAATTTAAATCATTTC |
| | TTTTAAAGCAAAAGCAATTTTCTGAAAATTTTCACCATTTACGAA |
| | CGATAGCGCTGCCACC |
| 3′UTR with | ACTAGTAGTGACTGACTAGGATCTGGTTACCACTAAACCAGCCTC |
| Poly A | AAGAACACCCGAATGGAGTCTCTAAGCTACATAATACCAACTTAC |
| (underlined) | ACTTACAAAATGTTGTCCCCCAAAATGTAGCCATTCGTATCTGCT |
| | CCTAATAAAAAGAAAGTTTCTTCACATTCTAAAAAAAAAAAAAA |
| | AAAAAAAAAAAAAAAAAAAAAAAAAAAAAAAAAAAAAAAAAA |
| | AAAAAAAAAAAAAAAAAAAAAAAAAAAAAAAAAAAAAAAAAA |
| | AAA |

The 3′UTR sequence above has a poly A tail length of 101 nucleotides. However, it should be understood that mRNA sequences can comprise different lengths of poly A tail. For example, in some embodiments the poly A tail is about 85 to about 200 nucleotides long. For example, in some embodiments the poly A tail is 85 to 200 nucleotides long. In some embodiments the poly A tail is about 85 to about 110 nucleotides long. In some embodiments the poly A tail is 85 to 110 nucleotides long. In some embodiments the poly A tail is about 90 to about 110 nucleotides long. In some embodiments the poly A tail is 90 to 110 nucleotides long.

The invention is described in the following non-limiting examples.

### EXAMPLES

### Example 1: CH505 M5G458Y

LaBranche C et al. have reported that CH505 M5 envelope and/or virus comprising G458Y mutation shows improved neutralization of CH235 UCA. See PLoS Pathog. 2019 Sep. 17; 15(9):e1008026. doi: 10.1371/journal.ppat.1008026, incorporated by reference in its entirety.

### Example 2: CH505 M5G458Y N197D Designs

The goal of HIV-1 vaccine design is to elicit broadly neutralizing antibodies that can protect against infection. Vaccine immunogens that can specifically engage the B cell receptors of naïve B cells prior to affinity maturation are needed in order to elicit such antibodies. The problem is that most B cell receptors composed of unmutated or germline broadly neutralizing antibody precursors exhibit highly selective reactivity with HIV-1 envelope. To circumvent this problem immunogens must be designed that bind to the unmutated precursor antibodies for broadly neutralizing antibody lineages.

We previously isolated the CH235 broadly neutralizing antibody lineage from the HIV-1 infected individual called CH505. The transmitted founder virus of CH505 was cloned to test reactivity with the unmutated precursor of the CH235 broadly neutralizing antibody lineage. The CH505 TF envelope did not bind to the CH235 precursor antibody, which is called the CH235 unmutated common ancestor (UCA). In neutralization screens of different viruses, it was discovered that N279K substitution in the CH505 TF virus improved CH235 UCA neutralization (LeBranche et al. 2019 Plos Pathogens, Sep. 17, 2019, https://doi.org/10.1371/journal.ppat.1008026). Throughout this patent application CH505 TF N279K, which is CH505 TF envelope sequence comprising N279K substitution is interchangeably referred also as CH505 M5 and/or CH505 TF M5(N279K). Further screening of viruses showed that CH235 UCA neutralized CH505 TF N279K plus G458Y mutant viruses more potently than N279K alone (LeBranche et al. supra; see also FIG. 1). As shown for other antibodies against the same epitope, enriching for mannose glycans on HIV-1 envelope further improved CH235 neutralization of the CH505 TF N279K G458Y virus (LeBranche et al supra). Recombinant HIV-1 envelopes were made that recapitulated the results seen with virus neutralization (LeBranche et al supra). Mannose enrichment is achieved by production of virus or proteins in GnT1− cell lines. See Eggink et al. Virology. 2010 Jun. 5; 401(2): 236-247. In contrast, HIV-1 envelope produced in cells with a natural glycan biosynthesis pathway have a mixture of processed and mannose glycans at different glycosylation sites (Pritchard L K et al. Cell Rep. 2015 Jun. 16; 11(10):1604-13. doi: 10.1016/j.celrep.2015.05.017. Epub 2015 Jun. 4).

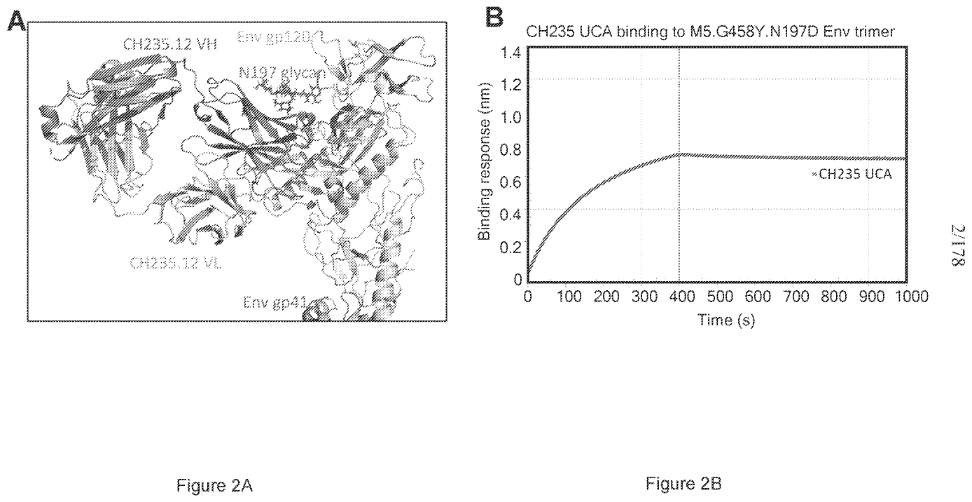

While mannose enrichment is possible for purified recombinant protein, it is not possible to enrich for specific glycosylation on protein produced in vivo after nucleic acid immunization. To understand why mannose enrichment improved CH235 binding to envelope we inspected the structure of CH235 UCA bound to envelope for glycans proximal to its epitope—the CD4 binding site. The glycan attached at position N197 reached across the heavy chain framework 1 region of CH235 (FIG. 2A). We hypothesized that this glycan impeded CH235 binding and that shortening the glycan to a trimmed mannose glycan would allow better binding to the CH235 lineage antibodies. We hypothesized that if the N197 glycan is “removed”, then mannose enrichment of envelope would not be required and thus permitting delivery of the envelope by nucleic acid immunization.

Removal of the glycan is achieved by introducing a non-N-glycosylatable amino acid instead of the asparagine at position 197. A N197D substitution was introduced into the CH505 TF N279K G458Y virus. CH235 UCA neutralization of heterogeneously glycosylated (ie not produced in GNT1− cell lines but rather produced in 293F cells) CH505 TF N279K/G458Y/N197D virus was compared to mannose-enriched CH505 TF N279K/G458Y. The CH235 UCA neutralized both viruses potently with the concentration of antibody required to neutralize 50% of virus replication differing by only 3 fold between the two viruses (FIG. 1). Thus, removal of the N197 glycan with a N197D substitution or mannose enrichment conferred comparable sensitivity of the CH505 TF N279K/G458Y to CH235 UCA neutralization. The CH235 UCA bound to heterogeneously glycosylated CH505 TF N279K/G458Y/N197D recombinant envelope proteins (FIG. 2B). Overall, N197D could therefore be introduced into envelope instead of enriching for mannose glycosylation.

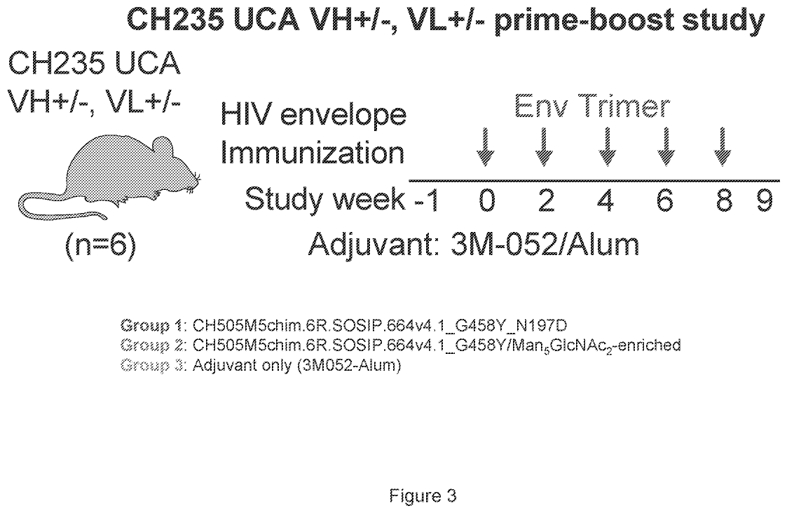

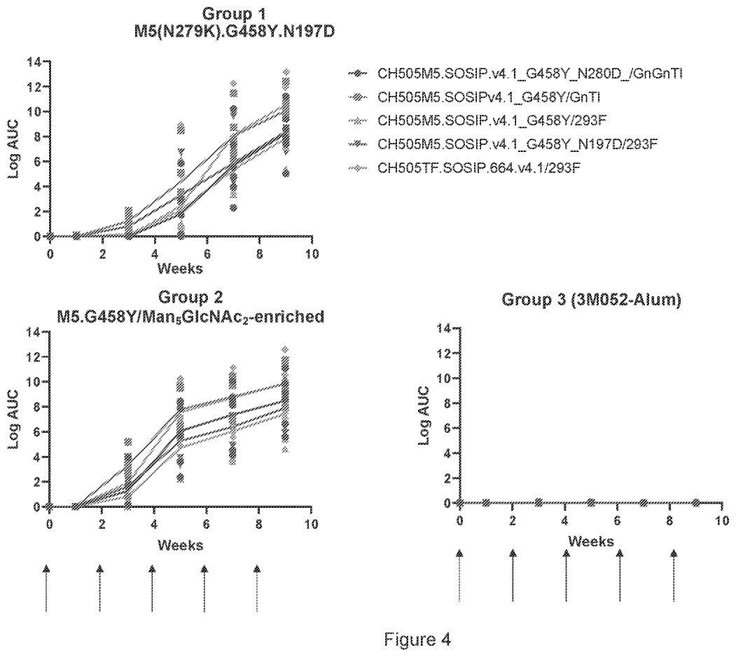

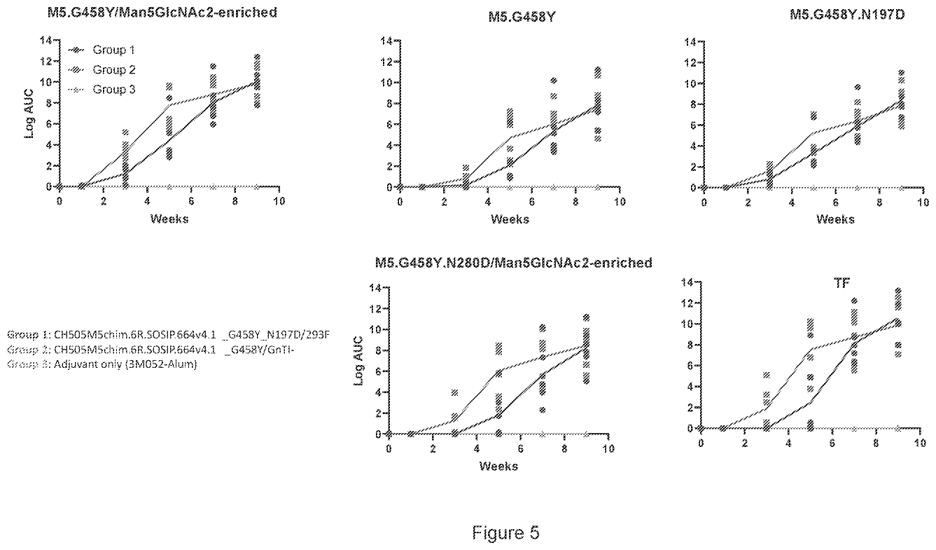

We compared immunogenicity of heterogeneously glycosylated CH505 TF N279K/G458Y/N197D (produced in 293 cells) and mannose-enriched CH505 TF N279K/G458Y (produced in GnT1−) recombinant gp140 envelopes in mice expressing the CH235 UCA B cell receptor (FIG. 3). One group of mice received the heterogeneously glycosylated CH505 TF N279K/G458Y/N197D recombinant gp140 envelope (HV1301288_G458Y_N197D), and a second group of mice received the mannose-enriched CH505 TF N279K/G458Y recombinant gp140 envelope (HV1301288_G458Y). All mice received five 25 microgram doses of recombinant protein with GLA-SE as the adjuvant. Binding antibodies to CH505 gp140 trimer proteins were elicited, which rose with subsequent immunizations (FIGS. 4,5).

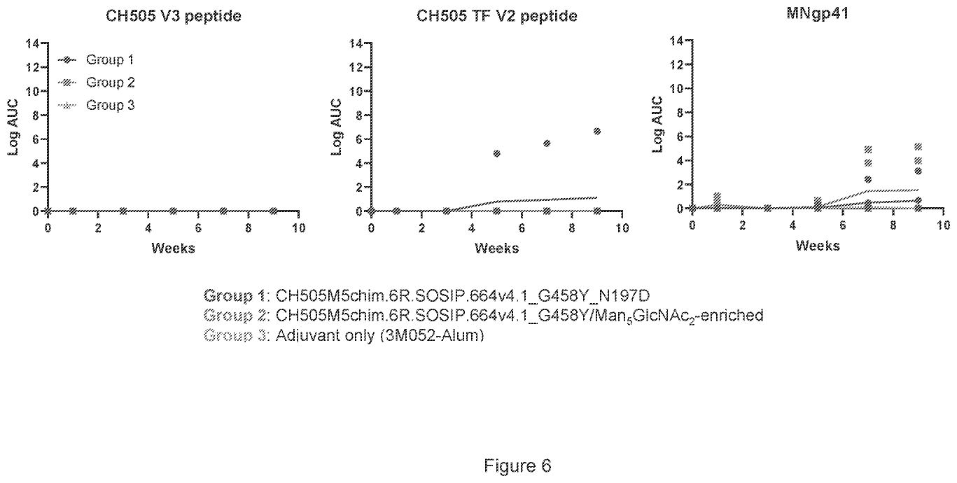

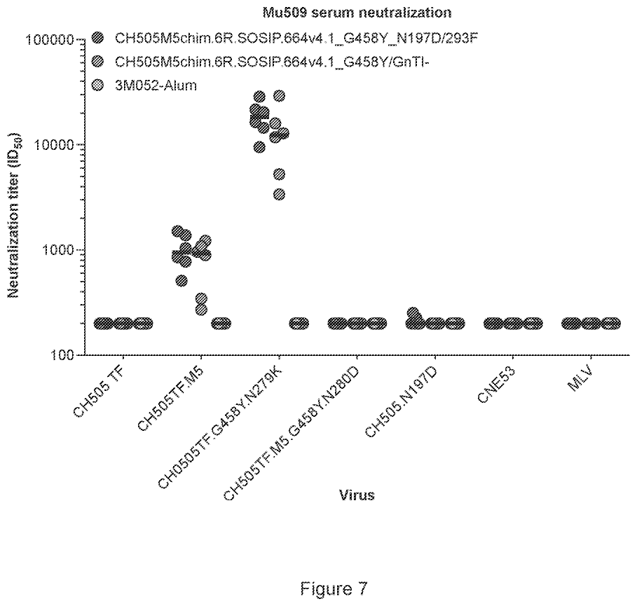

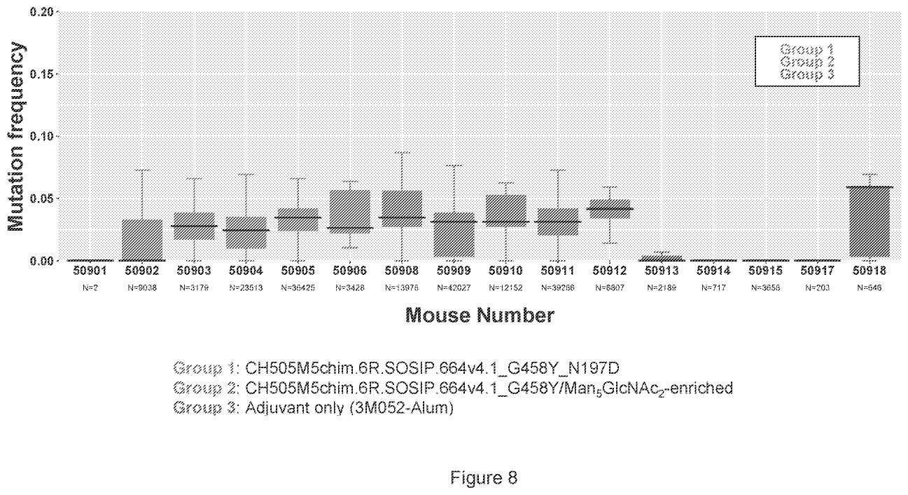

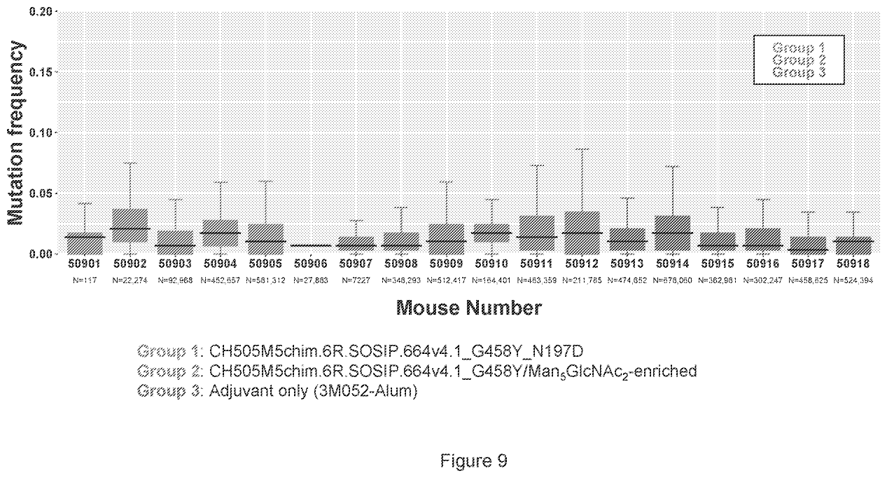

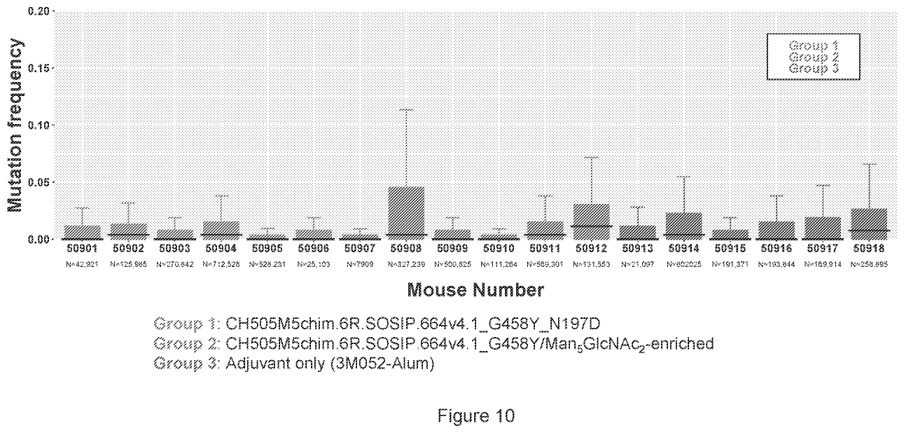

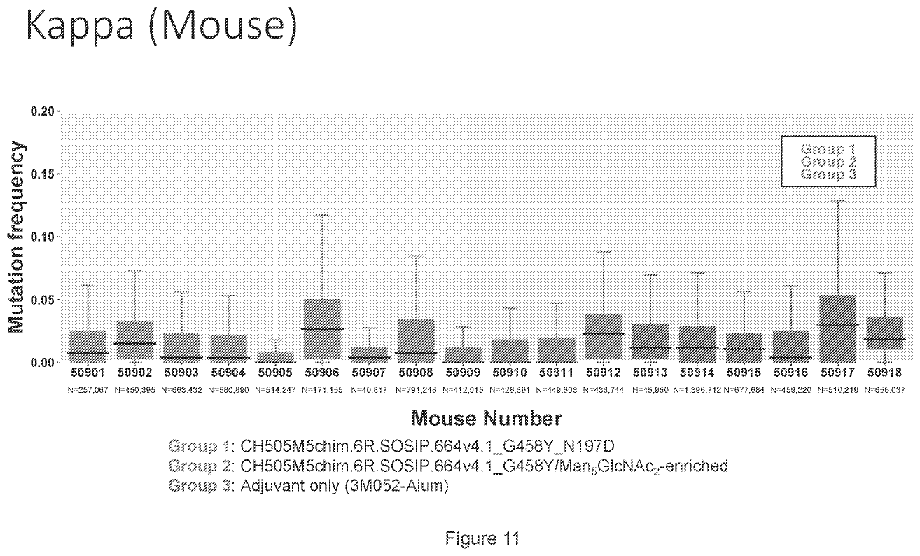

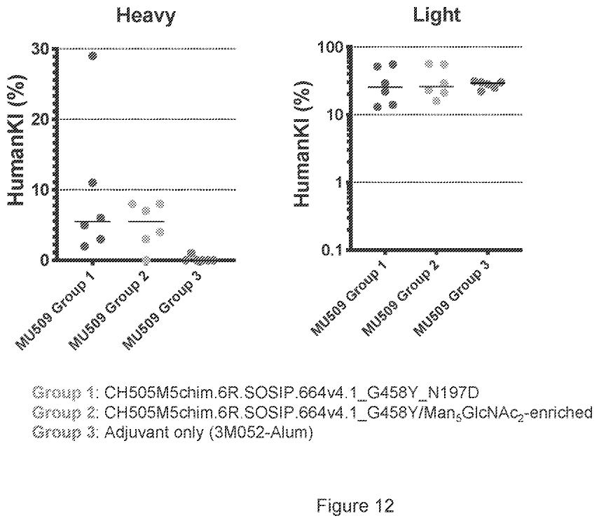

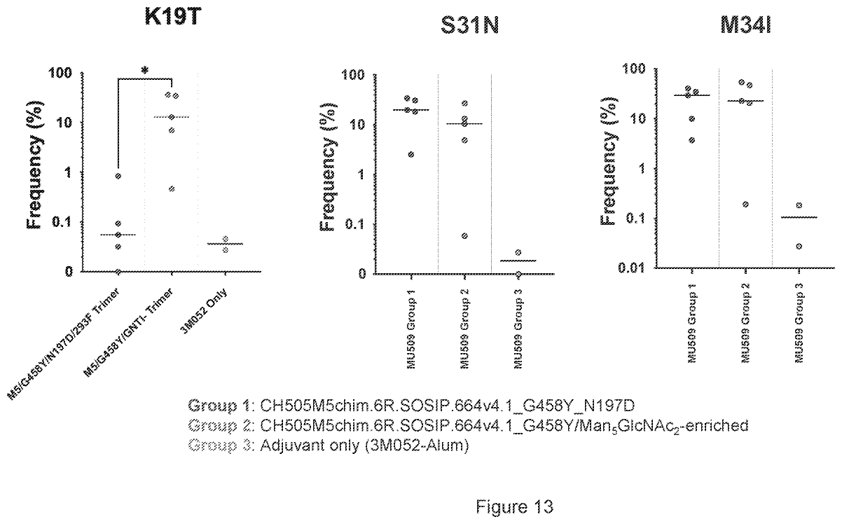

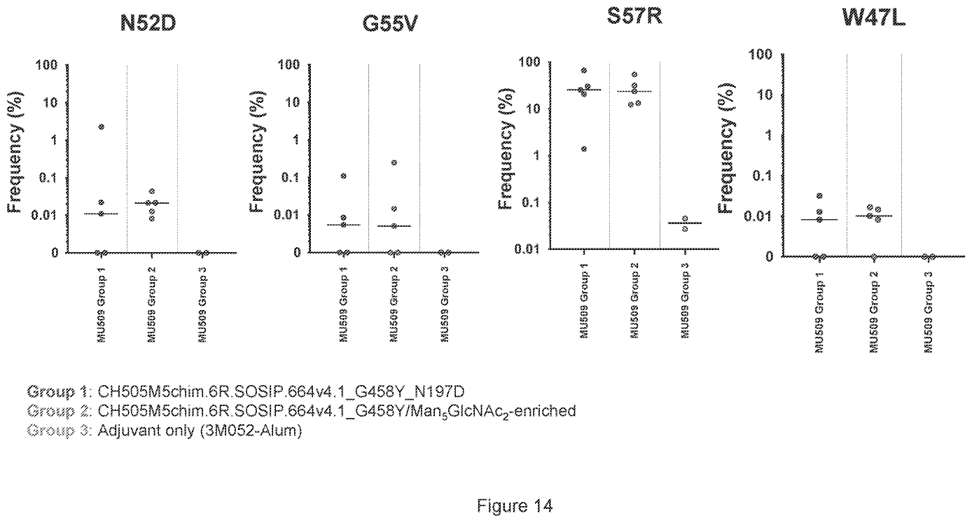

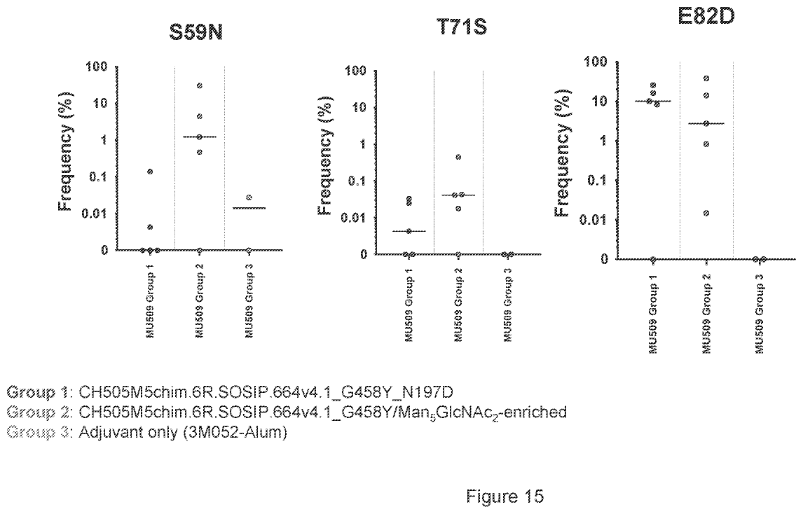

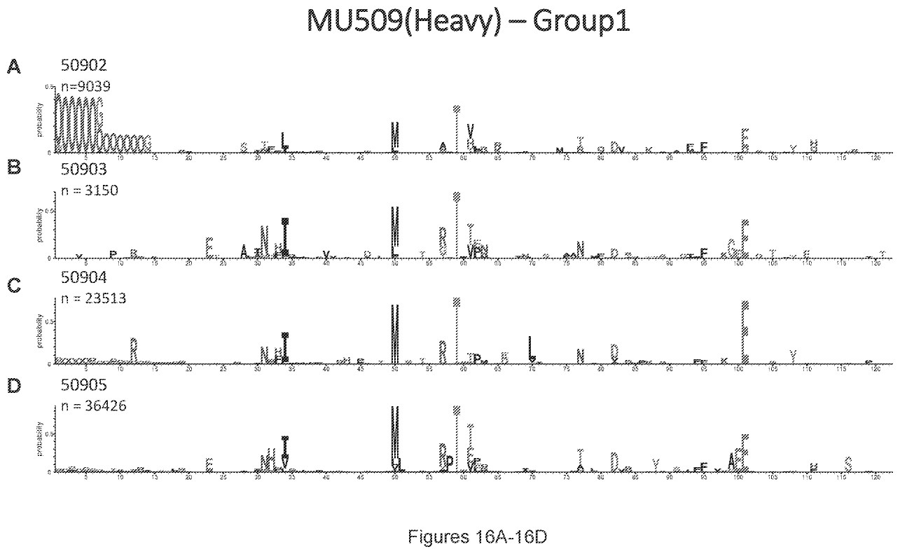

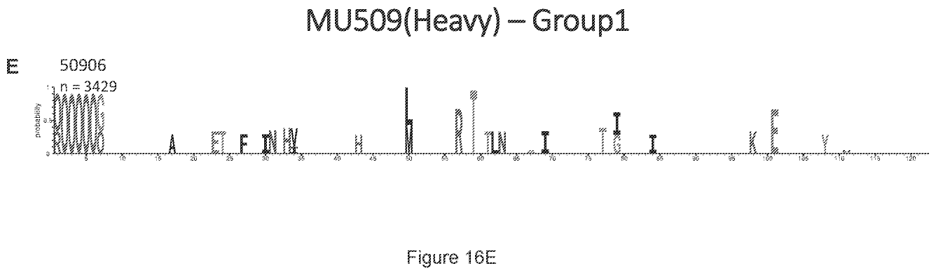

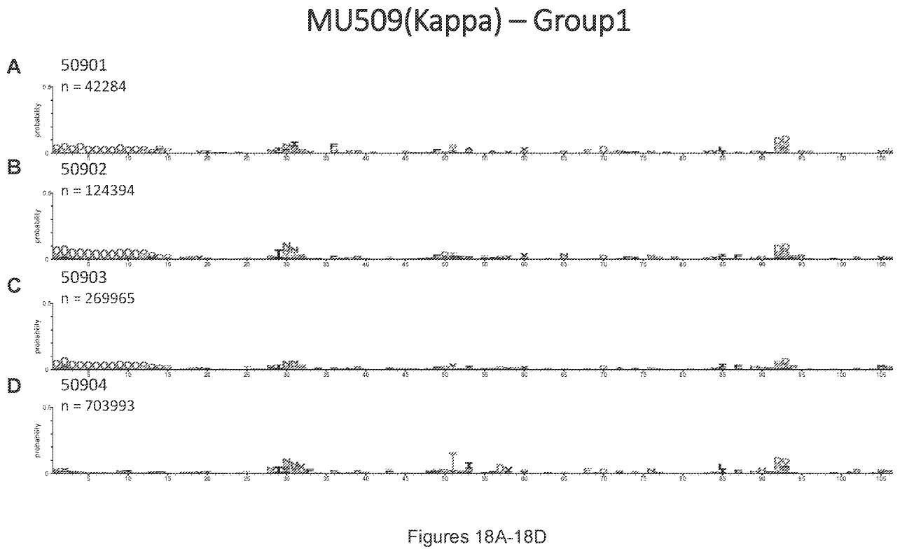

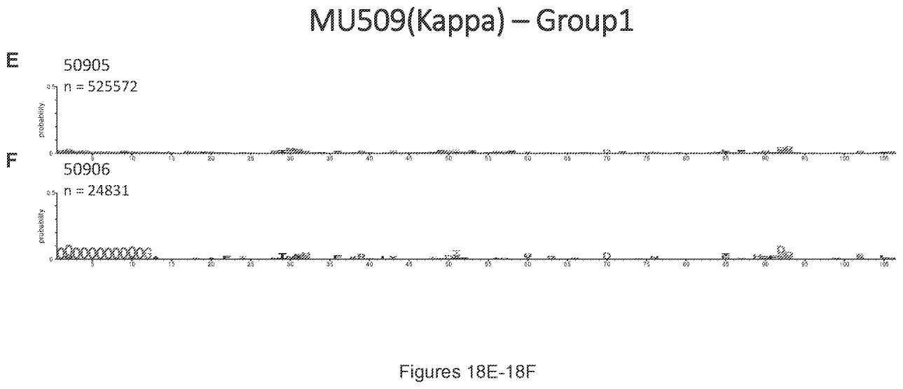

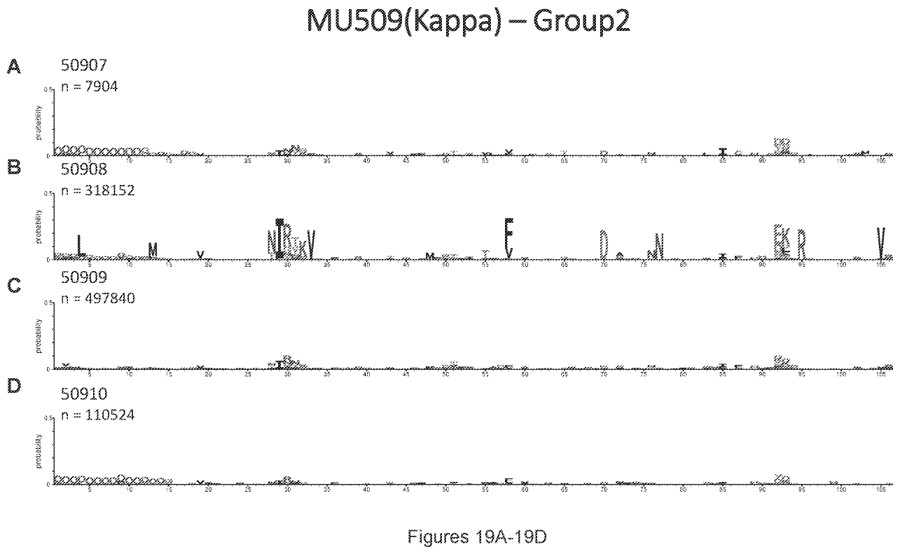

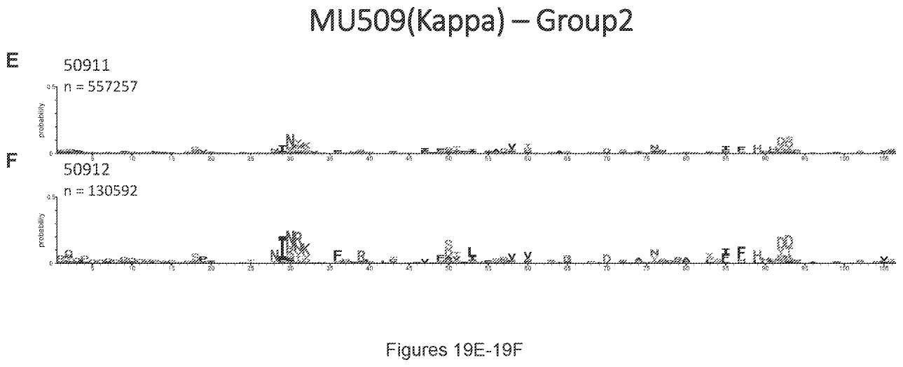

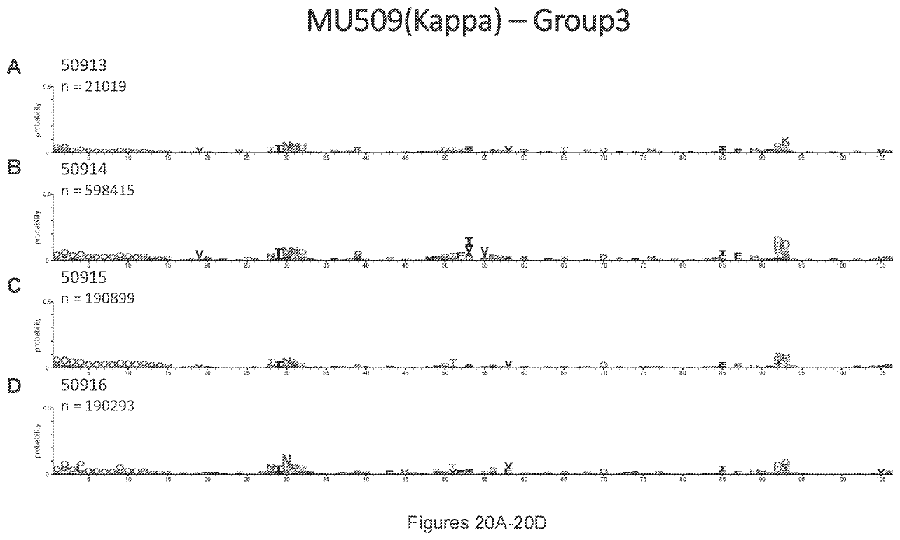

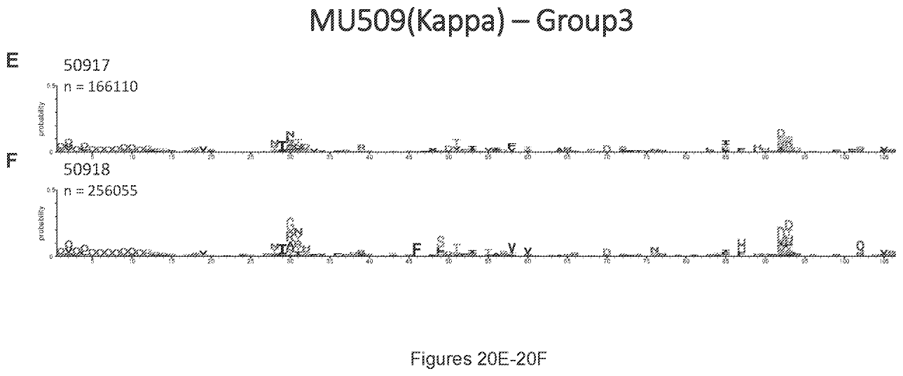

Only low titers of antibodies were elicited against linear V3, linear V2 or gp41 peptides (FIG. 6). After 5 immunizations neutralization titers against CH505 TF N279K/G458Y and CH505 TF M5 viruses were comparable for both groups of immunized mice (FIG. 7). Neutralization was sensitive to N280D substitution indicating it was CD4 binding site directed (FIG. 7). Next generation sequencing of CH235 antibodies determined vaccination generated mutated CH235 VH gene segments, but mouse VH and VK gene segments showed little somatic mutation (FIGS. 8-11). Both groups of envelope immunized mice had similar percentages of CH235 antibodies (FIG. 12). The somatic mutation showed that the vaccination initiated the affinity maturation process. Individual mutations were analyzed and key mutations that occur in the CH235 lineage were also observed among the sequences from the envelope-immunized mice (FIGS. 13-20). The frequency of amino acid substitution due to somatic mutation was similar for both groups of vaccinated mice except for the K19T frequency. The substitution of threonine at position 19 allows for accommodation of the N197 glycan. Since the N197D eliminated the glycan, selective pressure for the K19T mutation would be expected to decrease. Consistent with this hypothesis, the group of mice administered the N197D envelope had a lower frequency of CH235 sequences with the K19T substitution (FIG. 13). In summary, immunization with either envelope generated similar antibody neutralization and somatic mutation in the CH235 UCA knock-in mouse model.

Optimized HIV-1 CH505 TF N279K/G458Y/N197D envelopes were designed as vaccine immunogens to be delivered by nucleic acid or viral vectors. The envelopes were designed as gp160s (untruncated), soluble gp140s (truncated at position 664), and gp140 H. pylori ferritin nanoparticles (FIG. 21 and FIG. 22).

In some embodiments, the envelope was stabilized in the prefusion conformation by either introducing H66A A582T L587A substitutions and/or by introducing A204V V208 V68I V255L (F14) substitutions. In some embodiments, to increase gp160 presentation on the cell surface a Y712I substitution was introduced into all gp160 sequences since Y712 mediated clathrin-dependent endocytosis. In some embodiments, gp160 sequences were further stabilized by substituting cysteines at amino acids 501 and 605 to form a disulfide bond between gp120 and gp41. In some embodiments, to keep gp120 associated with gp41, the furin cleavage site was replaced with a flexible linker (GGGGSGGGGS). In some embodiments, the unstable loop between the central helix and heptad repeat 1 was replaced with a flexible 23 amino acid loop (GSAGSAGSGSAGSGSAGSGSAGS).

In some embodiments, to further improve interprotomer interactions in a subset of envelope designs, all interprotomer interacting amino acids were identified. All interprotomer contacts matched the highly stable envelope BG505 except amino acid 1535. A I535M substitution was introduced to further stabilize interprotomer interactions. Soluble gp140 envelopes were made as chimeric SOSIP envelopes stabilized with F14 mutations to not undergo conformational changes upon CD4 binding (See WO/2020/072169 for further specific information regarding F14 mutations).

An additional set of trimers were made that were not chimeras and were derived from CH505 envelope sequence. The non-chimeric envelopes were stabilized with the new disulfide bond, flexible linker between gp120 and gp41, flexible linker between the central helix and HR1, F14 mutations, and I535M substitutions as described for the gp160 constructs. The bottom of the trimer is unshielded by glycans and is not conformationally masked resulting in dominant recognition by non-neutralizing antibodies (Crotty et al.). New glycosylation sites at positions 630, 657, and 665 (3Gly) or 657 and 665 (2Gly) were added to the trimer to cover the base of the trimer with glycans thereby reducing its exposure (Kulp et al. Structure-based design of native-like HIV-1 envelope trimers to silence non-neutralizing epitopes and eliminate CD4 binding. Kulp D W, Steichen J M, Pauthner M, Hu X, Schiffner T, Liguori A, Cottrell C A, Havenar-Daughton C, Ozorowski G, Georgeson E, Kalyuzhniy O, Willis J R, Kubitz M, Adachi Y, Reiss S M, Shin M, de Val N, Ward A B, Crotty S, Burton D R, Schief W R. Nat Commun. 2017 Nov. 21; 8(1):1655. doi: 10.1038/s41467-017-01549-6.PMID: 29162799). Both chimeric SOSIP gp140 and nonchimeric gp140s were arrayed on the surface of ferritin nanoparticles by fusing the envelope gene to the gene for aglycosylated H. pylori ferritin. The ferritin lacked the first 4 amino acids at the N-terminus and had an additional GS linker on the C-terminus. The envelope was fused to the ferritin gene using a 10, 14, or 25 amino acid linker. The linkers were a combination of flexible loops and more rigid alpha helices. A set of CH505 TF envelopes without the N279K, G458Y, and N197D substitutions were also designed for use as immunogens.

In non-limiting embodiments, any of these envelopes could be immunogens delivered as recombinant proteins, and/or by nucleic acid, including without limitation modified mRNAs, and/or viral vectors, including without limitation self-replicating viral vectors.

### Example 3 Animal Studies

In non-limiting embodiment these immunogens can be used as either single primes and boosts in humanized mice or bnAb UCA or intermediate antibody VH+VL knockin mice, non-human primates (NHPs) or humans, or used in combinations in animal models or in humans.

In non-limiting embodiments, these are administered as recombinant protein. Any suitable adjuvant could be use. In non-limiting embodiments, these are administered as nucleic acids, DNA and/or mRNAs. In non-limiting embodiments, the mRNAs are modified mRNAs administered as LNPs.

In non-limiting embodiments, the immunogens provide optimal prime for CD4 binding site precursors. In some embodiments, an optimal prime is determined by measurement of the frequency of bnAb precursors before immunization and after each immunization to determine if the immunization has expanded the desired bnAb B cell precursor pool. This can be performed by initial B cell repertoire analysis by single cell sorting of memory or germinal center B cells (e.g. Bonsignori et al. Sci Transl Med. 2017 Mar. 15; 9(381): eaai7514.) and then followed by next generation sequencing of either lymph node, blood or other immune organ B cells to determine if the primed B cell bnAb clones were expanded and therefore boosted.

1. A recombinant HIV-1 envelope polypeptide selected from the group consisting of HV1302791, HV1302934, HV1302920, HV1301288_G458Y_N197D, and HV1303443, wherein the polypeptide is a non-naturally occurring protomer designed to form an envelope trimer.

2. The recombinant HIV-1 envelope polypeptide of claim 1, wherein the envelope is a gp160 transmembrane envelope.

3. The recombinant HIV-1 envelope polypeptide of claim 1, wherein the envelope is comprised in a multimeric complex.

4. The recombinant HIV-1 envelope polypeptide of claim 1, wherein the envelope is based on CH505 T/F envelope and comprises an optimized sequence for binding to CH235 UCA.

5. The recombinant HIV-1 envelope polypeptide of claim 1, wherein the envelope comprises mutations G458Y and N197X, wherein X is any amino acid which does not allow glycosylation.

6. The recombinant HIV-1 envelope polypeptide of claim 1, wherein the envelope comprises mutations G458Y and N197D.

7. The recombinant HIV-1 envelope polypeptide of claim 1, wherein the envelope further comprises F14 mutations A204V, V208L, V681, and V255L.

8. The recombinant HIV-1 envelope polypeptide of claim 1, wherein the envelope further comprises any changes at positions of interprotomer contacts within a single trimeric envelope.

9. The recombinant HIV-1 envelope polypeptide of claim 8, wherein the envelope comprises 1535M.

10. The recombinant HIV-1 envelope polypeptide of claim 1, wherein the envelope is linked via a linker to a self-assembling protein to form a fusion protein.

11-13. (canceled)

14. A nucleic acid encoding the recombinant HIV-1 envelope polypeptide of claim 1.

15. A recombinant trimer comprising three identical protomers of the envelope of claim 1.

16. An immunogenic composition comprising the recombinant trimer of claim 15 and a carrier.

17. An immunogenic composition comprising a nucleic acid encoding the recombinant HIV-1 envelope of claim 1 and a carrier.

18. The immunogenic composition of claim 17, wherein the recombinant HIV-1 envelope is in the form of a trimer, and/or nanoparticle.

19. The immunogenic composition of claim 16 further comprising an adjuvant.

20. The immunogenic composition of claim 17 wherein the nucleic acid is inserted in an expression vector.

21. A method of inducing an immune response in a subject comprising administering in an amount sufficient to affect such induction the immunogenic composition of claim 16 in an amount sufficient to induce an immune response.

22-27. (canceled)

28. A composition comprising a nanoparticle and a carrier, wherein the nanoparticle comprises the recombinant HIV-1 envelopes of claim 1.

29. (canceled)

30. A composition comprising a nanoparticle and a carrier, wherein the nanoparticle comprises the trimer of claim 15.

31-42. (canceled)
